(19) United States
(12) Patent Application Publication
(10) Pub. No.: US 2008/0200376 A1
(43) Pub. Date: Aug. 21, 2008

(54) Compositions and Methods For the Treatment Of Obesity and Sexual Dysfunction

(76) Inventors: Malcolm MacCoss, Freehold, NJ (US); Duncan E. MacIntyre, Scotch Plains, NJ (US); Alison M. Strack, Bloomfield, NJ (US)

Correspondence Address: MERCK AND CO., INC, P O BOX 2000, RAHWAY, NJ, 07065-0907, US

(21) Appl. No.: 11/665,127

(22) PCT Filed: Oct. 25, 2005
(86) PCT No.: PCT/US2005/038194

§ 371 Date: Oct. 21, 2007

(30) Foreign Application Priority Data
Oct. 29, 2004 (US) 60623405

(51) Int. Cl. A61K 38/22; A61K 31/155; A61K 31/485; A61K 31/357; A61K 31/42; A61K 31/473; A61K 31/519; A61P 3/00; A61P 35/00; A61P 9/00; A61P 15/00

(52) U.S. Cl. 514/12; 514/635; 514/282; 514/455; 514/397; 514/284; 514/262.1

(57) ABSTRACT

The present invention relates to methods of treating and preventing obesity and obesity-related disorders in a subject comprising administering a neurokinin-1 antagonist and an anti-obesity agent, such as a melanocortin 4 receptor agonist, to said subject. The present invention further related to methods of treating or preventing sexual dysfunction, including male erectile dysfunction, in a subject comprising administering a neurokinin-1 antagonist and a sexual dysfunction therapeutic agent, such as a melanocortin 4 receptor agonist, to said subject. The present invention further provides for pharmaceutical compositions and medicaments useful in carrying out these methods.

### BACKGROUND OF THE INVENTION

It is estimated that about 97 million adults in the United States are overweight or obese. The medical problems associated with obesity, which can be serious and life-threatening, include hypertension; type 2 diabetes mellitus; elevated plasma insulin concentrations; insulin resistance; dyslipidemias; hyperlipidemia; endometrial, breast, prostate and colon cancer; osteoarthritis; respiratory complications, such as obstructive sleep apnea; cholelithiasis; gallstones; arteriosclerosis; heart disease; and heart arrythmias (Kopelman, P. G., Nature 404, 635-643 (2000)). Obesity is further associated with premature death and with a significant increase in mortality and morbidity from stroke, myocardial infarction, congestive heart failure, coronary heart disease, and sudden death. Obesity also exacerbates many health problems, both independently and in association with other diseases. Obesity is a major health concern in Western societies.

The melanocortin 4 receptor is implicated in the control of food intake and energy expenditure, and in modulating erectile function and sexual behavior (Van der Ploeg et al., PNAS, Vol. 99, No. 17, 11381-11386 (2002); Martin et al., European Urology, Vol. 45, Issue 6, 706-713 (2004). Pro-opiomelanocortin (POMC) derived peptides are known to affect food intake. Five distinct MC-R's have thus far been identified (MC1R, MC2R, MC3R, MC4R and MC5R), and these are expressed in different tissues. MC4R is uniquely expressed in the brain, and its inactivation was shown to cause obesity (Huszar et al., Targeted disruption of the melanocortin-4 receptor results in obesity in mice; Cell 88:131-141 (1997); A. Kask, et al., “Selective antagonist for the melanocortin-4 receptor (HS014) increases food intake in free-feeding rats,” Biochem. Biophys. Res. Commun., 245: 90-93 (1998)). A specific single MC-R that may be targeted for the control of obesity has not yet been identified, although evidence has been presented that MC-4R signalling is important in mediating feed behavior (S. Q. Giraudo et al., “Feeding effects of hypothalamic injection of melanocortin-4 receptor ligands,” Brain Research, 80: 302-306 (1998)).

Weight loss drugs that are currently used to treat obesity have limited efficacy. Studies of the weight loss medications orlistat (Davidson, M. H. et al. (1999) JAMA 281:235-42), dexfenfluramine (Guy Grand, B. et al. (1989) Lancet 2:1142-5), sibutramine (Bray, G. A. et al. (1999) Obes. Res. &:189-98) and phentermine (Douglas, A. et al. (1983) Int. J. Obes. 7:591-5) have demonstrated a limited weight loss of about 5%-10% of body weight for drug compared to placebo. The side effects of these anti-obesity agents further limit their use. Dexfenfluramine was withdrawn from the market because of suspected heart valvulopathy; orlistat is limited by gastrointestinal side effects; the use of topiramate is limited by central nervous system effects; and the use of sibutramine is limited by its cardiovascular side effects which have led to reports of deaths and its withdrawal from the market in Italy.

Melanocortin receptor involvement in male and female sexual dysfunction has also been reported. Approximately 140 million men worldwide suffer from impotency or erectile dysfunction. Erectile dysfunction or “impotence” denotes the medical condition of inability to achieve penile erection sufficient for successful sexual intercourse. Erectile dysfunction can arise from either organic or psychogenic causes, with about 20% of such cases being purely psychogenic in origin. Erectile dysfunction increases from 40% at age 40, to 67% at age 75, with over 75% occurring in men over the age of 50.

Synthetic melanocortin receptor agonists (melanotropic peptides) have been found to initiate erections in men with psychogenic erectile dysfunction [See H. Wessells et al., “Synthetic Melanotropic Peptide Initiates Erections in Men with Psychogenic Erectile Dysfunction: Double-Blind, Placebo Controlled Crossover Study,” J. Urol., 160: 389-393 (1998); Fifteenth American Peptide Symposium, Jun. 14-19, 1997 (Nashville Tenn.)]. Activation of melanocortin receptors of the brain appears to cause normal stimulation of sexual arousal. In the above study, the centrally acting α-melanocyte-stimulating hormone analog, melanotan-II (MT-II), exhibited a 75% response rate when injected intramuscularly or subcutaneously to males with psychogenic erectile dysfunction. MT-II (PT-14; Erectide®) is a synthetic cyclic heptapeptide, Ac-Nle-c[Asp-His-DPhe-Arg-Trp-Lys]-NH₂, which is a non-selective MC-1R, -3R, 4R, and -5R agonist (Dorr et al., Life Sciences, Vol. 58, 1777-1784, 1996). Adverse reactions observed with MT-II include nausea, flushing, loss of appetite, stretching, and yawning and may be the result of activation of MC-1R, MC-2R, MC-3R, and/or MC-5R. MT-II must be administered parenterally, such as by subcutaneous, intravenous, or intramuscular route, since it is not absorbed into the systemic circulation when given by the oral route.

Compositions of melanotropic peptides and methods for the treatment of psychogenic erectile dysfunction are disclosed in U.S. Pat. No. 5,576,290, assigned to Competitive Technologies. Methods of stimulating sexual response in females using melanotropic peptides have been disclosed in U.S. Pat. No. 6,051,555.

A major concern with the use of anti-obesity agents and sexual dysfunction therapeutic agents is the side effect of emesis. Recent studies have shown that non-selective melanocortin 4 receptor agonists, such as MT-II and PT-141, induce nausea and emesis (Life Sciences, 58: 1777-1784, 1996; Intl. J. Impotence Res. 16, 51-59, 2004). In the PT-141 trial, serotonin-3 receptor antagonist, ondansetron, was shown to reduce nausea experienced by patients undergoing PT-141 therapy. Human pharmacokinetic trials have also shown this nausea and emesis side effect induced by selective melanocortin 4 receptor agonists when given for the treatment obesity and sexual dysfunction. These emesis side effects may lead to lower patient compliance during the treatment of obesity and sexual dysfunction with anti-obesity agents and sexual dysfunction therapeutic agents, such as melanocortin agonists, known to cause emesis.

Substance P is a naturally occurring undecapeptide belonging to the tachykinin family of peptides, the latter being so-named because of their prompt contractile action on extravascular smooth muscle tissue. The tachykinins are distinguished by a conserved carboxyl-terminal sequence. In addition to substance P, the known mammalian tachykinins include neurokinin A and neurokinin B. The current nomenclature designates the receptors for substance P, neurokinin A, and neurokinin B as neurokinin-1 (NK-1), neurokinin-2 (NK-2), and neurokinin-3 (NK-3) respectively. The biological actions of these tachykinins are mediated through specific cell-surface receptors, designated NK₁, NK₂ and NK₃. These have been confirmed by the cloning of three distinct genes from various mammalian sources, including man, with SP being the preferred agonist for NK₁ receptors, neurokinin A the preferred agonist at NK₂ and neurokinin B the preferred agonist at NK₃ although each of these tachykinins can act as agonists at all three receptors. Neurokinin-1 receptor antagonists are being developed for the treatment of numerous physiological disorders associated with an excess or imbalance of tachykinins, and in particular substance P, including disorders of the central nervous system such as anxiety, depression and psychosis as disclosed in WO 95/16679, WO 95,18124, and WO 95/23798). International Patent Application No. WO 96/24353 discloses a combination of a tachykinin antagonist and a serotonin agonist or selective serotonin reuptake inhibitor (SSRI) to treate psychiatric disorders. NK-1 antagonists are also efficacious in blocking emesis induced by a range of stimuli in a range of preclinical species, including ferrets and have been shown to inhibit post operative and cancer chemotherapy-induced emesis in humans (PLR Andrews and J A Rudd 2004 Handbook of Experimental Pharmacology (Ed Holzer P.) The role of Tachykinins and the Tachykinin NK1 receptor in nausea and vomiting pp 359-440).

International Application No. WO 98/47513 and U.S. Pat. No. 6,162,805 disclose the use of an NK-1 receptor antagonist, optionally with an anoretic agent, for the manufacture of a medicament for the treatment and prevention of eating disorders, such as obesity, bulima, and compulsive eating disorder. This patent specifically discloses the use of a NK-1 receptor antagonist with an anti-obesity agent selected from amfepramone (diethylpropion), a minorex; amphechloral; amphetamine; benzphetamine; chlorphenternine; clobenzorex; cloforex; clominorex; clortermine; cyclexedrine; dexfenfluramine; dextroamphetamine; diphemethoxidine, N-ethylamphetamine; fenbutrazate; fenfluramine; fenisorex; fenproporex; fludorex; fluminorex; furfurylmethylamphetamine; levamfetamine; levophacetoperane; mazindol; mefenorex; metamfepramone; methamphetamine; norpseudoephedrine; pentorex; phendimetrazine; phenmetrazine; phentermine; phenylpropanolamine; picilorex, and sibutramine for the manufacture of a medicament for the treatment and prevention of eating disorders. However, the treatment of obesity and obesity related disorders with a composition comprising a neurokinin-1 antagonist and a melanocortin 4 receptor antagonist is not disclosed.

U.S. Pat. No. 5,929,054 discloses the use of neurokinin-1 receptor antagonists to treat or prevent sexual dysfunction. However, the treatment of sexual dysfunction with a composition comprising a neurokinin-1 antagonist and a melanocortin 4 receptor agonist is not disclosed. There is no disclosure of a combination of a neurokinin-1 antagonist and a selective melanocortin 4 receptor agonist to treat obesity, obesity related disorders or sexual dysfunction.

Despite recent advances, there is a continuing need for new and improved methods of treating and preventing obesity and obesity-related disorders. Due to the limited efficacy of currently available for mono- and combination therapies there is also a need for a combination obesity treatment with enhanced efficacy, and fewer undesirable side effects, such as nausea and emesis. The instant invention addresses this problem by providing a combination therapy comprised of an anti-obesity agent, such as a melanocortin-4 agonist, and a neurokinin-1 antagonist useful in the treatment and prevention of obesity and obesity-related disorders.

There is also a continuing need in the medical arts for improved methods and compositions to treat individuals suffering from psychogenic and/or organic sexual dysfunction with enhanced convenience and ease of compliance, while minimizing or inhibiting the nausea and emesis side effects of the sexual dysfunction therapeutic agents currently available. The instant invention addresses this problem by providing a combination therapy comprised of a sexual dysfunction therapeutic agent, such as a melanocortin 4 receptor agonist, and a neurokinin-1 receptor antagonist useful in the treatment and prevention of sexual dysfunction, including male erectile dysfunction and female sexual dysfunction, with fewer undesirable side effects, such as emesis.

It has now been found that a combination of a neurokinin-1 antagonist and an anti-obesity agent is useful to treat and prevent obesity and obesity-related disorders. In particular it has been surprisingly found that a combination of a neurokinin-1 receptor antagonist and a melanocortin 4 agonist is useful to treat and prevent obesity and obesity-related disorders, while reducing the emesis side effect caused by administration of the anti-obesity agent. Such combinations exhibit unexpected and advantageous results, for example, providing additional reduction in food intake and bodyweight while minimizing the side effects of nausea and/or emesis associated with melanocortin 4 agonist therapy. It has also been surprisingly found that the combination of a neurokinin-1 antagonist and an anti-obesity agent, such as a melanocortin-4 receptor agonist, is advantageous in the treatment of obesity over treatment with either a neurokinin-1 antagonist or the anti-obesity agent alone.

Treatment with the compositions of the present invention allows the use of a subclinical dose of a neurokinin-1 antagonist and a sub-clinical dose of an anti-obesity agent or sexual dysfunction therapeutic resulting in effective treatment with fewer side effects, such as emesis, than current monotherapies. Additionally, treatment with the compositions of the present invention allow the use of larger doses of the anti-obesity agent or sexual dysfunction therapeutic, such as a melanocortin 4 agonist, in cases where high doses are limited due to dose related nausea and/or emesis side effects.

The use of selective melanocortin 4 receptor agonists is beneficial over the use of non-selective melanocortin agonists since selective MC4R agonists do not exhibit the side effects associated with non-selective MC4R agonists, such as melanocortin 1 mediated pigmentation changes and worsening of acne associated with MC5R agonists. Additionally, the use of non-peptidyl melanocortin-4 receptor agonists for the treatment and prevention of obesity and obesity-related disorders is beneficial over the use of peptidyl melanocortin 4 agonists, which exhibit decreased oral bioavailability due to their degradation in the stomach and GI tract, and which exhibit decreased brain penetration relative to non-peptidyl melanocortin 4 agonists. However, both peptidyl and non-peptidyl melanocortin 4 agonists may be used in the methods of the present invention.

It is an object of the present invention to identify methods of treating and preventing obesity and obesity-related disorders comprising administration of a neurokinin-1 antagonist and an anti-obesity agent, such as a melanocortin 4 receptor agonist, to a subject. It is another object of the present invention to identify methods of treating and preventing obesity and obesity-related disorders while reducing the occurrence of emesis comprising administration of a neurokinin-1 antagonist and an anti-obesity agent, such as a melanocortin 4 receptor agonist, to a subject. It is another object of the present invention to identify methods of reducing the emesis side effect during the treatment and prevention of sexual dysfunction comprising administration of a neurokinin-1 antagonist and a sexual dysfunction therapeutic, such as a melanocortin 4 receptor agonist, to a subject. It is a further object of the present invention to provide a method of manufacture of a medicament useful to treat and prevent obesity, obesity-related disorders, and sexual dysfunction.

### SUMMARY OF THE INVENTION

The present invention provides compositions comprising a neurokinin-1 antagonist and an anti-obesity agent useful in the treatment or prevention of obesity, and obesity-related disorders. The present invention further provides compositions comprising a neurokinin-1 antagonist and a sexual dysfunction therapeutic useful in the treatment or prevention of sexual dysfunction, including male erectile dysfunction and female sexual dysfunction.

The present invention is further concerned with compositions comprising a neurokinin-1 receptor antagonist, and an anti-obesity agent selected from the group consisting of: 5HT transporter inhibitor; norepinephrine (NE) transporter inhibitor; CB-1 antagonist/inverse agonist; ghrelin antagonist; H3 antagonist/inverse agonist; MCHIR antagonist; MCH2R agonist/antagonist; MC3R agonist; MC4R agonist; neuromedin U 1 receptor agonist; neuromedin U 2 receptor agonist; NPY1 antagonist; NPY2 agonist; NPY4 agonist; NPY5 antagonist; leptin; leptin agonist/modulator; leptin derivatives; opioid antagonist; orexin antagonist; BRS3 agonist; 11β HSD-1 inhibitor, CCK-A agonist; CNTF; CNTF agonist/modulator; CNTF derivative; Cox-2 inhibitor; DP-IV inhibitor; GHS agonist; 5HT2C agonist; 5HT6 antagonist; monoamine reuptake inhibitor; UCP-1, 2, and 3 activator; 03 agonist; thyroid hormone β agonist; FAS inhibitor; DGAT1 inhibitor; DGAT2 inhibitor; ACC2 inhibitor; glucocorticoid antagonist; acyl-estrogens; lipase inhibitor; fatty acid transporter inhibitor; dicarboxylate transporter inhibitor; glucose transporter inhibitor; serotonin reuptake inhibitors; GLP-1 agonist; amfepramone (diethylpropion), a minorex; amphechloral; amphetamine; axokine; benzphetamine; chlorphentermine; clobenzorex; cloforex; clominorex; clortermine; cyclexedrine; dexfenfluramine; dextroamphetamine; diphemethoxidine, N-ethylamphetamine; fenbutrazate; fenfluramine; fenisorex; fenproporex; fludorex; fluminorex; fluoxetine; furfurylmethylamphetamine; levamfetamine; levophacetoperane; mazindol; mefenorex; metamfepramone; methamphetamine; nalmefene; norpseudoephedrine; pentorex; phendimetrazine; phenmetrazine; phentermine; phenylpropanolamine; phytopharm compound 57; picilorex; metformin; sibutramine; and topiramate, and zonisamide.

The compositions of the present invention are useful in the treatment or prevention of obesity and the following obesity related disorders: overeating; bulimia; hypertension; diabetes, elevated plasma insulin concentrations; insulin resistance; dyslipidemias; hyperlipidemia; endometrial, breast, prostate and colon cancer; osteoarthritis; obstructive sleep apnea; cholelithiasis; gallstones; abnormal heart rhythms; heart arrythmias; myocardial infarction; congestive heart failure; coronary heart disease; sudden death; stroke; polycystic ovarian disease; craniopharyngioma; the Prader-Willi Syndrome; Frohlich's syndrome; GH-deficient subjects; normal variant short stature; Turner's syndrome; metabolic syndrome; and other pathological conditions showing reduced metabolic activity or a decrease in resting energy expenditure as a percentage of total fat-free mass, e.g, children with acute lymphoblastic leukemia. The compositions of the present invention are also useful in the treatment and prevention of male sexual dysfunction, including male erectile dysfunction, and female sexual dysfunction. The compositions of the present invention are further useful in the treatment and prevention of the above disorders while reducing the nausea and emesis side effects caused by administration of the anti-obesity agent or sexual dysfunction therapeutic agent. The invention also relates to pharmaceutical compositions comprising a neurokinin-1 antagonist, and an anti-obesity agent or a sexual dysfunction therapeutic agent, as active ingredients.

The present invention also relates to methods for the treatment and prevention of these conditions, and the use of the compositions of the present invention for manufacture of a medicament useful for treating and preventing these conditions.

The present invention also relates to the treatment or prevention of obesity with a combination of a neurokinin-1 antagonist, and an anti-obesity agent or sexual dysfunction therapeutic which may be administered separately, the invention also relates to combining separate pharmaceutical combinations into a kit form.

### BRIEF DESCRIPTION OF THE DRAWINGS

FIG. 1. Illustrates the effect of the combination of melanocortin 4 agonist, Compound A, and neurokinin-1 antagonist, Compound B, on Food Intake. Ad lib fed ferrets were dosed with vehicle (0.5% MC, 0.5% Tween80, 1 ml/kg po), NK1 antagonist, compound B (10 mg/kg), melanocortin 4 receptor agonist, Compound A (60 mg/kg), or the combination of these two compounds orally in the morning. Food intake was significantly reduced in the NK1 dose group at 24 hours only. Treatment with the combination of NK1 antagonist, Compound B and melanocortin 4 agonist, Compound A resulted in a significantly greater decrease than treatment with Compound A alone. All food intake data were analyzed by two-tailed T-TEST.

FIG. 2. Illustrates the effect of the combination of melanocortin 4 agonist, Compound A, and neurokinin-1 antagonist, Compound B, on Body Weight gain. Ad lib fed ferrets were dosed with vehicle (0.5% MC, 0.5% Tween80, 1 ml/kg po), NK1 antagonist, Compound B (10 mg/kg), melanocortin 4 receptor agonist, Compound A (60 mg/kg), or the combination of these two compounds orally in the morning. Body weight was measured at the time of dosing and at 24 hours. A body weight decrease was observed for the Compound A and the combination group at 24 hours post dose. Treatment with the combination of NK1 agonist Compound B and MC4R agonist Compound A resulted in a significantly greater decrease than treatment with MC4R agonist alone. All body weight data were analyzed by two-tailed T-TEST.

FIG. 3. Illustrates the effect of the combination of melanocortin 4 agonist Compound A, and neurokinin-1 antagonist Compound F on Food Intake. Ad lib fed ferrets were dosed with vehicle (0.25% MC, 0.5% Tween80, 1 ml/kg PO), NK1 antagonist, Compound F (0.3 mg/kg and 1 mg/kg, PO), melanocortin 4 receptor agonist, Compound A (60 mg/kg, PO), or the combinations of Compound F (0.3 mg/kg and 1 mg/kg, PO) and Compound A (60 mg/kg, PO). Food intake was significantly reduced in the combination of Compound F (1 mg/kg, PO) and Compound A (60 mg/kg, PO) dose group at 2 and 24 hours. Treatment with the combinations of NK1 antagonist Compound F, and melanocortin 4 agonist, Compound A resulted in a significantly greater decrease than treatment with Compound A or Compound F alone. All food intake data were analyzed by two-tailed T-TEST.

FIG. 4. Illustrates the effect of the combination of melanocortin 4 agonist, Compound A, and neurokinin-1 antagonist, Compound F, on Body Weight gain. Ad lib fed ferrets were dosed with vehicle (0.25% MC, 0.5% Tween80, 1 ml/kg PO), NK1 antagonist Compound F (0.3 mg/kg and 1 mg/kg, PO), melanocortin 4 receptor agonist, Compound A (60 mg/kg, PO), or the combinations of Compound F (0.3 mg/kg and 1 mg/kg, PO) and Compound A (60 mg/kg, PO). Body weight was measured at the time of dosing and at 24 hours. A body weight decrease was observed for the Compound A and the combination of Compound A and Compound F at 24 hours post dose. Treatment with the combination of NK1 antagonist Compound F and MC4R agonist Compound A resulted in a significantly greater decrease than treatment with the MC4R agonist alone. Treatment with Compound F alone resulted in weight increase relative to vehicle. All body weight data were analyzed by two-tailed T-TEST.

### DETAILED DESCRIPTION OF THE INVENTION

The present invention provides compositions and methods of treatment comprising a neurokinin-1 antagonist, and an anti-obesity agent, in particular a melanocortin 4 agonist, useful in the treatment or prevention of obesity and obesity-related disorders, and useful in reducing nausea and emesis associated with administration of the anti-obesity agent. The present invention further provides compositions and methods of treatment comprising a neurokinin-1 antagonist and a sexual dysfunction therapeutic agent, such as a melanocortin 4 agonist, useful to treat or prevent sexual dysfunction, including male erectile dysfunction and female sexual dysfunction, and in particular useful in reducing nausea and emesis associated with administration of the sexual dysfunction therapeutic agent.

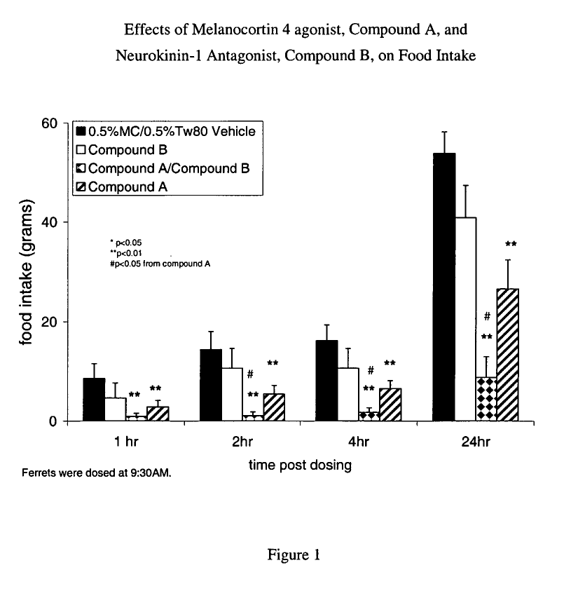

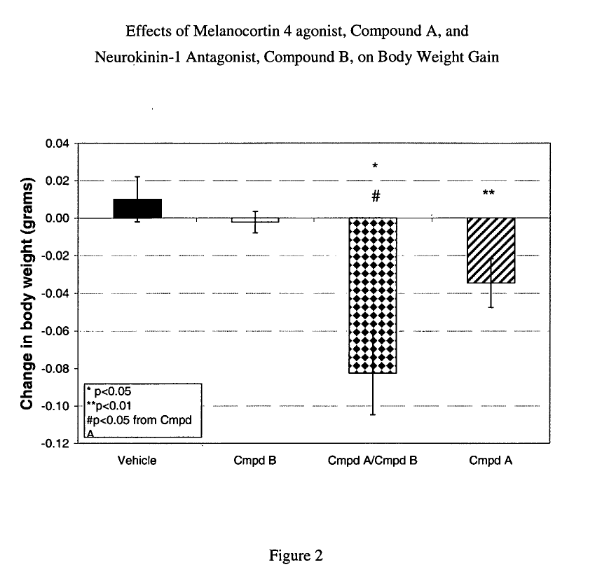

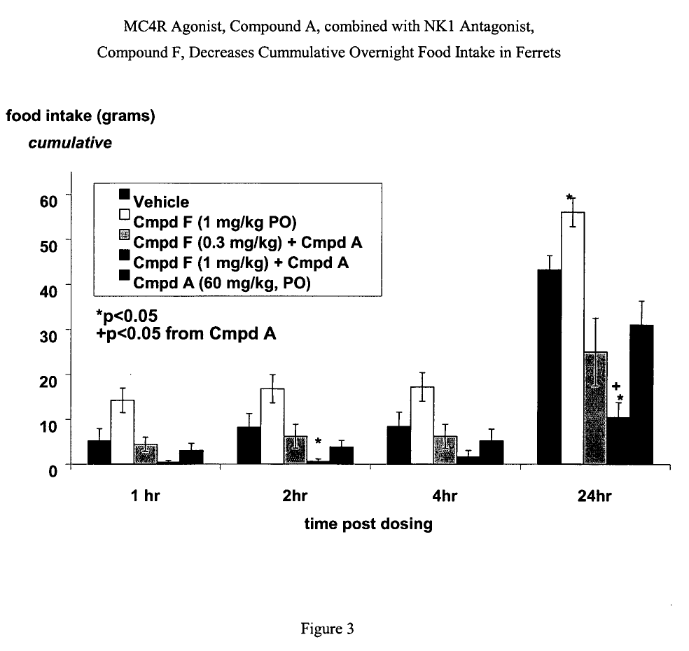

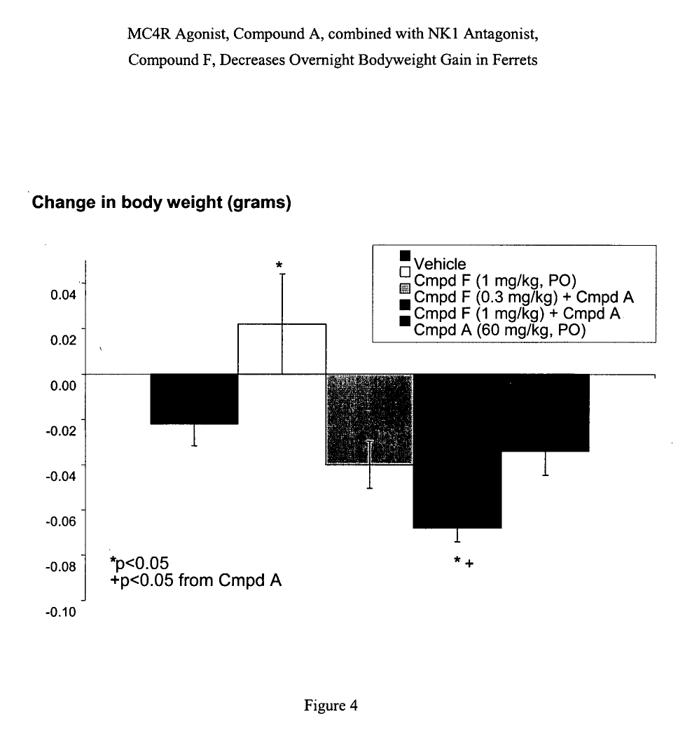

One approach to obesity therapy is reducing food intake (FI) which leads to decreased bodyweight (BW). In the present invention, it was surprisingly found that the combination of a melanocortin 4 receptor agonist, Compound A, and a neurokinin-1 receptor antagonist, Compound B, results in a synergistic decrease in 1, 2, 4 and 24 hour food intake (FI) and a synergistic decrease in body weight (BWT) gain in ferrets (See FIGS. 1 and 2). Specifically, it was found that co-administration of melanocortin 4 receptor agonist, Compound A, and neurokinin-1 receptor antagonist, Compound B, resulted in greater food intake inhibition and a greater body weight decrease in ferrets than the administration of either compound alone (See FIGS. 1 and 2). These studies repeated with a structurally diverse neurokinin-1 antagonist, Compound F, also showed that the combination of melanocortin 4 receptor agonist, Compound A, and neurokinin-1 antagonist, Compound F, results in a greater decrease in 1, 2, 4 and 24 hour food intake (FI) and in a greater decrease in overnight body weight (BWT) gain in ferrets (See FIGS. 3 and 4) than treatment with Compound A or Compound F alone.

Additionally, the studies of the present invention show that co-administration of neurokinin-1 receptor antagonist, Compound B, and melanocortin 4 receptor agonist, Compound A, resulted in a 44% decrease in emesis associated with the administration of the melanocortin 4 agonist (See Table 1).

These studies show that treatment with a neurokinin-1 receptor antagonist and a melanocortin 4 receptor agonist results in greater food intake inhibition and greater weight loss than treatment with either the neurokinin-1 receptor antagonist or the melanocortin 4 agonist alone. These data indicate that a neurokinin-1 receptor antagonist in combination with a melanocortin 4 receptor agonist would be more efficacious in reducing food intake, reducing body weight, reducing body weight gain, maintaining weight loss, and in treating obesity than treatment with either single agent alone. Additionally, these data show that the combination or co-administration of neurokinin-1 antagonist, compound B, with selective melanocortin agonist, Compound A, reduced the emesis caused by administration of the selective the melanocortin 4 agonist.

NK-1 antagonists may also be efficacious in blocking nausea, vomiting and emesis caused by administration of a broad spectrum of anti-obesity agents and sexual dysfunction therapeutic agents. Based on the studies of the present invention, the co-administration or administration of a combination of a neurokinin-1 antagonist and an anti-obesity agent, such as a melanocortin 4 agonist, is useful for treating and preventing obesity; reducing food intake; reducing body weight; prolonging resistance to weight gain and weight regain; and maintaining weight loss, including weight loss due to any cause, including but not limited to diet, drug therapy and exercise. The combination of a neurokinin-1 antagonist and an anti-obesity agent, such as a melanocortin 4 agonist, is useful for treating and preventing the above diseases and disorders while minimizing the emesis side effect caused by administration of the anti-obesity agent, such as the melanocortin 4 receptor agonist.

These studies also suggest that the combination of or co-administration of a neurokinin-1 receptor antagonist and a sexual dysfunction therapeutic agent may be useful to treat or prevent sexual dysfunction, including male erectile dysfunction without the emesis side effect. Specifically, these data indicate that a neurokinin-1 receptor antagonist in combination with a melanocortin 4 receptor agonist would be efficacious in treating and preventing sexual dysfunction while reducing the emesis side effect cause by melanocortin 4 receptor agonists.

The present invention provides compositions comprising a neurokinin-1 antagonist, and an anti-obesity agent, such as a melanocortin 4 agonist, useful in the treatment or prevention of obesity and obesity-related disorders. The present invention also provides compositions comprising a neurokinin-1 antagonist and an anti-obesity agent, such as a melanocortin 4 agonist, useful to reduce food intake and/or bodyweight. The present invention further provides for co-administration of neurokinin-1 antagonist, and an anti-obesity agent, such as a melanocortin 4 agonist, to treat or prevent obesity and obesity related disorders, and reduce food intake and/or bodyweight. The present invention also provides compositions comprising a neurokinin-1 antagonist and a sexual dysfunction therapeutic agent, such as a melanocortin 4 receptor agonist, useful to treat or prevent sexual dysfunction, including male erectile dysfunction and female sexual dysfunction. The present invention also provides compositions comprising a neurokinin-1 antagonist and a sexual dysfunction therapeutic agent, such as a melanocortin 4 agonist, useful to treat or prevent sexual dysfunction, including male erectile dysfunction and female sexual dysfunction. The present invention further provides for co-administration of neurokinin-1 antagonist, and a sexual dysfunction therapeutic agent, such as a melanocortin 4 receptor agonist, to treat or prevent sexual dysfunction.

The present invention provides a method of treating or preventing obesity and obesity-related disorders comprising administration of a therapeutically effective amount of a neurokinin-1 antagonist and a therapeutically effective amount of an anti-obesity agent, such as a melanocortin 4 receptor agonist, to a subject. The present invention provides a method of treating or preventing sexual dysfunction comprising administration of a therapeutically effective amount of a neurokinin-1 antagonist and a therapeutically effective amount of a sexual dysfunction therapeutic agent, such as a melanocortin 4 receptor agonist, to a subject.

In one embodiment of the present invention, the invention comprises a method of treating obesity in a subject comprising administration of: (a) a therapeutically effective amount of a neurokinin-1 antagonist, or a pharmaceutically acceptable salt or ester thereof; and (b) a therapeutically effective amount of an anti-obesity agent, or a pharmaceutically acceptable salt or ester thereof; to a subject in need of such treatment. In a class of this embodiment, the anti-obesity agent is selected from the group consisting of a melanocortin 4 agonist, and pharmaceutically acceptable salts and esters thereof.

In another embodiment of the present invention, the invention comprises a method of treating obesity in a subject comprising administration of: (a) a therapeutically effective amount of a neurokinin-1 antagonist, or a pharmaceutically acceptable salt or ester thereof; and (b) a therapeutically effective amount of an anti-obesity agent, or a pharmaceutically acceptable salt or ester thereof; to a subject in need of such treatment, provided that the anti-obesity agent is not selected from the group consisting of: serotonin agonist, selective serotonin reuptake inhibitor, fluvoxamine, paroxetine, sertraline, a minorex, amphechloral, amphetamine, benzphetamine, p-chloroamphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, fluoxetine, furfurylmethyl-amphetamine, levamfetamine, levophacetoperane, mazindol, mefenorex, metamfepramone, methamphetamine, norpseudoephedrine, pentorex, phendimetrazine, phenmetrazine, phentermine, phenylpropanolamine, picilorex and sibutramine; or a pharmaceutically acceptable salt thereof, to a subject in need of treatment. In a class of this embodiment, the neurokinin-1 antagonist is a CNS penetrant neurokinin-1 antagonist, or a pharmaceutically acceptable salt or ester thereof; to a subject in need of such treatment. In another class of this embodiment, the neurokinin-1 antagonist is an orally active neurokinin-1 antagonist, or a pharmaceutically acceptable salt or ester thereof. In another class of this embodiment, the anti-obesity agent is selected from the group consisting of: CB-1 antagonist/inverse agonist; ghrelin antagonist; H3 antagonist/inverse agonist; MCH1R antagonist; MCH2R agonist/antagonist; MC3R agonist; MC4R agonist; Neuromedin U 1 receptor agonist; Neuromedin U 2 receptor agonist; NPY1 antagonist; NPY2 agonist; NPY4 agonist; NPY5 antagonist; leptin; leptin agonist/modulator; leptin derivatives; opioid antagonist; orexin antagonist; BRS3 agonist; 11β HSD-1 inhibitor, CCK-A agonist; CNTF; CNTF agonist/modulator; CNTF derivative; DP-IV inhibitor; GHS agonist; UCP-1, 2, and 3 activator; β3 agonist; thyroid hormone β agonist; FAS inhibitor; DGAT1 inhibitor; DGAT2 inhibitor; ACC2 inhibitor; glucocorticoid antagonist; acyl-estrogens; lipase inhibitor; fatty acid transporter inhibitor; dicarboxylate transporter inhibitor; glucose transporter inhibitor; GLP-1 agonist; axokine; metformin; phytopharm compound 57; topiramate; and zonisamide; or a pharmaceutically acceptable salt or ester thereof; to a subject in need of such treatment. In another class of this embodiment, the anti-obesity agent is a melanocortin 4 receptor agonist, or a pharmaceutically acceptable salt or ester thereof. In a subclass of this class, the melanocortin 4 receptor agonist has a selective functional activity characterized by an EC₅₀ at least 65-fold lower for the human melanocortin 4 receptor than for the human melanocortin 1 receptor, the melanocortin 2 receptor, the human melanocortin 3 receptor and the human melanocortin 5 receptor. In another subclass of this class, the functional activity of the selective melanocortin 4 receptor agonist is characterized by an EC₅₀ at least 120-fold lower for the human melanocortin 4 receptor than for the human melanocortin 1 receptor. In another subclass of this class, the functional activity of the selective melanocortin 4 receptor agonist is characterized by an EC₅₀ at least 700-fold lower for the human melanocortin 4 receptor than for the human melanocortin 2 receptor. In another subclass of this class, the functional activity of the selective melanocortin 4 receptor agonist is characterized by an EC₅₀ at least 90-fold lower for the human melanocortin 4 receptor than for the human melanocortin 5 receptor.

In another subclass of this class, the selective melanocortin 4 receptor agonist has a binding affinity index (IC₅₀ value) of less than 45 mM at the human melanocortin 4 receptor.

In another embodiment of the present invention, the invention comprises a method of reducing food intake in a subject comprising administration of: (a) a therapeutically effective amount of a neurokinin-1 antagonist, or a pharmaceutically acceptable salt or ester thereof; and (b) a therapeutically effective amount of an anti-obesity agent, or a pharmaceutically acceptable salt or ester thereof; to a subject in need of such treatment. In a class of this embodiment, the anti-obesity agent is selected from the group consisting of a melanocortin 4 agonist, and pharmaceutically acceptable salts and esters thereof.

In another embodiment of the present invention, the invention comprises a method of reducing food intake in a subject comprising administration of: (a) a therapeutically effective amount of a neurokinin-1 antagonist, or a pharmaceutically acceptable salt or ester thereof; and (b) a therapeutically effective amount of an anti-obesity agent selected from the group consisting of: CB-1 antagonist/inverse agonist; ghrelin antagonist; H3 antagonist/inverse agonist; MCH1R antagonist; MCH2R agonist/antagonist; MC3R agonist; MC4R agonist; Neuromedin U 1 receptor agonist; Neuromedin U 2 receptor agonist; NPY1 antagonist; NPY2 agonist; NPY4 agonist; NPY5 antagonist; leptin; leptin agonist/modulator; leptin derivatives; opioid antagonist; orexin antagonist; BRS3 agonist; 11β HSD-1 inhibitor, CCK-A agonist; CNTF; CNTF agonist/modulator; CNTF derivative; DP-IV inhibitor; GHS agonist; UCP-1, 2, and 3 activator; P3 agonist; thyroid hormone β agonist; FAS inhibitor; DGAT1 inhibitor; DGAT2 inhibitor; ACC2 inhibitor; glucocorticoid antagonist; acyl-estrogens; lipase inhibitor; fatty acid transporter inhibitor; dicarboxylate transporter inhibitor; glucose transporter inhibitor; GLP-1 agonist; axokine; metformin; phytopharm compound 57; topiramate; and zonisamide; or a pharmaceutically acceptable salt or ester thereof; to a subject in need of such treatment. In another class of this embodiment, the anti-obesity agent is a melanocortin 4 receptor agonist, or a pharmaceutically acceptable salt or ester thereof, to a subject in need of such treatment.

In another embodiment of the present invention, the invention comprises a method of reducing bodyweight gain in a subject comprising administration of: (a) a therapeutically effective amount of a neurokinin-1 antagonist, or a pharmaceutically acceptable salt or ester thereof; and (b) a therapeutically effective amount of an anti-obesity agent, or a pharmaceutically acceptable salt or ester thereof; to a subject in need of such treatment. In a class of this embodiment, the anti-obesity agent is selected from the group consisting of a melanocortin 4 agonist, and pharmaceutically acceptable salts and esters thereof.

In another embodiment of the present invention, the invention comprises a method of reducing bodyweight in a subject comprising administration of: (a) a therapeutically effective amount of a neurokinin-1 antagonist, or a pharmaceutically acceptable salt or ester thereof; and (b) a therapeutically effective amount of an anti-obesity agent selected from the group consisting of: CB-1 antagonist/inverse agonist; ghrelin antagonist; H3 antagonist/inverse agonist; MCH1R antagonist; MCH2R agonist/antagonist; MC3R agonist; MC4R agonist; Neuromedin U 1 receptor agonist; Neuromedin U 2 receptor agonist; NPY1 antagonist; NPY2 agonist; NPY4 agonist; NPY5 antagonist; leptin; leptin agonist/modulator; leptin derivatives; opioid antagonist; orexin antagonist; BRS3 agonist; 11β HSD-1 inhibitor, CCK-A agonist; CNTF; CNTF agonist/modulator; CNTF derivative; DP-W inhibitor; GHS agonist; UCP-1, 2, and 3 activator; β3 agonist; thyroid hormone β agonist; FAS inhibitor; DGAT1 inhibitor; DGAT2 inhibitor; ACC2 inhibitor; glucocorticoid antagonist; acyl-estrogens; lipase inhibitor; fatty acid transporter inhibitor; dicarboxylate transporter inhibitor; glucose transporter inhibitor; GLP-1 agonist; axokine; metformin; phytopharm compound 57; topiramate; and zonisamide; or a pharmaceutically acceptable salt or ester thereof; to a subject in need of such treatment. In another class of this embodiment, the anti-obesity agent is a melanocortin 4 receptor agonist, or a pharmaceutically acceptable salt or ester thereof.

In another embodiment of the present invention, the invention comprises a method of maintaining weight loss in a subject comprising administration of (a) a therapeutically effective amount of a neurokinin-1 receptor antagonist, and pharmaceutically acceptable salts and esters thereof; and (b) a therapeutically effective amount of an anti-obesity agent; and pharmaceutically acceptable salts and esters thereof; to a subject in need of such treatment. In a class of this embodiment, the anti-obesity agent is selected from the group consisting of a melanocortin 4 agonist, and pharmaceutically acceptable salts and esters thereof.

In another embodiment of the present invention, the invention comprises a method for reducing nausea or emesis associated with the administration of an anti-obesity agent for the treatment or prevention of obesity or an obesity related disorder in a subject comprising administration of (a) a therapeutically effective amount of a neurokinin-1 receptor antagonist, and pharmaceutically acceptable salts and esters thereof; and (b) a therapeutically effective amount of an anti-obesity agent; and pharmaceutically acceptable salts and esters thereof; to a subject in need of such treatment. In a class of this embodiment, the anti-obesity agent is selected from the group consisting of a melanocortin 4 agonist, and pharmaceutically acceptable salts and esters thereof.

In another embodiment of the present invention, the invention comprises a method for reducing nausea or emesis associated with the administration of a melanocortin 4 receptor agonist for the treatment or prevention of obesity, or an obesity related disorder in a subject comprising administration of: (a) a therapeutically effective amount of a neurokinin-1 antagonist, or a pharmaceutically acceptable salt or ester thereof; and (b) a therapeutically effective amount of a melanocortin 4 receptor agonist, or a pharmaceutically acceptable salt or ester thereof; to a subject in need of such treatment.

In another embodiment of the present invention, the invention comprises a method of treating an obesity-related disorder in a subject comprising administration of: (a) a therapeutically effective amount of a neurokinin-1 antagonist, or a pharmaceutically acceptable salt or ester thereof; and (b) a therapeutically effective amount of an anti-obesity agent, or a pharmaceutically acceptable salt or ester thereof; to a subject in need of such treatment. In a class of this embodiment, the anti-obesity agent is selected from the group consisting of a melanocortin 4 agonist, and pharmaceutically acceptable salts and esters thereof.

In another embodiment of the present invention, the invention comprises a method of treating an obesity-related disorder in a subject comprising administration of: (a) a therapeutically effective amount of a neurokinin-1 antagonist, or a pharmaceutically acceptable salt or ester thereof; and (b) a therapeutically effective amount of an anti-obesity agent selected from the group consisting of: CB-1 antagonist/inverse agonist; ghrelin antagonist; H3 antagonist/inverse agonist; MCH1R antagonist; MCH2R agonist/antagonist; MC3R agonist; MC4R agonist; Neuromedin U 1 receptor agonist; Neuromedin U 2 receptor agonist; NPY1 antagonist; NPY2 agonist; NPY4 agonist; NPY5 antagonist; leptin; leptin agonist/modulator; leptin derivatives; opioid antagonist; orexin antagonist; BRS3 agonist; 11β HSD-1 inhibitor, CCK-A agonist; CNTF; CNTF agonist/modulator; CNTF derivative; DP-IV inhibitor; GHS agonist; UCP-1, 2, and 3 activator; β agonist; thyroid hormone β agonist; FAS inhibitor; DGAT1 inhibitor; DGAT2 inhibitor; ACC2 inhibitor; glucocorticoid antagonist; acyl-estrogens; lipase inhibitor; fatty acid transporter inhibitor; dicarboxylate transporter inhibitor; glucose transporter inhibitor; GLP-1 agonist; axokine; metformin; phytopharm compound 57; topiramate; and zonisamide; or a pharmaceutically acceptable salt or ester thereof; to a subject in need of such treatment. In another class of this embodiment, the anti-obesity agent is a melanocortin 4 receptor agonist, or a pharmaceutically acceptable salt or ester thereof. In another class of this embodiment, the obesity-related disorder is selected from: overeating; bulimia; hypertension; diabetes, elevated plasma insulin concentrations; insulin resistance; dyslipidemia; hyperlipidemia; endometrial, breast, prostate and colon cancer; osteoarthritis; obstructive sleep apnea; cholelithiasis; gallstones; coronary heart disease; abnormal heart rhythms; heart arrythmias; myocardial infarction; polycystic ovarian disease; craniopharyngioma; the Prader-Willi Syndrome; Frohlich's syndrome; GH-deficient subjects; normal variant short stature; Turner's syndrome; metabolic syndrome; and acute lymphoblastic leukemia. In another class of this embodiment, the obesity-related disorder is diabetes. In another class of this embodiment, the obesity-related disorder is metabolic syndrome.

In another embodiment of the present invention, the invention comprises a method of preventing obesity or an obesity related disorder in a subject at risk for obesity or an obesity-related disorder comprising administration to said subject (a) a prophylactically effective amount of a neurokinin-1 antagonist, or a pharmaceutically effective salt or ester thereof; and (b) a prophylactically effective amount of an anti-obesity agent, or a pharmaceutically effective salt or ester thereof.

In another embodiment of the present invention, the invention comprises a method of preventing obesity or an obesity related disorder in a subject at risk for obesity or an obesity-related disorder comprising administration to said subject (a) a prophylactically effective amount of a neurokinin-1 antagonist, or a pharmaceutically effective salt or ester thereof; and (b) a prophylactically effective amount of an anti-obesity agent selected from the group consisting of: CB-1 antagonist/inverse agonist; ghrelin antagonist; H3 antagonist/inverse agonist; MCH1R antagonist; MCH2R agonist/antagonist; MC3R agonist; MC4R agonist; Neuromedin U 1 receptor agonist; Neuromedin U 2 receptor agonist; NPY1 antagonist; NPY2 agonist; NPY4 agonist; NPY5 antagonist; leptin; leptin agonist/modulator; leptin derivatives; opioid antagonist; orexin antagonist; BRS3 agonist; 11β HSD-1 inhibitor, CCK-A agonist; CNTF; CNTF agonist/modulator; CNTF derivative; DP-IV inhibitor; GHS agonist; UCP-1, 2, and 3 activator; β3 agonist; thyroid hormone β agonist; FAS inhibitor; DGAT1 inhibitor; DGAT2 inhibitor; ACC2 inhibitor; glucocorticoid antagonist; acyl-estrogens; lipase inhibitor; fatty acid transporter inhibitor; dicarboxylate transporter inhibitor; glucose transporter inhibitor; GLP-1 agonist; axokine; metformin; phytopharm compound 57; topiramate; and zonisamide; or a pharmaceutically effective salt or ester thereof. In a class of this embodiment, the anti-obesity agent is selected from the group consisting of a melanocortin 4 agonist, and pharmaceutically acceptable salts and esters thereof.

In another embodiment of the present invention, the invention comprises a method of treating sexual dysfunction in a subject in need thereof comprising administration of (a) a therapeutically effective amount of a neurokinin-1 antagonist, and pharmaceutically acceptable salts and esters thereof; and (b) a therapeutically effective amount of a sexual dysfunction therapeutic agent, and pharmaceutically acceptable salts and esters thereof.

In another embodiment of the present invention, the invention comprises a method of treating sexual dysfunction in a subject in need thereof comprising administration of (a) a therapeutically effective amount of a neurokinin-1 antagonist, or a pharmaceutically acceptable salt or ester thereof; and (b) a therapeutically effective amount of a sexual dysfunction therapeutic agent, or a pharmaceutically acceptable salt or ester thereof. In a class of this embodiment, the sexual dysfunction therapeutic agent is selected from the group consisting of: a type V cyclic-GMP-selective phosphodiesterase inhibitor; an α₁-adrenergic receptor antagonist; an α₂-adrenergic receptor antagonist; a dopamine-2 receptor agonist; a dopamine-3 receptor agonist; a dopamine-4 receptor agonist; an oxytocin receptor antagonist; a serotonergic 5HT1B agonist; a serotonergic 5HT2C agonist; MT-II; PT-141; PT-14; apomorphine; and sildenifil; and pharmaceutically acceptable salts and esters thereof, to a subject in need of such treatment. In a class of this embodiment, the sexual dysfunction therapeutic agent is a melanocortin 4 receptor agonist, or a pharmaceutically acceptable salt or ester thereof. In another class of this embodiment, the treatment of sexual dysfunction is the treatment of sexual dysfunction is the treatment of female sexual dysfunction.

In another embodiment of the present invention, the invention comprises a method for reducing nausea or emesis associated with the administration of a sexual dysfunction therapeutic agent for the treatment or prevention of sexual dysfunction in a subject in need thereof comprising administration of (a) a therapeutically effective amount of a neurokinin-1 antagonist, or a pharmaceutically acceptable salt or ester thereof; and (b) a therapeutically effective amount of a sexual dysfunction therapeutic agent, or a pharmaceutically acceptable salt or ester thereof.

In another embodiment of the present invention, the invention comprises a method for reducing nausea or emesis associated with the administration of a melanocortin 4 receptor agonist for the treatment or prevention of sexual dysfunction in a subject comprising administration of: (a) a therapeutically effective amount of a neurokinin-1 antagonist, or a pharmaceutically acceptable salt or ester thereof; and (b) a therapeutically effective amount of a melanocortin 4 receptor agonist, or a pharmaceutically acceptable salt or ester thereof; to a subject in need of such treatment.

In another embodiment of the present invention, the invention comprises a method of preventing sexual dysfunction in a subject at risk for sexual dysfunction comprising administration to said subject (a) a prophylactically effective amount of a neurokinin-1 receptor antagonist, or a pharmaceutically effective salt or ester thereof; and (b) a prophylactically effective amount of a sexual dysfunction therapeutic agent, or a pharmaceutically acceptable salt or ester thereof.

In another embodiment of the present invention, the invention comprises a method of preventing sexual dysfunction in a subject in subject at risk for sexual dysfunction comprising administration to said subject (a) a prophylactically effective amount of a neurokinin-1 receptor antagonist, or a pharmaceutically effective salt or ester thereof; and (b) a prophylactically effective amount of a sexual dysfunction therapeutic agent selected from the group consisting of: a type V cyclic-GMP-selective phosphodiesterase inhibitor; an α₁-adrenergic receptor antagonist; an α₂-adrenergic receptor antagonist; a dopamine-2 receptor agonist; a dopamine-3 receptor agonist; a dopamine-4 receptor agonist; an oxytocin receptor antagonist; a serotonergic 5HT1B agonist; a serotonergic 5HT2C agonist; MT-II; PT-141; PT-14; apomorphine; and sildenifil, or a pharmaceutically acceptable salt or ester thereof. In a class of this embodiment, the sexual dysfunction therapeutic agent is a melanocortin 4 receptor agonist, or a pharmaceutically acceptable salt or ester thereof. In another class of this embodiment, the treatment of sexual dysfunction is the treatment of male erectile dysfunction. In another class of this embodiment, the treatment of sexual dysfunction is the treatment of female sexual dysfunction.

In another embodiment of the present invention, the invention comprises a product comprising a neurokinin-1 antagonist, or a pharmaceutically acceptable salt or ester thereof; and an anti-obesity agent, or a pharmaceutically acceptable salt or ester thereof, as a combined preparation for simultaneous, separate or sequential use in the treatment of obesity or an obesity disorder. In another embodiment of the present invention, the invention comprises a product comprising a neurokinin-1 antagonist, or a pharmaceutically acceptable salt or ester thereof; and an anti-obesity agent, or a pharmaceutically acceptable salt or ester thereof, provided that the anti-obesity agent is not selected from the group consisting of: selective serotonin reuptake inhibitor, fluvoxamine, paroxetine, sertraline, a minorex, amphechloral, amphetamine, benzphetamine, p-chloroamphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, fluoxetine, furfurylmethyl-amphetamine, levamfetamine, levophacetoperane, mazindol, mefenorex, metamfepramone, methamphetamine, norpseudoephedrine, pentorex, phendimetrazine, phenmetrazine, phentermine, phenylpropanolamine, picilorex and sibutramine; and pharmaceutically acceptable salts thereof, as a combined preparation for simultaneous, separate or sequential use in the treatment of obesity or an obesity disorder. In a class of this embodiment, the neurokinin-1 antagonist is 2-(R)-(1-(R)-(3,5-bis(trifluoromethyl)phenyl)-ethoxy)-4-(5-(dimethylamino)methyl-1,2,3-triazol-4-yl)methyl-3-(S)-(4-fluorophenyl)morpholine, or a pharmaceutically acceptable salt or ester thereof. In a subclass of this class, the anti-obesity agent is a melanocortin 4 agonist, or a pharmaceutically acceptable salt or ester thereof. In a subclass of this class, the melanocortin 4 agonist is Compound A or a pharmaceutically acceptable salt or ester thereof.

In another embodiment of the present invention, the invention comprises a product comprising a neurokinin-1 antagonist, or a pharmaceutically acceptable salt or ester thereof; and a sexual dysfunction therapeutic agent, or a pharmaceutically acceptable salt or ester thereof, as a combined preparation for simultaneous, separate or sequential use in the treatment of sexual dysfunction.

In another class of the embodiments of the present invention, the neurokinin-1 antagonist is a CNS penetrant neurokinin-1 antagonist, or a pharmaceutically acceptable salt or ester thereof. In another class of this embodiment, the neurokinin-1 antagonist is an orally active neurokinin-1 antagonist, or a pharmaceutically acceptable salt or ester thereof. In another class of the embodiments of the present invention, the neurokinin-1 antagonist is a compound selected from the group consisting of:
(1) (±)-(2R3R,2S3S)—N-{[2-cyclopropoxy-5-(trifluoromethoxy)-phenyl]methyl}-2-phenylpiperidin-3-amine;
(2) 2-(S)-(3,5-bis(trifluoromethyl)benzyloxy)-3(S)-(4-fluorophenyl)-4-(3-(5-oxo-1H,4H-1,2,4-triazolo)methyl)morpholine;
(3) 2-(R)-(1-(R)-(3,5-bis(trifluoromethyl)phenyl)ethoxy)-4-(3-(5-oxo-1H,4H-1,2,4-triazolo)methyl)-3-(S)-phenyl-morpholine;
(4) 2-(S)-(3,5-bis(trifluoromethyl)benzyloxy)-4-(3-(5-oxo-1H,4H-1,2,4-triazolo)methyl)-3-(S)-phenyl-morpholine;
(5) 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine;
(6) 2-(R)-(1-(R)-(3,5-bis(trifluoromethyl)phenyl)ethoxy)-4-(5-(N,N-dimethylamino)methyl-1,2,3-triazol-4-yl)methyl-3-(S)-phenylmorpholine;
(7) 2-(R)-(1-(R)-(3,5-bis(trifluoromethyl)phenyl)ethoxy)-4-(5-(N,N-dimethylamino)methyl-1,2,3-triazol-4-yl)methyl-3-(S)-(4-fluorophenyl)morpholine;
(8) (3S,5R,6S)-3-[2-cyclopropoxy-5-(trifluoromethoxy)phenyl]-6-phenyl-1-oxa-7-aza-spiro[4.5]decane;
(9) (3R,5R,6S)-3-[2-cyclopropoxy-5-(trifluoromethoxy)phenyl]-6-phenyl-1-oxa-7-aza-spiro[4.5]decane;
(10) 2-(R)-(1-(S)-(3,5-bis(trifluoromethyl)phenyl)-2-hydroxyethoxy)-3-(S)-(4-fluorophenyl)-4-(1,2,4-triazol-3-yl)methylmorpholine;
(11) 2-(R)-(1-(R)-(3,5-bis(trifluoromethyl)phenyl)ethoxy)-3-(S)-(4-fluorophenyl)-4-(3-(4-monophosphoryl-5-oxo-1H-1,2,4-triazolo)methyl)morpholine;
(12) 2-(R)-(1-(R)-(3,5-bis(trifluoromethyl)phenyl)ethoxy)-3-(S)-(4-fluorophenyl)-4-(3-(1-monophosphoryl-5-oxo-1H-1,2,4-triazolo)methyl)morpholine;
(13) 2-(R)-(1-(R)-(3,5-bis(trifluoromethyl)phenyl)ethoxy)-3-(S)-(4-fluorophenyl)-4-(3-(2-monophosphoryl-5-oxo-1H-1,2,4-triazolo)methyl)morpholine;
(14) 2-(R)-(1-(R)-(3,5-bis(trifluoromethyl)phenyl)ethoxy)-3-(S)-(4-fluorophenyl)-4-(3-(5-oxyphosphoryl-1H-1,2,4-triazolo)methyl)morpholine;
(15) 2-(S)-(1-(R)-(3,5-bis(trifluoromethyl)phenyl)ethoxy)-3-(S)-(4-fluorophenyl)-4-(3-(1-monophosphoryl-5-oxo-4H-1,2,4-triazolo)methyl)morpholine;
(16) 2-(R)-(1-(R)-(3,5-bis(trifluoromethyl)phenyl)ethoxy)-4-(4-N,N-dimethylaminobut-2-yn-yl)-3-(S)-(4-fluorophenyl)morpholine;
   or a pharmaceutically acceptable salt or ester thereof. In another class of this embodiment, the neurokinin-1 antagoinst is selected from the group consisting of: 2-(R)-(1-(R)-(3,5-bis(trifluoromethyl)phenyl)ethoxy)-4-(5-(dimethylamino)methyl-1,2,3-triazol-4-yl)methyl-3-(S)-(4-fluorophenyl)morpholine, and pharmaceutically acceptable salts, esters and tautomers thereof.

In another class of the embodiments of the present invention, the neurokinin-1 antagonist is selected from the group consisting of compound F

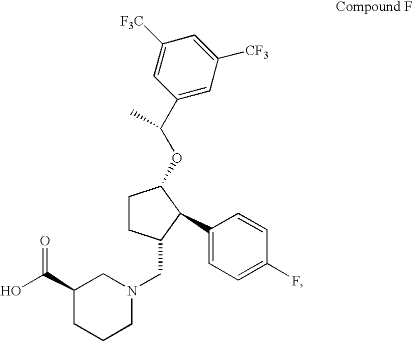

and pharmaceutically acceptable salts and esters thereof. Compound F, and its preparation are disclosed in U.S. Pat. No. 5,750,549.

In another class of the embodiments of the present invention, the neurokinin-1 antagonist is selected from the group consisting of compound F, and pharmaceutically acceptable salts and esters thereof; and the anti-obesity agent is a melanocortin 4 agonist selected from the group consisting of Compound A, and pharmaceutically acceptable salts and esters thereof.

In another class of the embodiments of the present invention, the anti-obesity agent selected from the group consisting of: CB-1 antagonist/inverse agonist; ghrelin antagonist; H3 antagonist/inverse agonist; MCH1R antagonist; MCH2R agonist/antagonist; MC3R agonist; MC4R agonist; neuromedin U 1 receptor agonist; neuromedin U 2 receptor agonist; NPY1 antagonist; NPY2 agonist; NPY4 agonist; NPY5 antagonist; leptin; leptin agonist/modulator; leptin derivatives; opioid antagonist; orexin antagonist; BRS3 agonist; 11β HSD-1 inhibitor, CCK-A agonist; CNTF; CNTF agonist/modulator; CNTF derivative; DP-IV inhibitor; GHS agonist; UCP-1, 2, and 3 activator; β3 agonist; thyroid hormone β agonist; FAS inhibitor; DGAT1 inhibitor; DGAT2 inhibitor; ACC2 inhibitor; glucocorticoid antagonist; acyl-estrogens; lipase inhibitor; fatty acid transporter inhibitor; dicarboxylate transporter inhibitor; glucose transporter inhibitor; GLP-1 agonist; axokine; metformin; nalmefene; phytopharm compound 57; topiramate; and zonisamide; and pharmaceutically acceptable salts and esters thereof.

In another class of the embodiments of the present invention, the anti-obesity agent is selected from the group consisting of: CB-1 antagonist/inverse agonist; ghrelin antagonist; H3 antagonist/inverse agonist; MCH1R antagonist; MCH2R agonist/antagonist; MC4R agonist; NPY1 antagonist; NPY2 agonist; NPY5 antagonist; leptin, leptin agonist/modulator; leptin derivatives; opioid antagonist; BRS3 agonist; 11β HSD-1 inhibitor, CCK-A agonist; and pharmaceutically acceptable salts and esters thereof.

In another embodiment of the present invention, the anti-obesity agent is selected from the group consisting of: a CB-1 antagonist/inverse agonist; a ghrelin antagonist; a H3 antagonist/inverse agonist; a MCH1R antagonist; a MCH2R agonist/antagonist; a MC4R agonist; a NPY1 antagonist; a NPY2 agonist; a NPY5 antagonist; a BRS3 agonist; a 11β HSD-1 inhibitor, and a CCK-A agonist; and pharmaceutically acceptable salts and esters thereof.

In another class of the embodiments of the present invention, the anti-obesity agent is an acyl-estrogen selected from oleoyl-estrone, or a pharmaceutically acceptable salt or ester thereof. In another class of the embodiments of the present invention, the anti-obesity agent is a CNTF derivative selected from axokine, or a pharmaceutically acceptable salt or ester thereof. In another class of the embodiments of the present invention, the anti-obesity agent is a lipase inhibitor selected from orlistat, or a pharmaceutically acceptable salt or ester thereof. In another class of the embodiments, the anti-obesity agent is selected from leptin, or a pharmaceutically acceptable salts or ester thereof. In another class of the embodiments of the present invention, the anti-obesity agent is an opioid antagonist selected from nalmefene, or a pharmaceutically acceptable salt or ester thereof. In another class of the embodiments of the present invention, the anti-obesity agent is a CB-1 inverse agonist, or a pharmaceutically acceptable salt or ester thereof. In a subclass of this class, the CB-1 inverse agonist is selected from the group consisting of rimonabant, and pharmaceutically acceptable salts and esters thereof. In another class of the embodiments of the present invention, the anti-obesity agent is a NPY2 agonist selected from the group consisting of PYY, PYY₃₋₃₆, and pharmaceutically acceptable salts and esters thereof.

In another class of the embodiments of the present invention, the anti-obesity agent is selected from the group consisting of: CB-1 antagonist/inverse agonist; ghrelin antagonist; H3 antagonist/inverse agonist; MCH1R antagonist; MCH2R agonist/antagonist; MC4R agonist; NPY1 antagonist; NPY2 agonist; NPY5 antagonist; DP-IV inhibitor; BRS3 agonist; 11β HSD-1 inhibitor, and CCK-A agonist; and pharmaceutically acceptable salts and esters thereof.

In another class of the embodiments of the present invention, the anti-obesity agent is selected from the group consisting of: orlistat, sibutramine, phentermine, topiramate, and pharmaceutically acceptable salts and esters thereof.

In another class of the embodiments of the present invention, the anti-obesity agent is selected from the group consisting of a melanocortin 4 receptor agonist, and pharmaceutically acceptable salts or esters thereof. In a subclass of this class, the melanocortin 4 agonist is orally active. In another subclass of this class, the melanocortin 4 agonist is a selective melanocortin 4 agonist. In another subclass of this class, the anti-obesity agent is a melanocortin 4 receptor agonist selected from the group consisting of compounds of Formulas I and II, and pharmaceutically acceptable salts and esters thereof. In another subclass of this class, the anti-obesity agent is a melanocortin 4 agonist selected from the group consisting of Compound A, and pharmaceutically acceptable salts and esters thereof.

In another class of the embodiments of the present invention, the neurokinin-1 antagonist is selected from the group consisting of compound B, and pharmaceutically acceptable salts and esters thereof; and the anti-obesity agent is a melanocortin 4 agonist selected from the group consisting of Compound A, and pharmaceutically acceptable salts and esters thereof.

In another class of the embodiments of the present invention, the neurokinin-1 antagonist is 2-(R)-(1-(1R)-(3,5-bis(trifluoromethyl)phenyl)ethoxy)-4-(5-(dimethylamino)methyl-1,2,3-triazol-4-yl) methyl-3-(S)-(4-fluorophenyl)morpholine, or a pharmaceutically acceptable salt, ester or tautomer thereof; and the anti-obesity agent is

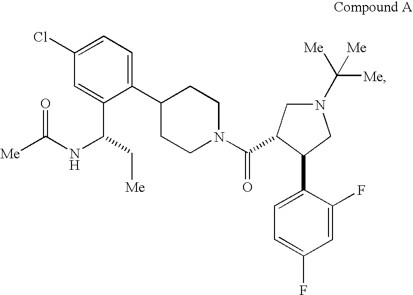

or a pharmaceutically acceptable salt or ester thereof.

In another class of the embodiments of the present invention, the subject in need of treatment or prevention is suffering from nausea and/or emesis.

In another class of the embodiments of the present invention, the obesity-related disorder is selected from: overeating; bulimia; hypertension; diabetes, elevated plasma insulin concentrations; insulin resistance; dyslipidemia; hyperlipidemia; endometrial, breast, prostate and colon cancer; osteoarthritis; obstructive sleep apnea; cholelithiasis; gallstones; coronary heart disease; abnormal heart rhythms; heart arrythmias; myocardial infarction; polycystic ovarian disease; craniopharyngioma; the Prader-Willi Syndrome; Frohlich's syndrome; GH-deficient subjects; normal variant short stature; Turner's syndrome; metabolic syndrome; and acute lymphoblastic leukemia. In a subclass of this class, the obesity-related disorder is diabetes. In another subclass of this class, the obesity-related disorder is metabolic syndrome.

In another class of the embodiments of the present invention, the subject in need of treatment or prevention is suffering from emesis. In a subclass of this class, the emesis is caused by the treatment with an anti-obesity agent or a sexual dysfunction therapeutic agent. In another subclass of this class, the emesis is caused by the treatment with a melanocortin 4 agonist.

In another class of the embodiments of the present invention, the sexual dysfunction therapeutic agent is selected from the group consisting of:

(1) a type V cyclic-GMP-selective phosphodiesterase inhibitor;

(2) an α₁-adrenergic receptor antagonist; and

(3) an α₂-adrenergic receptor antagonist;

(4) a dopamine-2 receptor agonist;

(5) a dopamine-3 receptor agonist;

(6) a dopamine-4 receptor agonist;

(7) an oxytocin receptor antagonist;

(8) a serotonergic 5HT1B agonist; and

(9) a serotonergic 5HT2C agonist;

and pharmaceutically acceptable salts and esters thereof.

In a subclass of this class, the sexual dysfunction therapeutic agent is selected from the group consisting of: a type V cyclic-GMP-selective phosphodiesterase inhibitor; an α₁-adrenergic receptor antagonist; an α₂-adrenergic receptor antagonist; a dopamine-2 receptor agonist; a dopamine-3 receptor agonist; a dopamine-4 receptor agonist; and an oxytocin receptor antagonist; and pharmaceutically acceptable salts and esters thereof. In another subclass of this class, the sexual dysfunction therapeutic agent is selected from the group consisting of: a type V cyclic-GMP-selective phosphodiesterase inhibitor; and pharmaceutically acceptable salts and esters thereof.

In another class of the embodiments of the present invention, the sexual dysfunction therapeutic agent is selected from the group consisting of MT-II; PT-141; PT-14; apomorphine; and sildenifil; and pharmaceutically acceptable salts and esters thereof.

In another class of the embodiments of the present invention, the sexual dysfunction therapeutic agent is selected from the group consisting of a melanocortin 4 receptor agonist, and pharmaceutically acceptable salts or esters thereof. In a subclass of this class, the melanocortin 4 agonist is orally active.

In another subclass of this class, the melanocortin 4 agonist is a selective melanocortin 4 agonist. In another subclass of this class, the sexual agent dysfunction therapeutic agent is a melanocortin 4 agonist selected from the group consisting of compounds of Formulas I and II, and pharmaceutically acceptable salts and esters thereof. In another class of the embodiments of the present invention, the sexual dysfunction therapeutic agent is a melanocortin 4 agonist selected from the group consisting of Compound A, and pharmaceutically acceptable salts and esters thereof.

In another class of the embodiments of the present invention, the neurokinin-1 antagonist is 2-(R)-(1-(R)-(3,5-bis(trifluoromethyl)phenyl)ethoxy)-4-(5-(dimethylamino)methyl-1,2,3-triazol-4-yl)methyl-3-(S)-(4-fluorophenyl)morpholine, or a pharmaceutically acceptable salt, ester or tautomer thereof; and the sexual dysfunction therapeutic agent is a melanocortin 4 agonist selected from the group consisting of

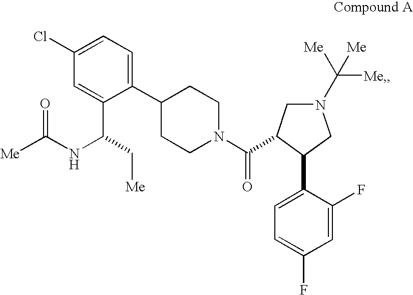

and pharmaceutically acceptable salts and esters thereof.

In another class of the embodiments of the present invention, the sexual dysfunction is male erectile dysfunction.

In another class of the embodiments of the present invention, the sexual dysfunction is female erectile dysfunction. In a subclass of this class, female sexual dysfunction is selected from the group consisting of: female arousal dysfunction, female orgasmic disorder, hypoactive sexual desire disorder, and sexual pain disorder.

In another class of the embodiments of the present invention, the subject in need of treatment or prevention is suffering from emesis. In another subclass of this class, the emesis is caused by the treatment with a melanocortin 4 agonist.

In another class of the embodiments of the present invention, the invention is directed to a method of treating or preventing obesity, an obesity-related disorder, or sexual dysfunction comprising administering to a subject a therapeutically effective amount of a neurokinin-1 antagonist, or a pharmaceutically acceptable salt or ester thereof, and a therapeutically effective amount of a selective melanocortin 4 receptor agonist, or a pharmaceutically acceptable salt thereof, wherein the functional activity of the melanocortin 4 receptor agonist is characterized by an EC₅₀ at least 14-fold more selective for the human melanocortin 4 receptor than for the human melanocortin 1 receptor, human melanocortin 2 receptor, the human melanocortin 3 receptor and the human melanocortin 5 receptor. In another embodiment of the present invention, the invention is directed to a method of treating or preventing obesity, an obesity-related disorder or sexual dysfunction comprising administering to a subject a therapeutically effective amount of a neurokinin-1 antagonist, or a pharmaceutically acceptable salt or ester thereof, and a therapeutically effective amount of a selective melanocortin 4 receptor agonist, or a pharmaceutically acceptable salt thereof, wherein the functional activity of the melanocortin 4 receptor agonist is characterized by an EC₅₀ at least 65-fold more selective for the human melanocortin 4 receptor than for the human melanocortin 1 receptor, human melanocortin 2 receptor, the human melanocortin 3 receptor and the human melanocortin 5 receptor. In another embodiment of the present invention, the invention is directed to a method of treating or preventing obesity, an obesity-related disorder or sexual dysfunction comprising administering to a subject a therapeutically effective amount of a neurokinin-1 receptor antagonist, or a pharmaceutically acceptable salt or ester thereof, and a therapeutically effective amount of a selective melanocortin 4 receptor agonist, or a pharmaceutically acceptable salt thereof, wherein the functional activity of the melanocortin 4 receptor agonist is characterized by an EC₅₀ at least 200-fold more selective for the human melanocortin 4 receptor than for the human melanocortin 1 receptor, human melanocortin 2 receptor, the human melanocortin 3 receptor and the human melanocortin 5 receptor.

In a class of the embodiments of the present invention, the functional activity of the melanocortin 4 agonist is characterized by an EC₅₀ at least 14-fold more selective for the human melanocortin 4 receptor than for the human melanocortin 1 receptor. In another class of the embodiments of the present invention, the functional activity of the melanocortin 4 agonist is characterized by an EC₅₀ at least 120-fold more selective for the human melanocortin 4 receptor than for the human melanocortin 1 receptor.

In another class of the embodiments of the present invention, the functional activity of the melanocortin 4 agonist is characterized by an EC₅₀ at least 100-fold more selective for the human melanocortin 4 receptor than for the human melanocortin 2 receptor. In another class of the embodiments of the present invention, the functional activity of the melanocortin 4 agonist is characterized by an EC₅₀ at least 700-fold more selective for the human melanocortin 4 receptor than for the human melanocortin 2 receptor.

In another class of the embodiments of the present invention, the functional activity of the melanocortin 4 agonist is characterized by an EC₅₀ at least 30-fold more selective for the human melanocortin 4 receptor than for the human melanocortin 3 receptor. In another class of the embodiments of the present invention, the functional activity of the melanocortin 4 agonist is characterized by an EC₅₀ at least 65-fold more selective for the human melanocortin 4 receptor than for the human melanocortin 3 receptor.

In another class of the embodiments of the present invention, the functional activity of the melanocortin 4 agonist is characterized by an EC₅₀ at least 50-fold more selective for the human melanocortin 4 receptor than for the human melanocortin 5 receptor. In another class of the embodiments of the present invention, the functional activity of the melanocortin 4 agonist is characterized by an EC₅₀ at least 90-fold more selective for the human melanocortin 4 receptor than for the human melanocortin 5 receptor.

In another embodiment of the present invention, the invention is directed to a method of treating or preventing obesity, an obesity-related disorder or sexual dysfunction comprising administering to a subject a therapeutically effective amount of a neurokinin-1 antagonist, or a pharmaceutically acceptable salt or ester thereof, and a therapeutically effective amount of a melanocortin 4 receptor agonist, or a pharmaceutically acceptable salt thereof, wherein the binding affinity index of the melanocortin 4 receptor agonist is characterized by an IC₅₀ value at least 35-fold more selective for the human melanocortin 4 receptor than for the human melanocortin 1 receptor, the human melanocortin 3 receptor and the human melanocortin 5 receptor.

In a class of this embodiment, the binding affinity index of the melanocortin 4 agonist is characterized by an IC₅₀ value at least 125-fold more selective for the human melanocortin 4 receptor than for the human melanocortin 1 receptor. In another class of this embodiment, the binding affinity index of the melanocortin 4 agonist is characterized by an IC₅₀ value at least 35-fold more selective for the human melanocortin 4 receptor than for the human melanocortin 3 receptor. In another class of this embodiment, the binding affinity index of the melanocortin 4 agonist is characterized by an IC₅₀ value at least 160-fold more selective for the human melanocortin 4 receptor than for the human melanocortin 3 receptor. In another class of this embodiment, the binding affinity index of the melanocortin 4 agonist is characterized by an IC₅₀ value at least 12-fold more selective for the human melanocortin 4 receptor than for the human melanocortin 5 receptor. In another class of this embodiment, the binding affinity index of the melanocortin 4 agonist is characterized by an IC₅₀ value at least 35-fold more selective for the human melanocortin 4 receptor than for the human melanocortin 5 receptor.

Administration may be in a fixed combination or via co-administration of separate compositions or formulations. The present invention also relates to pharmaceutical compositions, and medicaments useful for carrying out these methods.

The present invention is also concerned with treatment of these conditions, and the use of the compositions of the present invention for manufacture of a medicament useful for treating these conditions.

The methods and compositions of the present invention comprise an anti-obesity agent. The anti-obesity agents useful in the compositions of the present invention may be any agent useful to decrease food intake known in the art. The anti-obesity agent may be peptidal or non-peptidal in nature; however, the use of a non-peptidal agent is preferred. For convenience, the use of an orally active anti-obesity agent is also preferred.

The anti-obesity agents useful in the compositions of the present invention is selected from the group consisting of: CB-1 antagonist/inverse agonist; ghrelin antagonist; H3 antagonist/inverse agonist; MCH1R antagonist; MCH2R agonist/antagonist; MC3R agonist; MC4R agonist; NPY1 antagonist; NPY2 agonist; NPY4 agonist; NPY5 antagonist; leptin; leptin agonist/modulator; leptin derivatives; opioid antagonist; orexin antagonist; BRS3 agonist; 11β HSD-1 inhibitor, CCK-A agonist; CNTF; CNTF agonist/modulator; CNTF derivative; DP-IV inhibitor; GHS agonist; UCP-1, 2, and 3 activator; β3 agonist; thyroid hormone β agonist; FAS inhibitor; DGAT1 inhibitor; DGAT2 inhibitor; ACC2 inhibitor; glucocorticoid antagonist; acyl-estrogens; lipase inhibitor; fatty acid transporter inhibitor; dicarboxylate transporter inhibitor; glucose transporter inhibitor; GLP-1 agonist; axokine; metformin; nalmefene; phytopharm compound 57; topiramate; and zonisamide; and pharmaceutically acceptable salts and esters thereof.

In another embodiment of the present invention, the anti-obesity agent is selected from a Mc4r agonist, and pharmaceutically acceptable salts and esters thereof. The melanocortin 4 receptor agonist useful in the compositions of the present invention may be any melanocortin 4 receptor agonist known in the art. The melanocortin 4 receptor agonist may be peptidal or non-peptidal in nature; however, the use of a non-peptidal agent is preferred. Furthermore, the use of a selective melanocortin 4 receptor agonist is preferred. For convenience, the use of an orally active appetite suppressant is also preferred. Melanocortin 4 receptor agonists useful in the methods and medicaments of the present invention are represented by the compounds of structural Formula I and II:

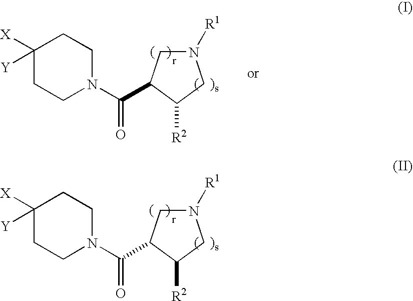

or a pharmaceutically acceptable salt thereof; wherein
X is selected from the group consisting of: C₁₋₈ alkyl, (CH₂)ₙC₃₋₈ cycloalkyl, (CH₂)ₙ-phenyl, (CH₂)ₙ-naphthyl, (CH₂)ₙ-heteroaryl, (CH₂)ₙheterocyclyl, (CH₂)ₙC≡N, (CH₂)ₙCON(R⁵R⁵), (CH₂)ₙCO₂R⁵, (CH₂)ₙCOR⁵, (CH₂)ₙNR⁵C(O)R⁵, (CH₂)ₙNR⁵CO₂R⁵, (CH₂)ₙNR⁵C(O)N(R⁵)₂, (CH₂)ₙNR⁵SO₂R⁵, (CH₂)ₙS(O)ₚR⁵, (CH₂)ₙSO₂N(R⁵)(R⁵), (CH₂)ₙOR⁵, (CH₂)ₙOC(O)R⁵, (CH₂)ₙOC(O)OR⁵, (CH₂)ₙOC(O)N(R⁵)₂, (CH₂)ₙN(R⁵)(R⁵), and (CH₂)ₙNR⁵SO₂N(R⁵)(R⁵); phenyl, naphthyl, and heteroaryl are unsubstituted or substituted with one to three groups independently selected from R⁶; alkyl, cycloalkyl, and heterocyclyl are unsubstituted or substituted with one to three groups independently selected from R⁶ and oxo; and wherein any methylene (CH₂) in X is unsubstituted or substituted with one to two groups independently selected from halogen, hydroxy, and C₁₋₄ alkyl;
Y is selected from the group consisting of: hydrogen, C₁₋₈ alkyl, C₂₋₆ alkenyl, (CH₂)ₙC₃₋₈ cycloalkyl, (CH₂)ₙ-phenyl, (CH₂)ₙ-naphthyl, (CH₂)ₙ-heteroaryl, and (CH₂)ₙ-heterocyclyl; and phenyl, naphthyl, and heteroaryl are unsubstituted or substituted with one to three groups independently selected from R⁶; alkyl, cycloalkyl, and heterocyclyl are optionally substituted with one to three groups independently selected from R⁶ and oxo; and wherein any methylene (CH₂) in Y is unsubstituted or substituted with one to two groups independently selected from halogen, hydroxy, and C₁₋₄ alkyl;
R¹ is selected from the group consisting of: hydrogen, amidino, C₁₋₄ alkyliminoyl, C₁₋₁₀ alkyl, (CH₂)ₙ—C₃₋₇ cycloalkyl, (CH₂)ₙ-phenyl, (CH₂)ₙ-naphthyl, and (CH₂)ₙ-heteroaryl, wherein heteroaryl is selected from the group consisting of (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³; and alkyl and cycloalkyl are unsubstituted or substituted with one to three groups independently selected from R³ and oxo;
R² is selected from the group consisting of: phenyl, naphthyl, and heteroaryl, wherein heteroaryl is selected from the group consisting of: (1) pyridinyl, (2) furyl, (3)thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³;
each R³ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R³ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
each R⁴ is independently selected from the group consisting of: hydrogen, C₁₋₆ alkyl, (CH₂)ₙ phenyl, (CH₂)ₙ-heteroaryl, (CH₂)ₙ-naphthyl, (CH₂)ₙ-heterocyclyl, (CH₂)ₙC₃₋₇ cycloalkyl, and (CH₂)ₙC₃₋₇ bicycloalkyl, wherein alkyl, phenyl, heteroaryl, heterocyclyl, and cycloalkyl are unsubstituted or substituted with one to three groups independently selected from halogen, C₁₋₄ alkyl, hydroxy, and C₁₋₄ alkoxy; or two R⁴ groups together with the atom to which they are attached form a 4- to 8-membered mono- or bicyclic ring system optionally containing an additional heteroatom selected from O, S, and NC₁₋₄ alkyl;
R⁵ is independently selected from the group consisting of: hydrogen, C₁₋₈ alkyl, (CH₂)ₙ-phenyl, (CH₂)ₙ-naphthyl, (CH₂)ₙ-heteroaryl, and (CH₂)ₙC₃₋₇ cycloalkyl; phenyl, naphthyl, and heteroaryl are unsubstituted or substituted with one to three groups independently selected from R³; alkyl and cycloalkyl are unsubstituted or substituted with one to three groups independently selected from R³ and oxo; and wherein any methylene (CH₂) in R⁵ is unsubstituted or substituted with one to two groups independently selected from halogen, hydroxy, and C₁₋₄ alkyl; or two R⁵ groups together with the atom to which they are attached form a 5- to 8-membered mono- or bicyclic ring system optionally containing an additional heteroatom selected from O, S, and NC₁₋₄ alkyl;
each R⁶ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R⁶ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
r is 1 or 2;
s is 0, 1, or 2;
n is 0, 1 or 2; and
p is 0, 1, or 2.

In one embodiment of the compounds of structural formula I and II, R¹ is selected from the group consisting of hydrogen, C₁₋₆ alkyl, (CH₂)₀₋₁C₃₋₆ cycloalkyl, and (CH₂)₀₋₁-phenyl; wherein phenyl is unsubstituted or substituted with one to three groups independently selected from R³; and alkyl and cycloalkyl are optionally substituted with one to three groups independently selected from R³ and oxo. In a class of this embodiment, R¹ is tert-butyl.

In a second embodiment of the compounds of structural formula I and II, R² is phenyl or thienyl optionally substituted with one to three groups independently selected from R³. In a class of this embodiment, R² is phenyl optionally substituted with one to three groups independently selected from R³. In another class of this embodiment, R² is phenyl substituted with one to three groups independently selected from R³. In a subclass of this class, R² is phenyl substituted with two groups independently selected from R³. In a subclass of this subclass, R² is phenyl substituted with two halogen groups.

In a third embodiment of the compounds of structural formula I and II, X is selected from the group consisting of: (CH₂)ₙ-phenyl, (CH₂)ₙ-naphthyl, (CH₂)ₙ-heteroaryl, (CH₂)ₙC₃₋₈ cycloalkyl, and (CH₂)ₙ-heterocyclyl; and phenyl, naphthyl, and heteroaryl are optionally substituted with one to three groups independently selected from R⁶; cycloalkyl and heterocyclyl are optionally substituted with one to three groups independently selected from R⁶ and oxo; and wherein any methylene (CH₂) group in X is unsubstituted or substituted with one to two groups independently selected from halogen, hydroxy, and C₁₋₄ alkyl. In a class of this embodiment, X is selected from the group consisting of (CH₂)₀₋₁-phenyl, (CH₂)₀₋₁-heteroaryl, (CH₂)₀₋₁-heterocyclyl; wherein phenyl and heteroaryl are optionally substituted with one to three groups independently selected from R⁶; heterocyclyl is optionally substituted with one to three groups independently selected from R⁶ and oxo; and CH₂ is unsubstituted or substituted with one to two groups independently selected from halogen, hydroxy, and C₁₋₄ alkyl. In a subclass of this class, X is phenyl optionally substituted with one to three groups independently selected from R⁶.

In a fourth embodiment of compounds of formula I and II, Y is hydrogen.

In yet a further embodiment of compounds of structural formula I and II, r is 1 or 2 and s is 1.

In a class of this embodiment, the anti-obesity agent is a Mc4r agonist selected from the group consisting of:
(1) 2-[2-(1-{[(3S,4R)-1-tert-butyl-4-(2,4-difluorophenyl)pyrrolidin-3-yl]carbonyl}piperidin-4-yl)-5-chloro phenyl]-N-methylcarboxamide,
(2) 2-[2-(1-{[(3S,4R)-1-tert-butyl-4-(2,4-difluorophenyl)pyrrolidin-3-yl]carbonyl}piperidin-4-yl)-5-fluoro-phenyl]-N-methylcarboxamide,
(3) 2-[2-(1-{[(3S,4R)-1-tert-butyl-4-(2,4-difluorophenyl)pyrrolidin-3-yl]carbonyl}piperidin-4-yl)-5-methyl-phenyl]-N-methylcarboxamide,
(4) 2-[2-(1-{[(3S,4R)-1-tert-butyl-4-(2,4-difluorophenyl)pyrrolidin-3-yl]carbonyl}piperidin-4-yl)-phenyl]-N-methylcarboxamide,
(5) 2-[2-(1-{[(3S,4R)-1-tert-butyl-4-(2,4-difluorophenyl)pyrrolidin-3-yl]carbonyl}piperidin-4-yl)-4-methyl-phenyl]-N-methylcarboxamide,
(6) 2-[2-(1-{[(3S,4R)-1-tert-butyl-4-(2,4-difluorophenyl)pyrrolidin-3-yl]carbonyl}piperidin-4-yl)-4-fluoro-phenyl]-N-methylcarboxamide,
(7) 4-[2-(2-azetidin-1-yl-1(S)-methyl-2-oxoethyl)-4-chlorophenyl]-1-{[(3S,4R)-1-tert-butyl-4-(2,4-difluorophenyl)pyrrolidin-3-yl]carbonyl}piperidine,
(8) 4-[2-(2-azetidin-1-yl-1(R)-methyl-2-oxoethyl)-4-chlorophenyl]-1-{[(3S,4R)-1-tert-butyl-4-(2,4-difluorophenyl)pyrrolidin-3-yl]carbonyl}piperidine,
(9) 4-[2-(2-azetidin-1-yl-1,1-dimethyl-2-oxoethyl)-4-chlorophenyl]-1-{[(3S,4R)-1-tert-butyl-4-(2,4-difluorophenyl)pyrrolidin-3-yl]carbonyl}piperidine,
(10) 4-[2-(2-azetidin-1-yl-1-cyclopropyl-2-oxoethyl)-4-chlorophenyl]-1-{[(3S,4R)-1-tert-butyl-4-(2,4-difluorophenyl)pyrrolidin-3-yl]carbonyl}piperidine,
(11) 4-[2-(2-azetidin-1-yl-1,1-dimethyl-2-oxoethyl)-4-fluorophenyl]-1-{[(3S,4R)-1-tert-butyl-4-(2,4-difluorophenyl)pyrrolidin-3-yl]carbonyl}piperidine,
(12) 4-[2-(2-azetidin-1-yl-1-cyclopropyl-2-oxoethyl)-4-fluorophenyl]-1-{[(3S,4R)-1-tert-butyl-4-(2,4-difluorophenyl)pyrrolidin-3-yl]carbonyl}piperidine,
(13) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide,
(14) N-{(1R)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide,
(15) N-{1-[2-(1-{[(3S,4R)-1-tert-butyl-4-(2,4-difluorophenyl)pyrrolidin-3-yl]carbonyl}piperidin-4-yl)-5-chlorophenyl]-1-methylethyl}acetamide,
(16) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}acetamide,
(17) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}cyclobutanecarboxamide,
(18) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}propanamide,
(19) N-{1-[2-(1-{[(3S,4R)-1-tert-butyl-4-(2,4-difluoro-phenyl)pyrrolidin-3-yl]carbonyl}piperidin-4-yl)-5-chlorophenyl]ethyl}-N-methylurea,
(20) Methyl-2-[2-(1-{[(3S,4R)-1-tert-butyl-4-(2,4-difluoro-phenyl)pyrrolidin-3-yl]carbonyl}piperidin-4-yl)-5-chlorophenyl]-2-methylpropanoate,
(21) N-{1-[2-(1-{[(3S,4R)-1-tert-butyl-4-(2,4-difluoro-phenyl)pyrrolidin-3-yl]carbonyl}piperidin-4-yl)-5-fluorophenyl]-1-methylethyl}acetamide,
(22) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}-N-methylurea,
(23) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}cyclobutanecarboxamide,
(24) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}propanamide,
(25) N-{(1S)-1-[2-(1-{[(3S,4R)-1-tert-butyl-4-(2,4-difluoro-phenyl)pyrrolidin-3-yl]carbonyl}piperidin-4-yl)-5-fluorophenyl]ethyl}acetamide,
(26) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]propyl}acetamide, and
(27) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}pyrimidine-5-carboxamide,
   and pharmaceutically acceptable salts thereof.

In a sub-class of this class, the anti-obesity agent is a Mc4r agonist selected from the group consisting of:
(1) N-{(1S)-1-[2-(1-{[(3S,4R)-1-tert-butyl-4-(2,4-difluoro-phenyl)pyrrolidin-3-yl]carbonyl}piperidin-4-yl)-5-fluorophenyl]ethyl}acetamide,
(2) N-{(1S)-1-[2-(1-{[(3S,4R)-1-tert-butyl-4-(2,4-difluoro-phenyl)pyrrolidin-3-yl]carbonyl}piperidin-4-yl)-5-chlorophenyl]propyl}acetamide,
(3) N-{(1S)-1-[2-(1-{[(3S,4R)-1-tert-butyl-4-(2,4-difluoro-phenyl)pyrrolidin-3-yl]carbonyl}piperidin-4-yl)-5-chlorophenyl]ethyl}acetamide,
(4) 2-[2-(1-{[(3S,4R)-1-tert-butyl-4-(2,4-difluorophenyl)pyrrolidin-3-yl]carbonyl}piperidin-4-yl)-5-chloro phenyl]-N-methylcarboxamide,
(5) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}pyrimidine-5-carboxamide, and
(6) 4-[2-(2-azetidin-1-yl-1(S)-methyl-2-oxoethyl)-4-chlorophenyl]-1-{[(3S,4R)-1-tert-butyl-4-(2,4-difluorophenyl)pyrrolidin-3-yl]carbonyl}piperidine,
   and pharmaceutically acceptable salts thereof.

In another class of this embodiment, the Mc4r agonist is compound A. Compound A has the following structure:

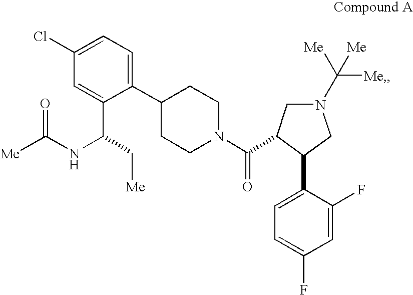

and pharmaceutically acceptable salts, esters and tautomers thereof. Compound A, and its preparation are disclosed in WO 02/068388. Compound A is a selective agonist for MC4R, with a selective functional activity characterized by an EC₅₀ value for the melanocortin 4 receptor that is at least 120-fold lower than for the melanocortin 1 receptor, at least 700-fold lower than for the melanocortin 2 receptor, at least 65-fold lower than for the melanocortin 3 receptor, and at least 100 fold lower than for the melanocortin 5 receptor.

Other melanocortin-4 receptor agonists useful in the methods of the present invention include, but are not limited to, the following:

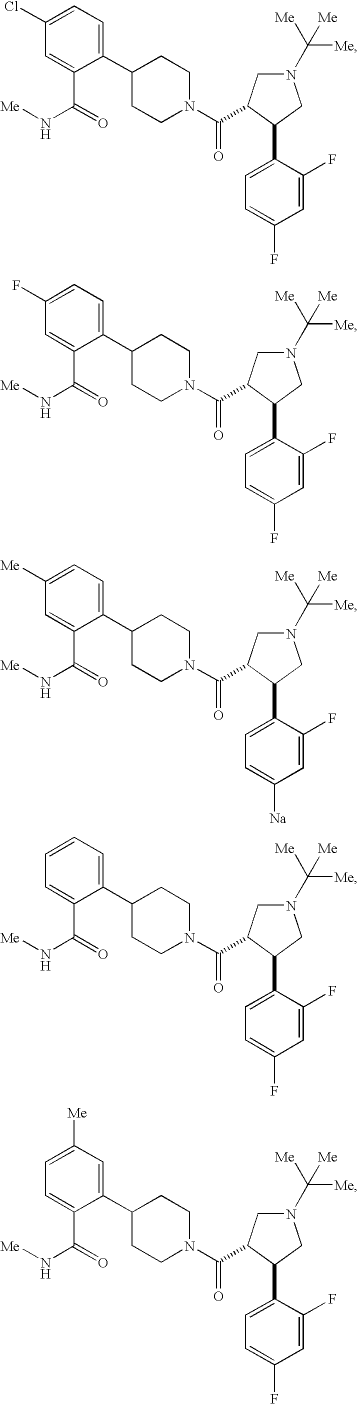

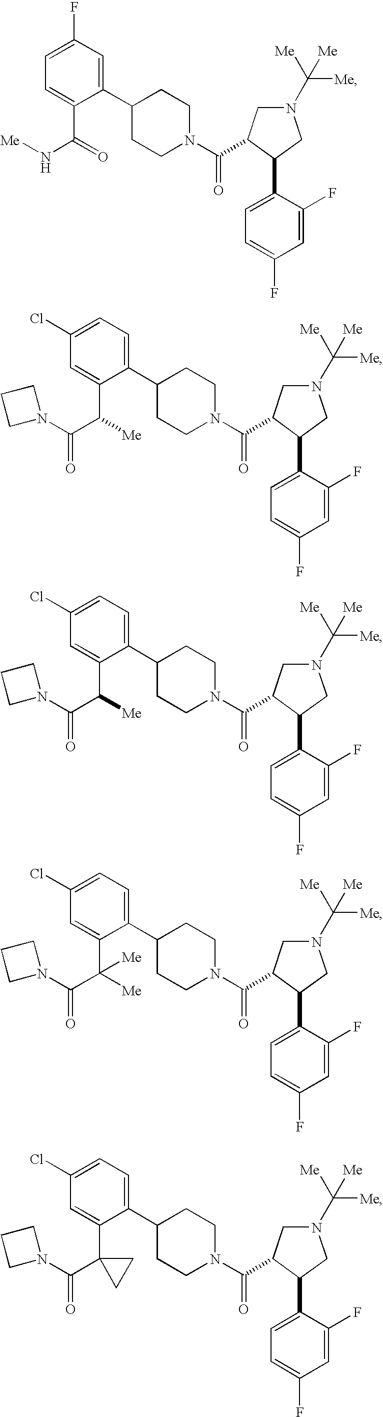

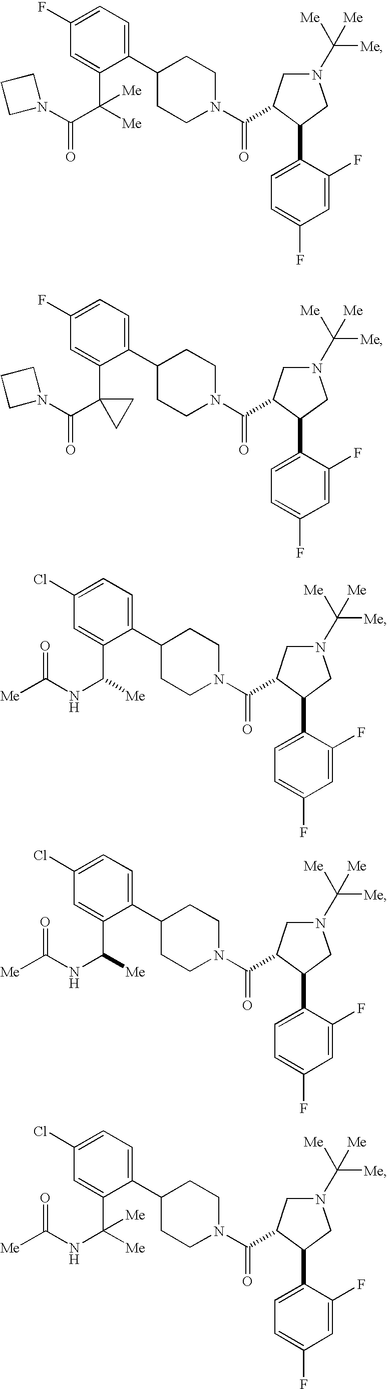

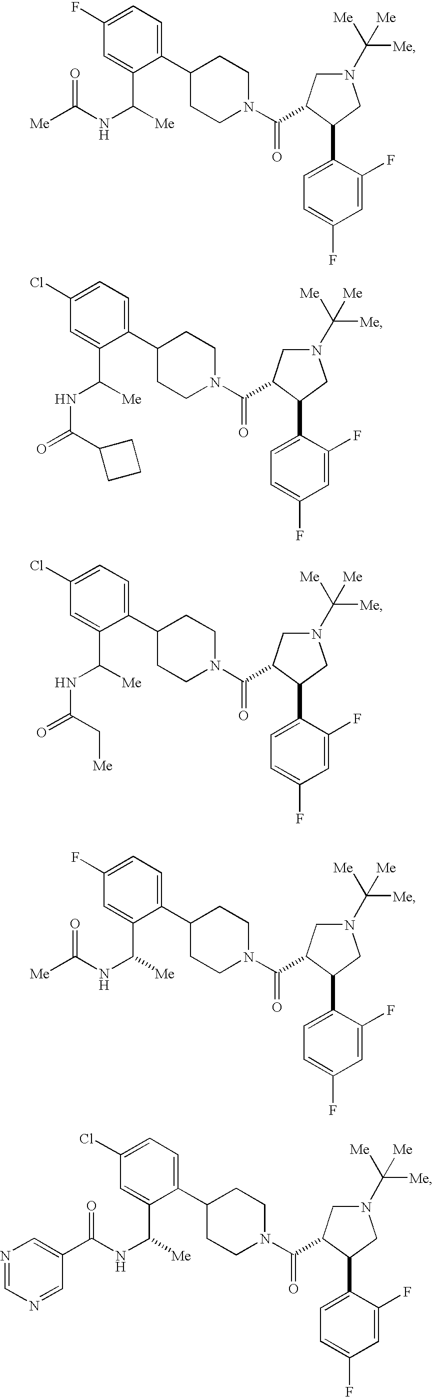

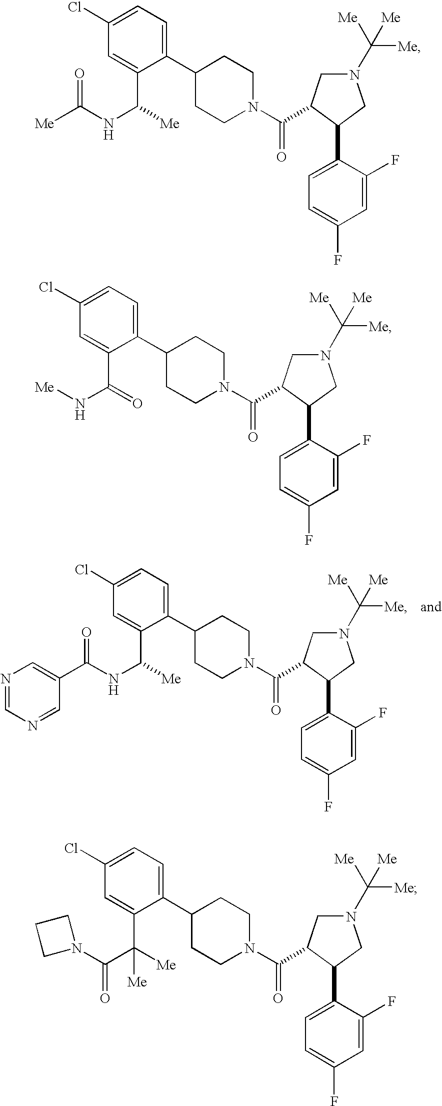

or a pharmaceutically acceptable salt thereof.

The melanocortin 4 receptor agonists of Formula I and II, including Compound A, and their preparation are disclosed in US2003/0225060, which is hereby incorporated by reference in its entirety, and in WO 02/068388.

Other melanocortin 4 receptor agonists useful in the present invention, include but are not limited to those disclosed in U.S. Pat. No. 6,294,534, U.S. Pat. Nos. 6,350,760, 6,376,509, 6,410,548, 6,458,790, U.S. Pat. No. 6,472,398, U.S. Pat. No. 5,837,521, U.S. Pat. No. 6,699,873, which are hereby incorporated by reference in their entirety; in US Patent Application Publication Nos. US 2002/0004512, US2002/0019523, US2002/0137664, US2003/0236262, US2003/0225060, US2003/0092732, US2003/109556, US 2002/0177151, US 2002/187932, US 2003/0113263, which are hereby incorporated by reference in their entirety; and in WO 99/64002, WO 00/74679, WO 02/15909, WO 01/70708, WO 01/70337, WO 01/91752, WO 02/068387, WO 02/068388, WO 02/067869, WO 03/007949, WO 2004/024720, WO 2004/037797, WO 01/58891, WO 02/070511, WO 02/079146, WO 03/009847, WO 03/057671, WO 03/068738, WO 03/092690, WO 02/059095, WO 02/059107, WO 02/059108, WO 02/059117, WO 02/085925, WO 03/004480, WO 03/009850, WO 03/013571, WO 03/031410, WO 03/053927, WO 03/061660, WO 03/066597, WO 03/094918, WO 03/099818, WO 04/037797, WO 04/048345, WO 04/058735, WO 02/018327, WO 02/080896, WO 02/081443, WO 03/066587, WO 03/066597, WO 03/099818, WO 02/062766, WO 03/000663, WO 03/000666, WO 03/003977, WO 03/040107, WO 03/040117, WO 03/040118, WO 03/013509, WO 03/057671, WO 02/079753, WO 02/092566, WO 03/093234, WO 03/095474, WO 03/104761, WO 2004/078716, and 2004/078717. Specific Mc4r agonists useful in the present invention include CHIR 86036 (Chiron); ME-10142; ME-10145 (Melacure); MT-II, PT-141, and PT-14 (Palatin).

One of ordinary skill in the art can readily identify melanocortin 4 receptor agonist compounds useful in the compositions and methods of the present invention using the methods described in Example 1. MC4R agonists which are useful in the present invention generally have an IC₅₀ less than 100 nM in the MC4R agonist binding assay and an EC₅₀ less than 100 nM in the functional assay described in Example 5. Particularly useful in the present invention are MC4R agonists with an IC₅₀ less than 45 nM or an EC₅₀ less than 15 nM. More particularly useful in the present in invention are MC4R agonists with an EC₅₀ less than 15 nM and an IC₅₀ less than 45 nM.

The methods and compositions of the present invention comprise a neurokinin-1 antagonist. The neurokinin-1 antagonist useful in the compositions of the present invention may be any neurokinin-1 antagonist known in the art. The neurokinin-1 antagonist may be peptidal or non-peptidal in nature; however, the use of a non-peptidal agent is preferred. Furthermore, the use of a selective neurokinin-1 antagonist is preferred. Additionally, the use of a central nervous system (CNS) penetrant neurokinin-1 antagonist is preferred. For convenience, the use of an orally active neurokinin-1 antagonist is also preferred.

Neurokinin-1 receptor antagonists of use in the present invention are fully described, for example, in U.S. Pat. Nos. 5,162,339, 5,232,929, 5,242,930, 5,373,003, 5,387,595, 5,459,270, 5,494,926, 5,496,833, 5,637,699; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942, 97/21702, and 97/49710; and in British Patent Publication Nos. 2 266 529, 2 268 931, 2 269 170, 2 269 590, 2 271 774, 2 292 144, 2 293 168, 2 293 169, and 2 302 689.

Specific Neurokinin-1 antagonist useful in the methods and medicaments of the present invention include:
(1) (±)-(2R3R,2S3S)—N-{[2-cyclopropoxy-5-(trifluoromethoxy)-phenyl]methyl}-2-phenylpiperidin-3-amine;
(2) 2-(S)-(3,5-bis(trifluoromethyl)benzyloxy)-3(S)-(4-fluorophenyl)-4-(3-(5-oxo-1H,4H-1,2,4-triazolo)methyl)morpholine;
(3) 2-(R)-(1-(R)-(3,5-bis(trifluoromethyl)phenyl)ethoxy)-4-(3-(5-oxo-1H,4H-1,2,4-triazolo)methyl)-3-(S)-phenyl-morpholine;
(4) 2-(S)-(3,5-bis(trifluoromethyl)benzyloxy)-4-(3-(5-oxo-1H,4H-1,2,4-triazolo)methyl)-3-(S)-phenyl-morpholine;
(5) 2-(R)-(1-(R)-(3,5-bis(trifluoromethyl)phenyl)ethoxy)-3-(S)-(4-fluorophenyl)-(3-(5-oxo-1H,4H-1,2,4-triazolo)methyl)morpholine;
(6) 2-(R)-(1-(R)-(3,5-bis(trifluoromethyl)phenyl)ethoxy)-4-(5-(N,N-dimethylamino)methyl-1,2,3-triazol-4-yl)methyl-3-(S)-phenylmorpholine;
(7) 2-(R)-(1-(R)-(3,5-bis(trifluoromethyl)phenyl)ethoxy)-4-(5-(N,N-dimethylamino)methyl-1,2,3-triazol-4-yl)methyl-3-(S)-(4-fluorophenyl)morpholine;
(8) (3S,5R,6S)-3-[2-cyclopropoxy-5-(trifluoromethoxy)phenyl]-6-phenyl-1-oxa-7-aza-spiro[4.5]decane;
(9) (3R,5R,6S)-3-[2-cyclopropoxy-5-(trifluoromethoxy)phenyl]-6-phenyl-1-oxa-7-aza-spiro[4.5]decane;
(10) 2-(R)-(1-(S)-(3,5-bis(trifluoromethyl)phenyl)-2-hydroxyethoxy)-3-(S)-(4-fluorophenyl)-4-(1,2,4-triazol-3-yl)methylmorpholine;
(11) 2-(R)-(1-(R)-(3,5-bis(trifluoromethyl)phenyl)ethoxy)-3-(S)-(4-fluorophenyl)-4-(3-(4-monophosphoryl-5-oxo-1H-1,2,4-triazolo)methyl)morpholine;
(12) 2-(R)-(1-(R)-(3,5-bis(trifluoromethyl)phenyl)ethoxy)-3-(S)-(4-fluorophenyl)-4-(3-(1-monophosphoryl-5-oxo-1H-1,2,4-triazolo)methyl)morpholine;
(13) 2-(R)-(1-(R)-(3,5-bis(trifluoromethyl)phenyl)ethoxy)-3-(S)-(4-fluorophenyl)-4-(3-(2-monophosphoryl-5-oxo-1H-1,2,4-triazolo)methyl)morpholine;
(14) 2-(R)-(1-(R)-(3,5-bis(trifluoromethyl)phenyl)ethoxy)-3-(S)-(4-fluorophenyl)-4-(3-(5-oxyphosphoryl-1H-1,2,4-triazolo)methyl)morpholine;
(15) 2-(S)-(1-(R)-(3,5-bis(trifluoromethyl)phenyl)ethoxy)-3-(S)-(4-fluorophenyl)-4-(3-(1-monophosphoryl-5-oxo-4H-1,2,4-triazolo)methyl)morpholine;
(16) 2-(R)-(1-(R)-(3,5-bis(trifluoromethyl)phenyl)ethoxy)-4-(4-N,N-dimethylaminobut-2-yn-yl)-3-(S)-(4-fluorophenyl)morpholine;
   or a pharmaceutically acceptable salt thereof.

The preparation of these compounds is fully described in the aforementioned patents and publications.

Another specific neurokinin-1 antagonist useful in the methods and medicaments of the present invention is Compound B. Compound B is 2-(R)-(1-(R)-(3,5-bis(trifluoromethyl)phenyl)ethoxy)-4-(5-(dimethylamino)methyl-1,2,3-triazol-4-yl)methyl-3-(S)-(4-fluorophenyl)morpholine, and pharmaceutically acceptable salts, esters and tautomers thereof. Compound B and its preparation are disclosed in U.S. Pat. No. 6,432,952, which is hereby incorporated herein in its entirety.

One of ordinary skill in the art can readily identify neurokinin-1 antagonists useful in the compositions and methods of the present invention using the binding assays and functional assays described in Examples 14. Neurokinin I antagonists which are useful in the present invention generally have an IC₅₀ less than 1 μM in the Neurokinin-1 receptor binding assay described in Example 1. Neurokinin-1 antagonists which are preferred in the present invention generally have an IC₅₀ less than 100 nM in the NK-1 Receptor binding assay (See Example 1), more preferably an IC₅₀ of less than 10 nM, most preferably an IC₅₀ less than 1 nM.

The class of orally active, long acting, CNS-penetrant NK-1 receptor antagonists of use in the present invention is identified using a combination of 1) the NK-1 Receptor binding assay; 2) the Gerbil Foot-Tapping assay; and 3) the Ferret Emesis assay; and 4) the Separation-Induced Vocalisation assay. A suitable selection cascade for NK-1 receptorantagonists of use according to the present invention is as follows: (i) Determine affinity for human NK-1 receptor in radioligand binding studies (Assay 1); select compounds with IC₅₀≦10 nM, preferably IC₅₀≦2 nM, especially IC₅₀≦1 nM. (ii) Determine ability of compounds to penetrate CNS by their ability to inhibit foot tapping in gerbils induced by central injection of an NK-1 agonist (Assay 2); select compounds that inhibit foot tapping with ID₅₀≦3 mg/kg i.v., and preferably ID₅₀≦1 mg/kg i.v. when administered immediately prior to central NK-1 agonist challenge, or ID₅₀≦30 mg/kg p.o., and preferably ID₅₀≦10 mg/kg p.o. 1 hour prior to challenge. (iii) Determine central duration of action of compounds in gerbil foot tapping assay following intravenous administration 24 hours prior to central NK-1 agonist challenge; select compounds showing ≦25-fold loss of potency compared with ID₅₀ determined in step (ii) above with the proviso that ID₅₀≦10 mg/kg i.v., and preferably ≦5 mg/kg i.v. after 24 hour pre-treatment. (iv) Determine oral bioavailability of compounds by pharmacokinetic analysis, activity in gerbil foot tapping assay following oral administration and/or by ability to inhibit cisplatin-induced emesis in ferrets (Assay 3); select compounds with ID₉₀≦3 mg/kg p.o., and preferably ID₅₀≦1 mg/kg p.o.

Particularly preferred compounds of use in the present invention are identified using steps (i) to (iv) followed by step (v): (v) Determine activity of compounds in assays sensitive to conventional antidepressant/anxiolytic drugs (inhibition of pharmacologically evoked foot tapping in gerbils and/or inhibition of distress vocalisations in guinea-pig pups (Assay 4)). Select compounds with ID₅₀≦20 mg/kg, and preferably ID₅₀≦10 mg/kg. Yet further preferred compounds of use in the present invention may be selected from those compounds which satisfy the NK-1 receptor binding criteria of step (i) which, in addition, have ≦5-fold shift in affinity when incubated in the presence of human serum albumin (HSA) to show non-specific protein binding. One example of a NK-1 receptor antagonist of use in the present invention is the compound 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine, the preparation of which is described in International Patent Specification No. WO 95/16679. In the aforementioned assays, this compound has the following activity: human NK-1 receptor binding: IC₅₀=0.1 nM; gerbil foot-tapping (5 mins.): ID₅₀=0.36 mg/kg i.v.; gerbil foot-tapping (24 hrs.): ID₅₀=0.33 mg/kg i.v.; ferret emesis: ID₉₀<3 mg/kg p.o. guinea-pig vocalisation; and (4 hr. pre-treatment): ID₅₀=0.73 mg/kg p.o.

As used herein, the term “anti-obesity agent” includes compounds that reduce total food intake by 5 to 30%, or reduce caloric intake or selectively reduce intake of specific components of the diet such as carbohydrates or fats by 5 to 30%; compounds which, when administered to a subject, act to increase the metabolic rate of the subject, particularly those agents which increase metabolic rate by at least 5%, preferably 10%, most preferably 20% in 24 hour energy expenditure when administered to the subject; and compounds that inhibit the absorption of 10 to 50% of the nutrients.

One of ordinary skill in the art can readily identify anti-obesity agents useful in the compositions and methods of the present invention. Anti-obesity agents that decrease food intake can be evaluated in rodents according to the procedures described in: Daniels, A. J. et al., Regulatory Peptides, 106:47-54 (2002); Halaas, J. L. et. al., Science, 269: 543-546 (1995); and Strack, A. M., Obesity Research, 10: 173-81 (2002). Anti-obesity agents that increase metabolic rate are routinely evaluated in rodents (Atgie, C., Comp. Biochem. Physiol. A. Mol. Integr. Physiol. 119:629-36 (1998); Himms-Hagan, J., American J. Physiology, 266:R1371-82 (1994)), and, even when inactive in rodents, are tested in additional species such as dog and monkey before ultimately being tested in humans (Connacher, A. A. et. al., Int'l J. Obesity, 16: 685-694 (1992); Connacher, A. A. et. al., Am. J. Clin. Nutr., 55: 258S-261S (1992); Connacher, A. A. et. al., Brit. Med. J., 296: 1217-1220 (1998)). The utility of anti-obesity agents that enhance metabolic rate is supported by experiments with mice, in which the RII-beta gene has been deleted, that were shown to be resistant to diet induced obesity (D. E. Cummings et al. Nature 382: 622-626 (1996)). Anti-obesity agents that inhibit nutrient absorption can be evaluated in: Badr M. Z. and Chen, T. S., Toxicology, 34:333-40 (1985); Sorribas, V., J. Pharm. Pharmacol., 44:1030-2 (1992).

Serotonin (5HT) transport inhibitors useful in this invention include, but are not limited to, paroxetine, fluoxetine, fenfluramine, fluvoxamine, sertraline, and imipramine.

Norepinephrine (NE) transport inhibitors useful in this invention include, but are not limited to, GW 320659, despiramine, talsupram, and nomifensine.

Cannabinoid receptor 1 (CB-1) antagonists/inverse agonists useful in the present invention include: U.S. Pat. Nos. 5,532,237, 4,973,587, 5,013,837, 5,081,122, 5,112,820, 5,292,736, 5,624,941 and U.S. Pat. No. 6,028,084; US Patent Publication No. US 2004/048317, which are incorporated by reference herein; and PCT Application Nos. WO 96/33159, WO 98/33765, WO98/43636, WO98/43635, WO 01/09120, WO 01.96330, WO98/31227, WO98/41519, WO98/37061, WO00/10967, WO00/10968, WO97/29079, WO99/02499, WO 01/58869, WO 02/076949, WO 01/64632, WO 01/64633, WO 01/64634, WO 03/006007, WO 03/077847; WO 03/007887, and WO 2004/048317; and EPO Application No. EP-658546. Specific CB-1 antagonists/inverse agonists useful in the present invention include, but are not limited to, rimonabant (Sanofi Synthelabo), SR-147778 (Sanofi Synthelabo), BAY 65-2520 (Bayer), and SLV 319 (Solvay).

Ghrelin antagonists useful in the present invention, include, but are not limited to, those disclosed in: PCT Application Nos. WO 01/87335, and WO 02/08250.

Histamine 3 (H3) antagonist/inverse agonists useful in the present invention include, but are not limited to, those disclosed in: PCT Application No. WO 02/15905; and O-[3-(1H-imidazol-4-yl)propanol]carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm. (Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)). Specific H3 antagonists/inverse agonists useful in the present invention include, but are not limited to, thioperamide, 3-(1H-imidazol-4-yl)propyl N-(4-pentenyl)carbamate, clobenpropit, iodophenpropit, imoproxifan, and GT2394 (Gliatech).

Melanin-concentrating hormone 1 receptor (MCH1R) antagonists useful in the present invention include, but are not limited to, those disclosed in: Melanin-concentrating hormone 1 receptor (MCH1R) antagonists, such as T-226296 (Takeda), SNP-7941 (Synaptic), and the compounds disclosed in PCT Patent Application Nos. WO 01/82925, WO 01/87834, WO 02/051809, WO 02/06245, WO 02/076929, WO 02/076947, WO 02/04433, WO 02/51809, WO 02/083134, WO 02/094799, WO 03/004027, and Japanese Patent Application Nos. JP 13226269, and JP 2004-139909.

Neuropeptide Y1 (NPY1) antagonists useful in the present invention, include, but are not limited to, those disclosed in: U.S. Pat. No. 6,001,836; and PCT Application Nos. WO 96/14307, WO 01/23387, WO 99/51600, WO 01/85690, WO 01/85098, WO 01/85173, and WO 01/89528. Specific examples of NPY1 antagonists useful in the present invention include, but are not limited to, BIBP3226, J-115814, BIBO 3304, LY-357897, CP-671906, and GI-264879A.

Neuropeptide Y2 (NPY2) agonists useful in the present invention, include, but are not limited to, PYY, PYY 3-36, and the compounds disclosed in U.S. Pat. No. 5,026,685, U.S. Pat. No. 5,604,203, U.S. Pat. No. 5,574,010, U.S. Pat. No. 5,696,093, U.S. Pat. No. 5,936,092, U.S. Pat. No. 6,046,162, U.S. Pat. No. 6,046,167, U.S. Pat. No. 6,093,692, U.S. Pat. No. 6,225,445, U.S. Pat. No. 4,002,531, U.S. Pat. No. 4,179,337, U.S. Pat. No. 5,122,614, U.S. Pat. No. 5,349,052, U.S. Pat. No. 5,552,520, U.S. Pat. No. 5,604,203, U.S. Pat. No. 6,127,355, WO 95/06058, WO 98/32466, WO 03/026591, and WO 2004/066966, which are incorporated herein by reference; Olitvak, D. A. et al., Dig. Dis. Sci. 44(3):64348 (1999), Liu, C D. et al., Surg. Res. 59(1):80-84 (1995); Batterham, et al., Nature. 418:650-654 (2003), NPY3-36, and other Y2 agonists such as N acetyl [Leu(28,31)] NPY 24-36 (White-Smith and Potter, Neuropeptides 33:526-33 (1999)), TASP-V (Malis et al., Br. J. Pharmacol. 126:989-96 (1999)), cyclo-(28/32)-Ac-[Lys28-Glu32]-(25-36)-pNPY (Cabrele and Beck-Sickinger, J-Pept-Sci. 6:97-122 (2000)), BIM43073D, and BIM-43004C.

Neuropeptide Y4 (NPY4) agonists useful in the present invention, include, but are not limited to, compounds such as pancreatic peptide (PP) as described in Batterham et al., J. Clin. Endocrinol. Metab. 88:3989-3992 (2003), and other Y4 agonists such as 1229U91 (Raposinho et al., Neuroendocrinology. 71:2-7 (2000)).

NPY5 antagonists useful in the present invention, include, but are not limited to, the compounds disclosed in: U.S. Pat. Nos. 6,057,335; 6,043,246; 6,140,354; 6,166,038; 6,180,653; 6,191,160; 6,313,298; 6,335,345; 6,337,332; 6,326,375; 6,329,395; 6,340,683; 6,388,077; 6,462,053; 6,649,624; and 6,723,847, hereby incorporated by reference in their entirety; European Patent Nos. EP-01010691, and EP-01044970; and PCT International Patent Publication Nos. WO 97/19682, WO 97/20820, WO 97/20821, WO 97/20822, WO 97/20823, WO 98/24768; WO 98/25907; WO 98/25908; WO 98/27063, WO 98/47505; WO 98/40356; WO 99/15516; WO 99/27965; WO 00/64880, WO 00/68197, WO 00/69849, WO 01/09120, WO 01/14376; WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/22592, WO 0248152, and WO 02/49648; WO 02/094825; WO 03/014083; WO 03/10191; WO 03/092889; WO 04/002986; and WO 04/031175. Specific NPY 5 antagonists useful in the combinations of the present invention, include, but are not limited to GW-569180A, GW-594884A, GW-587081X, GW-548118×; FR226928, FR 240662, FR252384; 1229U91, GI-264879A, CGP71683A, LY-377897, PD-160170, SR-120562A, SR-120819A and JCF-104. Additional specific NPY 5 antagonists useful in the combinations of the present invention, include, but are not limited to the compounds described in Norman et al., J. Med. Chem. 43:4288-4312 (2000).

One of ordinary skill in the art can readily identify other NPY5 antagonist compounds useful in the compositions and methods of the present invention using the methods described in WO 96/16542. NPY5 antagonists which are useful in the present invention generally have an IC₅₀ less than 1 uM in the NPY Y5 binding assay described in Kanatani et al., Biochem. Biophys. Res. Commun. 272:169-173 (2000). NPY5 antagonists which are preferred in the present invention generally have an IC₅₀ less than 100 nM in the NPY Y5 binding assay.

Leptin includes, but is not limited to, recombinant human leptin (PEG-OB, Hoffman La Roche) and recombinant methionyl human leptin (Amgen). Leptin derivatives (e.g., truncated forms of leptin) useful in the present invention include, but are not limited to, those disclosed in: Pat. Nos. 5,552,524; 5,552,523; 5,552,522; 5,521,283; and PCT International Publication Nos. WO 96/23513; WO 96/23514; WO 96/23515; WO 96/23516; WO 96/23517; WO 96/23518; WO 96/23519; and WO 96/23520.

Opioid antagonists useful in the present invention include, but are not limited to, those disclosed in: PCT Application No. WO 00/21509. Specific opioid antagonists useful in the present invention include, but are not limited to, nalmefene (Revex®D), 3-methoxynaltrexone, naloxone, and naltrexone.

Orexin antagonists useful in the present invention include, but are not limited to, those disclosed in: PCT Patent Application Nos. WO 01/96302, WO 01/68609, WO 02/51232, and WO 02/51838. Specific orexin antagonists useful in the present invention include, but are not limited to, SB-334867-A.

An acyl-estrogen useful in the present invention include oleoyl-estrone (del Mar-Grasa, M. et al., Obesity Research, 9:202-9 (2001)).

11β HSD-1 (11-beta hydroxy steroid dehydrogenase type 1) inhibitor useful in the present invention include, but are not limited to, BVT 3498, BVT 2733, and those disclosed in WO 01/90091, WO 01/90090, WO 01/90092, and U.S. Pat. No. 6,730,690 and US Publication No. US 2004-0133011, which are incorporated by reference herein in their entirety.

Cholecystokinin-A (CCK-A) agonists useful in the present invention include, but are not limited to, those disclosed in U.S. Pat. No. 5,739,106. Specific CCK-A agonists include, but are not limited to, AR-R 15849, GI 181771, JMV-180, A-71378, A-71623 and SR146131.

Specific ciliary neurotrophic factors (CNTF) useful in the present invention include, but are not limited to, GI-181771 (Glaxo-SmithKline); SR146131 (Sanofi Synthelabo); butabindide; PD170,292, PD 149164 (Pfizer). CNTF derivatives useful in the present invention include, but are not limited to, axokine (Regeneron); and those disclosed in PCT Application Nos. WO 94/09134, WO 98/22128, and WO 99/43813.

Specific Cox-2 inhibitors useful in the present invention include, but are not limited to, rofecoxib (VIOXX®, see U.S. Pat. No. 5,474,995, hereby incorporated by reference in its entirety), etoricoxib (ARCOXIA™ see U.S. Pat. No. 5,861,419, hereby incorporated by reference in its entirety), celecoxib (CELEBREX®, see U.S. Pat. No. 5,466,823, hereby incorporated by reference in its entirety), valdecoxib (see U.S. Pat. No. 6,633,272, hereby incorporated by reference in its entirety), parecoxib (see U.S. Pat. No. 5,932,598, hereby incorporated by reference in its entirety), lumiracoxib (PREXIGE®, Novartis), BMS347070 (Bristol Myers Squibb), tiracoxib or JTE522 (Japan Tobacco), ABT963 (Abbott), CS502 (Sankyo) and GW406381 (GlaxoSmithKline), and pharmaceutically acceptable salts thereof.

DP-IV Inhibitors useful in the present invention include, but are not limited to, isoleucine thiazolidide, valine pyrrolidide, NVP-DPP728, LAF237, P93/01, TSL 225, TMC-2A/2B/2C, FE 999011, P9310/K364, VIP 0177, SDZ 274-444; and the compounds disclosed in U.S. Pat. No. 6,699,871, which is incorporated herein by reference; and International Patent Application Nos. WO 03/004498; WO 03/004496; EP 1 258 476; WO 02/083128; WO 02/062764; WO 03/000250; WO 03/002530; WO 03/002531; WO 03/002553; WO 03/002593; WO 03/000180; and WO 03/000181.

Growth hormone secretagogue (GHS) agonists useful in the present invention include, but are not limited to, those disclosed in: U.S. Pat. No. 6,358,951, and U.S. Patent Application Nos. 2002/049196 and 2002/022637; and PCT Application Nos. WO 01/56592, and WO 02/32888. Specific GHS agonists include, but are not limited to, NN₇O₃, hexarelin, MK-0677, SM-130686, CP424,391, L-692,429 and L-163,255.

5HT2C agonists useful in the present invention include, but are not limited to, those disclosed in: U.S. Pat. No. 3,914,250; and PCT Application Nos. WO 02/36596, WO 02/48124, WO 02/10169, WO 01/66548, WO 02/44152; WO 02/51844, WO 02/40456, and WO 02/40457. Specific 5HT2C agonists useful in this invention include, but are not limited to, BVT933, DPCA37215, WAY161503, and R-1065.

Monoamine reuptake inhibitors useful in the present invention include, but are not limited to, those disclosed in: PCT Application Nos. WO 01/27068, and WO 01/62341. Specific monoamine reuptake inhibitors useful in the present invention include, but are not limited to, sibutramine (Meridia ®/Reductil®) disclosed in U.S. Pat. Nos. 4,746,680, 4,806,570, and 5,436,272, and U.S. Patent Publication No. 2002/0006964. The present invention encompasses sibutratmine as a racemic mixture, as optically pure isomers (+) and (−), or a pharmaceutically acceptable salt, solvent, hydrate, clathrate or prodrug thereof; particularly sibutramine hydrochloride monohydrate.

Serotonin reuptake inhibitors useful in the present invention include, but are not limited to, those disclosed in: U.S. Pat. No. 6,365,633; and PCT Patent Application Nos. WO 01/27060, and WO 01/162341.

Uncoupling Protein (UCP-1, UCP-2, and UCP-3) activators useful in the present invention include, but are not limited to, those disclosed in: PCT Patent Application No. WO 99/00123. Specific uncoupling protein (UCP-1, UCP-2, and UCP-3) activators useful in the present invention include, but are not limited to, phytanic acid, 4-[(E)-2-(5,6,7,8-tetrahydro-5,5,8,8-tetramethyl-2-napthalenyl)-1-propenyl]benzoic acid (TTNPB), and retinoic acid.

β3 adrenergic receptor (β3) agonists useful in the present invention include, but are not limited to, those disclosed in: U.S. Pat. No. 5,705,515, and U.S. Pat. No. 5,451,677; and PCT Patent Application Nos. WO 01/74782, and WO 02/32897. Specific β3 agonists useful in the present invention include, but are not limited to, AD9677/TAK677 (Dainippon/Takeda), CL-316,243, SB 418790, BRL-37344, L-796568, BMS-196085, BRL-35135A, CGP12177A, BTA-243, Trecadrine, Zeneca D7114, and SR 59119A.

Thyroid hormone 4 agonists useful in the present invention include, but are not limited to, those disclosed in: PCT Application No. WO 02/15845; and Japanese Patent Application No. JP 2000256190.

Specific thyroid hormone β agonists useful in the present invention include, but are not limited to, KB-2611 (KaroBioBMS).

Specific fatty acid synthase (FAS) inhibitors useful in the present invention, include, but are not limited to, Cerulenin and C75.

Lipase inhibitors useful in the present invention include, but are not limited to, those disclosed in: PCT Application No. WO 01/77094. Specific lipase inhibitors useful in the present invention include, but are not limited to, orlistat (Xenical®), Triton WR1339, RHC80267, lipstatin, tetrahydrolipstatin, teasaponin, and diethylumbelliferyl phosphate.

Topiramate (Topimax®), indicated as an anti-convulsant and an anti-convulsant, has been shown to increase weight loss. Metformin (Glucophage®) is indicated for patients with non-insulin dependent diabetes mellitus, particularly those with refractory obesity. Physician's Desk Reference® page 1080-1086, (56^{th} ed. 2002).

Specific bombesin (BRS-3) agonists useful in the present invention, include, but are not limited to, [D-Phe6,beta-Ala 11,Phe 13,Nle 14]Bn(6-14) and [D-Phe6,Phe 13]Bn(6-13)propylamide, and those compounds disclosed in Pept. Sci. 2002 August; 8(8): 461-75).

Zonisamide, a marketed antiepileptic drug with serotonergic and dopaminergic activity in addition to the ability to block sodium and calcium channels, has been shown to result in weight loss in epileptic adults and in obese adults.

The above compounds are only illustrative of the neurokinin-1 antagonists, the anti-obesity agents and the sexual dysfunction therapeutic agents that can be used in the compositions of the present invention. As this listing of compounds is not meant to be comprehensive, the methods of the present invention may employ any neurokinin-1 antagonist, any anti-obesity agent, and any sexual dysfunction therapeutic agent, and are not limited to any particular structural class of compounds.

The present invention also relates to the treatment of obesity, obesity related disorders and sexual dysfunction with a combination of a neurokinin-1 antagonist, and an anti-obesity agent which may be administered separately, therefore the invention also relates to combining separate pharmaceutical compositions or formulations into a kit form. The kit, according to this invention, comprises two separate pharmaceutical compositions: a first unit dosage form comprising a prophylactically or therapeutically effective amount of a neurokinin-1 antagonist, or a pharmaceutically acceptable salt or ester thereof, and a pharmaceutically acceptable carrier or diluent; and a second unit dosage form comprising a prophylactically or therapeutically effective amount of an anti-obesity agent or a sexual dysfunction therapeutic agent, or a pharmaceutically acceptable salt or ester thereof; and a pharmaceutically acceptable carrier or diluent, and a container. The routes of administration, frequency of administration and times of administration of the unit dosage forms within the kit may differ. The kit further comprises a container. Such kits are especially suited for the delivery of solid oral forms such as tablets or capsules, as well as nasal sprays and injectable formulations. Such a kit preferably includes a number of unit dosages. Such kits can include a card having the dosages oriented in the order of their intended use. One example of a kit is a twin pack. Another example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days or time in the treatment schedule in which the dosages can be administered. Throughout the instant application, the following terms have the indicated meanings. The alkyl groups specified above are intended to include those alkyl groups of the designated length in either a straight or branched configuration. Exemplary of such alkyl groups are methyl, ethyl, propyl, isopropyl, butyl, sec-butyl, tertiary butyl, pentyl, isopentyl, hexyl, isohexyl, and the like.

The term “halogen” is intended to include the halogen atoms fluorine, chlorine, bromine and iodine.

The term “C₁₋₄ alkyliminoyl” means C₁₋₃C(═NH)—.

The term “aryl” includes phenyl and naphthyl.

The term “heteroaryl” includes mono- and bicyclic aromatic rings containing from 1 to 4 heteroatoms selected from nitrogen, oxygen and sulfur. “5- or 6-Membered heteroaryl” represents a monocyclic heteroaromatic ring; examples thereof include thiazole, oxazole, thiophene, furan, pyrrole, imidazole, isoxazole, pyrazole, triazole, thiadiazole, tetrazole, oxadiazole, pyridine, pyridazine, pyrimidine, pyrazine, and the like. Bicyclic heteroaromatic rings include, but are not limited to, benzothiadiazole, indole, benzothiophene, benzofuran, benzimidazole, benzisoxazole, benzothiazole, quinoline, benzotriazole, benzoxazole, isoquinoline, purine, furopyridine and thienopyridine.

The term “5- or 6-membered carbocyclyl” is intended to include non-aromatic rings containing only carbon atoms such as cyclopentyl and cyclohexyl.

The term “5 and 6-membered heterocyclyl” is intended to include non-aromatic heterocycles containing one to four heteroatoms selected from nitrogen, oxygen and sulfur. Examples of a 5 or 6-membered heterocyclyl include piperidine, morpholine, thiamorpholine, pyrrolidine, imidazolidine, tetrahydrofuran, piperazine, and the like.

Certain of the above defined terms may occur more than once in the above formula and upon such occurrence each term shall be defined independently of the other; thus for example, NR⁴R⁴ may represent NH₂, NHCH₃, N(CH₃)CH₂CH₃, and the like.

It will be understood that, as used herein, references to MC4R agonists and neurokinin-1 antagonists, including MC4R agonists of Formulas I and II, compound A, neurokinin-1 antagonists, such as Compound B, are meant to also include the pharmaceutically acceptable salts and esters thereof. The pharmaceutically acceptable salts of the composition of the instant invention include the composition wherein one of the individual components of the composition is in the form of a pharmaceutically acceptable salt, or the composition wherein all of the individual components are in the form of pharmaceutically acceptable salts (wherein the salts for each of the components can be the same or different), or a pharmaceutically acceptable salt of the combined components (i.e., a salt of the composition).

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, lithium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, malonic, mucic, nitric, pamoic, pantothenic, phosphoric, propionic, succinic, sulfuric, tartaric, p-toluenesulfonic acid, trifluoroacetic acid, and the like. Particularly preferred are citric, fumaric, hydrobromic, hydrochloric, maleic, phosphoric, sulfuric, and tartaric acids. It will be understood that, as used herein, references to the compounds of Formula I, formula II, compound A, and other melanocortin 4 receptor agonists, are meant to also include the pharmaceutically acceptable salts, such as the hydrochloride salts.

The compounds useful in the methods of the present invention include stereoisomers, such as optical isomers, diastereomers and geometerical isomers, or tautomers depending on the mode of substitution. The compounds may contain one or more chiral centers and occur as racemates, racemic mixtures and as individual diastereomers, enantiomeric mixtures or single enantiomers, keto-enol tautomers, or E and Z olefinic geometric isomers, with all isomeric forms being included in the present invention. The present invention is meant to comprehend all such isomeric forms of the compounds in the compositions of the present invention, and their mixtures. Therefore, where a compound is chiral, the separate enantiomers, substantially free of the other, are included within the scope of the invention; further included are all mixtures of the two enantiomers. Also included within the scope of the invention are polymorphs, hydrates and solvates of the compounds of the instant invention.

The present invention includes within its scope prodrugs of the compounds in the compositions of this invention. In general, such prodrugs will be functional derivatives of the compounds in these compositions which are readily convertible in vivo into the required compound. Thus, in the methods of treatment of the present invention, the term “administering” shall encompass the treatment of obesity, obesity-related disorders, and sexual dysfunction with the compounds specifically disclosed as elements of the composition or with compounds which may not be specifically disclosed, but which convert to the specified compounds in vivo after administration to the patient. Conventional procedures for the selection and preparation of suitable prodrug derivatives are described, for example, in “Design of Prodrugs,” ed. H. Bundgaard, Elsevier, 1985.

The compositions of the present invention are useful for the treatment or prevention of disorders associated with excessive food intake, such as obesity, obesity-related disorders, and sexual dysfunction, including male erectile dysfunction and female sexual dysfunction. The obesity herein may be due to any cause, whether genetic or environmental.

The obesity-related disorders herein are associated with, caused by, or result from obesity. Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovary disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia. Further examples of obesity-related disorders are metabolic syndrome, also known as syndrome X, insulin resistance syndrome, reproductive hormone abnormalities, sexual and reproductive dysfunction, such as impaired fertility, infertility, hypogonadism in males and hirsutism in females, fetal defects associated with maternal obesity, gastrointestinal motility disorders, such as obesity-related gastro-esophageal reflux, respiratory disorders, such as obesity-hypoventilation syndrome (Pickwickian syndrome), breathlessness, cardiovascular disorders, inflammation, such as systemic inflammation of the vasculature, arteriosclerosis, hypercholesterolemia, hyperuricaemia, lower back pain, gallbladder disease, gout, kidney cancer, and increased anesthetic risk. The compositions of the present invention are also useful for reducing the risk of secondary outcomes of obesity, such as reducing the risk of left ventricular hypertrophy. The compositions of the present invention are also useful to treat Alzheimer's disease.

The term “metabolic syndrome”, also known as syndrome X, is defined in the Third Report of the National Cholesterol Education Program Expert Panel on Detection, Evaluation and Treatment of High Blood Cholesterol in Adults (ATP-III). E. S. Ford et al., JAMA, vol. 287 (3), Jan. 16, 2002, pp 356-359. Briefly, a person is defined as having metabolic syndrome if the person has three or more of the following symptoms: abdominal obesity, hypertriglyceridemia, low HDL cholesterol, high blood pressure, and high fasting plasma glucose. The criteria for these are defined in ATP-M.

The term “diabetes,” as used herein, includes both insulin-dependent diabetes mellitus (i.e., IDDM, also known as type I diabetes) and non-insulin-dependent diabetes mellitus (i.e., NIDDM, also known as Type II diabetes). Type I diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese. The compositions of the present invention are useful for treating both Type I and Type II diabetes. The compositions are especially effective for treating Type II diabetes. The compounds or combinations of the present invention are also useful for treating and/or preventing gestational diabetes mellitus.

“Obesity” is a condition in which there is an excess of body fat. The operational definition of obesity is based on the Body Mass Index (BMI), which is calculated as body weight per height in meters squared (kg/m²). “Obesity” refers to a condition whereby an otherwise healthy subject has a Body Mass Index (BMI) greater than or equal to 30 kg/m², or a condition whereby a subject with at least one co-morbidity has a BMI greater than or equal to 27 kg/m². An “obese subject” is an otherwise healthy subject with a Body Mass Index (BMI) greater than or equal to 30 kg/m² or a subject with at least one co-morbidity with a BMI greater than or equal to 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m².

The increased risks associated with obesity occur at a lower Body Mass Index (BMI) in Asians. In Asian countries, including Japan, “obesity” refers to a condition whereby a subject with at least one obesity-induced or obesity-related co-morbidity, that requires weight reduction or that would be improved by weight reduction, has a BMI greater than or equal to 25 kg/m². In Asian countries, including Japan, an “obese subject” refers to a subject with at least one obesity-induced or obesity-related co-morbidity that requires weight reduction or that would be improved by weight reduction, with a BMI greater than or equal to 25 kg/m². In Asia-Pacific, a “subject at risk of obesity” is a subject with a BMI of greater than 23 kg/m² to less than 25 kg/m².

As used herein, the term “obesity” is meant to encompass all of the above definitions of obesity.

Obesity-induced or obesity-related co-morbidities include, but are not limited to, diabetes, non-insulin dependent diabetes mellitus—type II (2), impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, metabolic syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

“Treatment” (of obesity and obesity-related disorders) refers to the administration of the compounds or combinations of the present invention to reduce food intake, to reduce body weight, or to maintain the body weight of an obese subject. One outcome of treatment may be reducing the body weight of an obese subject relative to that subject's body weight immediately before the administration of the compounds or combinations of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. Another outcome of treatment may be to maintain weight loss. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

“Prevention” (of obesity and obesity-related disorders) refers to the administration of the compounds or combinations of the present invention to reduce food intake, to reduce body weight, or to maintain the body weight of a subject at risk of obesity. One outcome of prevention may be reducing the body weight of a subject at risk of obesity relative to that subject's body weight immediately before the administration of the compounds or combinations of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Another outcome of prevention may be to prolong resistance to weight gain. Another outcome of prevention may be to prevent weight regain. Moreover, if treatment is commenced in already obese subjects, such treatment may prevent the occurrence, progression or severity of obesity-related disorders, such as, but not limited to, arteriosclerosis, Type II diabetes, polycystic ovarian disease, cardiovascular diseases, osteoarthritis, dermatological disorders, hypertension, insulin resistance, metabolic syndrome, hypercholesterolemia, hypertriglyceridemia, and cholelithiasis.

Sexual dysfunctions cause marked distress and interpersonal difficulty. The sexual dysfunctions include sexual desire disorders, sexual arousal disorders, orgasmic disorders and sexual pain disorders. None of these disorders is adequately treated with existing therapies.

“Male sexual dysfunction” includes impotence, loss of libido, and erectile dysfunction.

“Erectile dysfunction” is a disorder involving the failure of a male mammal to achieve erection, ejaculation, or both. Symptoms of erectile dysfunction include an inability to achieve or maintain an erection, ejaculatory failure, premature ejaculation, or inability to achieve an orgasm. An increase in erectile dysfunction and sexual dysfunction can have numerous underlying causes, including but not limited to (1) aging, (b) an underlying physical dysfunction, such as trauma, surgery, and peripheral vascular disease, and (3) side-effects resulting from drug treatment, depression, and other CNS disorders.

Treatment of male sexual dysfunction refers to the administration of a compound or combination of the present invention to treat impotence and/or loss of libido, and/or erectile dysfunction in a male mammal in need thereof. One outcome of treatment may be a decrease in impotence. Another outcome of treatment may be an increase in libido. Yet another outcome of treatment may be a decrease in the magnitude or frequency of erectile dysfunction. Treatment of male sexual dysfunction refers to the administration of a compound or combination of the present invention to treat one or more of the symptoms of male sexual dysfunction in a male mammal in need thereof. One outcome of treatment may be increasing the ability to achieve an erection. Another outcome of treatment may be increasing the ability to maintain an erection. Another outcome of treatment may be reducing ejaculatory failure. Another outcome of treatment may be decreasing premature ejaculation. Yet another outcome of treatment may be increasing the ability to achieve an orgasm. Prevention of male sexual dysfunction and male erectile dysfunction refers to the administration of the compounds or combinations of the present invention to prevent the symptoms of sexual dysfunction and erectile dysfunction in a male mammal at risk thereof.

“Female sexual dysfunction” can be seen as resulting from multiple components including dysfunction in desire, sexual arousal, sexual receptivity, and orgasm related to disturbances in the clitoris, vagina, periurethral glans, and other trigger points of sexual function. In particular, anatomic and functional modification of such trigger points may diminish the orgasmic potential in breast cancer and gynecologic cancer patients. Treatment of female sexual dysfunction with an MC-4 receptor agonist can result in improved blood flow, improved lubrication, improved sensation, facilitation of reaching orgasm, reduction in the refractory period between orgasms, and improvements in arousal and desire. In a broader sense, “female sexual dysfunction” also incorporates sexual pain, premature labor, and dysmenorrhea. In one embodiment of the present invention, female sexual dysfunction is selected from the group consisting of: female arousal dysfunction, female orgasmic disorder, hypoactive sexual desire disorder, and sexual pain disorder.

The terms “inhibit emesis” or “minimize emesis” mean to decrease emesis in a subject. One outcome of inhibition may be to stop emesis in a subject in need thereof.

The term “reduce emesis and/or nausea” or “reduction of emesis and/or nausea” means to decrease the amount of emesis and/or nausea experienced by a subject relative to the amount of emesis and/or nausea prior to the start of treatment. In one embodiment the amount of emesis experienced by a subject is decreased by at least 10% relative to the amount of emesis prior to the start of treatment. In another embodiment, the amount of emesis experienced by a subject is decreased by at least 35% relative to the amount of emesis prior to the start of treatment. In another embodiment, the amount of emesis experienced by a subject is decreased by at least 44% relative to the amount of emesis experienced prior to the start of treatment. In yet another embodiment, the amount of emesis experienced by a subject is decreased by at least 100% relative to the amount of emesis experienced prior to the start of treatment.

The term “reduce food intake” or “reduction of food intake” means to decrease the amount of food intake by a subject relative to the amount of food intake prior to the start of treatment. In one embodiment the amount of food intake by a subject is decreased by at least 10% relative to the amount of food intake prior to the start of treatment. In another embodiment, the amount of food intake by a subject is decreased by at least 35% relative to the amount of food intake prior to the start of treatment. In another embodiment, the amount of food intake by a subject is decreased by at least 67% relative to the amount of food intake prior to the start of treatment. In yet another embodiment, the amount of food intake by a subject is decreased by at least 70% relative to the amount of food intake prior to the start of treatment.

The term “reduce bodyweight” or “reduction of bodyweight” means to decrease the bodyweight of a subject relative to the bodyweight prior to the start of treatment. In one embodiment the bodyweight of a subject is decreased by at least 1% relative to the bodyweight prior to the start of treatment. In another embodiment, the bodyweight of a subject is decreased by at least 3% relative to the bodyweight prior to the start of treatment. In another embodiment, the bodyweight gain of a subject is reduced by at least 10% relative to the amount of bodyweight prior to the start of treatment. In yet another embodiment, the bodyweight gain of a subject is reduced by at least 15% relative to the bodyweight prior to the start of treatment. In yet another embodiment, the bodyweight gain of a subject is reduced by at least 10% relative to the amount of bodyweight prior to the start of treatment. In yet another embodiment, the bodyweight gain of a subject is reduced by at least 25% relative to the bodyweight prior to the start of treatment.

The term “selective” means having an activation preference for a specific receptor over other receptors which can be quantified based upon whole cell, tissue, or organism assays which demonstrate receptor activity, such as the cAMP Functional Assay and the Binding Affinity Assay. The compounds of the present invention interact preferentially (i.e. selectively) with the MC4 receptor relative to the other melanocortin receptors. Selectivity for the MC4 receptor is beneficial for compounds administered to humans or mammals, to minimize the number of side effects associated with their administration. MC4 selectivity of a compound over another MC receptor is defined herein as the EC₅₀, or IC₅₀, of the compound at the MC receptor being referenced over the EC₅₀, or IC₅₀, of the compound for the MC4 receptor. As used herein, unless indicated otherwise, use of the term “selective over the other MC receptors” means selective with respect to the other melanocortin receptors, including the MC-1, MC-2, MC-3 and MC-5 receptors. For example, a compound having an EC₅₀ of 8 nM at the MC4 receptor and an EC₅₀ of ≧80 nM at the MC-1, MC-2 MC-3, and MC-5 receptors has a selectivity ratio for the MC4 receptor over the other MC receptors of at least 1:10. Additionally, the term “selective” may also refer to one of the MC-1, MC-2, MC-3 or MC-5 receptors individually. For example, a compound having an EC₅₀ of 8 nM at the MC4 receptor and an EC₅₀ of 80 nM at the MC-1 receptor has a selectivity ratio for the MC4 receptor over the MC-1 receptor of 1:10. Such a compound is selective over the MC-1 receptor regardless of its EC₅₀ value for the MC-2R or MC-5R. For example, the selectivity of a compound for the MC4R over the MC-1R is defined as: MC4R functional selectivity, EC₅₀=[EC₅₀ MC-1R]/[EC₅₀ MC4R], or MC4R binding selectivity, IC₅₀=[IC₅₀ MC-1R]/[IC₅₀ MC4R]. A compound is defined herein as being “selective” for the MC4 receptor when the above mentioned ratio is at least 10, preferably at least 65, and more preferably at least about 100.

The terms “administration of” and or “administering a” composition should be understood to mean providing a composition of the invention to a subject in need of treatment or prevention.

The instant compositions include administration of a single dosage formulation which contains a neurokinin-1 antagonist in combination with an anti-obesity agent or sexual dysfunction therapeutic agent, as well as administration of each of the two active agents (neurokinin-1 antagonist and an anti-obesity agent, such as a melanocortin 4 receptor agonist; or neurokinin-1 antagonist and a sexual dysfunction therapeutic agent, such as a melanocortin 4 agonist) in its own separate dosage formulation. A single dosage formulation will provide convenience for the patient, which is an important consideration especially for patients with diabetes or obese patients who may be in need of multiple medications. However, separate dosage formulations and routes of administration may be required. The present invention also includes administration of two separate dosage formulations at different times, at different dosages and in different frequencies. The separate dosage formulations may be given at different times of the day depending on the duration of action of the individual components. Where separate dosage formulations are used, the individual components of the composition may be administered at essentially the same time, i.e., concurrently, or at separately staggered times, i.e. sequentially prior to or subsequent to the administration of the other component of the composition. Administration in these various ways are suitable for the present compositions as long as the beneficial pharmaceutical effect of the combination of a neurokinin-1 receptor antagonist, and the anti-obesity agent or the sexual dysfunction therapeutic agent, is realized by the patient at substantially the same time. Such beneficial effect is preferably achieved when the target blood level concentrations of each active drug are maintained at substantially the same time. It is preferred that the combination of a neurokinin-1 antagoinst, and the anti-obesity agent or sexual dysfunction therapeutic agent, be co-administered concurrently on a once-a-day dosing schedule; however, varying dosing schedules, such as the neurokinin-1 antagonist once, twice, three times or more per day, and the anti-obesity agent or sexual dysfunction therapeutic agent once, twice, three times or more times per day, are also encompassed herein. An effective amount of the neurokinin-1 antagonist can be administered in a single dose, or in multiple doses, for example two to six doses daily, during a course of treatment. In another embodiment, the neurokinin-1 antagonist is administered whenever the effect (e.g., decreased food intake, weight loss, or reduction in emesis) is desired. In another embodiment, the neurokinin-1 antagonist is administered slightly prior to whenever the effect is desired, such as, but not limited to about 10 minutes, about 15 minutes, about 30 minutes, about 45 minutes, about 60 minutes, about 90 minutes, or about 120 minutes, prior to the time the effect is desired. In another embodiment, a time release formulation is utilized. In another embodiment, a therapeutically effective amount of the neurokinin-1 antagonist is administered as a single pulse dose, as a bolus dose, or as pulse doses administered over time. In another embodiment, the anti-obesity or sexual dysfunction therapeutic agent is administered whenever the effect (e.g., decreased food intake, weight loss, or sexual function) is desired. In another embodiment, the anti-obesity or sexual dysfunction therapeutic agent is administered slightly prior to whenever the effect is desired, such as, but not limited to about 10 minutes, about 15 minutes, about 30 minutes, about 45 minutes, about 60 minutes, about 90 minutes, or about 120 minutes, prior to the time the effect is desired. In another embodiment, a time release formulation is utilized.

The instant pharmaceutical composition is therefore to be understood to include all such regimes of simultaneous or alternating treatment, as well as the use of two dosage formulations that require different routes of administration, and the terms “administration” and “administering” are to be interpreted accordingly.

The term “administration” as used herein refers to modes of parenteral and peripheral routes of administration which include oral, intravenous, intramuscular, intraperitoneal, intrasternal, transdermal, sublingual, buccal, inhaled, subcutaneous and intraarticular injection, infusion and intranasal administration.

The term “subject” as used herein means a mammal. One embodiment of the term “mammal” is a “human,” said human being either male or female. As such, the term “mammal” includes, but is not limited to, companion animals such as cats and dogs, as well as horses.

The term “subject in need thereof” refers to a subject who is in need of treatment or prophylaxis as determined by a researcher, veterinarian, medical doctor or other clinician. In one embodiment, the subject in need of treatmentor prophylaxsis is an obese mammal. In another embodiment, the subject in need of treatmentor prophylaxsis is an obese human with one or more obesity-related co-morbidities. In another embodiment, the subject in need of treatmentor prophylaxsis is an obese human without obesity-related co-morbidities. In another embodiment, the subject in need of treatment or prophylaxsis suffers from sexual dysfunction. In another embodiment, the subject in need of treatment or prophylaxis suffers from male erectile dysfunction. In another embodiment, the subject in need of treatment or prophylaxsis suffers from female sexual dysfunction. In another embodiment, the subject in need of treatment or prophylaxsis suffers from emesis, due to any cause, including treatment with an anti-obesity agent, such as a melanocortin 4 receptor agonist, and treatment with a sexual dysfunction therapeutic, such as MT-II, PT-141, PT-14, apomorphine, and sildenifil.

The administration of the composition of the present invention in order to practice the present methods of therapy is carried out by administering a therapeutically effective amount of the compounds in the composition to a subject in need of such treatment or prophylaxis. The need for a prophylactic administration according to the methods of the present invention is determined via the use of well known risk factors. The effective amount of an individual compound is determined, in the final analysis, by the physician in charge of the case, but depends on factors such as the exact disease to be treated, the severity of the disease and other diseases or conditions from which the patient suffers, the chosen route of administration, other drugs and treatments which the patient may concomitantly require, and other factors in the physician's judgment.

The term “therapeutically effective amount” as used herein means the amount of the active compounds in the composition that will elicit the biological or medical response in a tissue, system, subject, or human that is being sought by the researcher, veterinarian, medical doctor or other clinician, which includes alleviation of the symptoms of the disorder being treated, including obesity, an obesity-related disorder, sexual dysfunction, including male erectile dysfunction, and emesis in subjects at risk for obesity or the obesity-related disorder. The novel methods of treatment of this invention are for disorders known to those skilled in the art.

The term “prophylactically effective amount” as used herein means the amount of the active compounds in the composition that will elicit the biological or medical response in a tissue, system, subject, or human that is being sought by the researcher, veterinarian, medical doctor or other clinician, to prevent the onset of obesity, an obesity-related disorder, sexual dysfunction, including male erectile dysfunction, and emesis in subjects at risk for obesity, an obesity-related disorder or sexual dysfunction.

The magnitude of prophylactic or therapeutic dose of the active ingredients (e.g. neurokinin-1 antagonist, anti-obesity agent or sexual dysfunction therapeutic agent, such as a melanocortin 4 agonist) of the composition will, of course, vary with the nature of the severity of the condition to be treated and with the particular compound in the composition and its route of administration. It will also vary according to the age, weight and response of the individual patient. In general, the daily dose range of each compound in the combination lies within the range of from about 0.0001 mg/kg to about 100 mg/kg, preferably from about 0.001 mg/kg to about 50 mg/kg body weight of a subject in single or divided doses. On the other hand, it may be necessary to use dosages outside these limits in some cases.

For use where a composition for intravenous administration is employed, a suitable dosage range is from about 0.0001 mg/kg to about 50 mg/kg, preferably from 0.001 mg/kg to about 20 mg/kg of each compound in the composition per day.

In the case where an oral composition is employed, a suitable dosage range is, e.g. from about 0.001 mg/kg to about 100 mg/kg of each compound in the composition per day, preferably from about 0.01 mg to about 1000 mg per day. For oral administration, the compositions are preferably provided in the form of tablets containing from 0.01 mg to 1,000 mg, preferably 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 and 1,000 milligrams of each active ingredient for the symptomatic adjustment of the dosage to the subject to be treated. This dosage regimen may be adjusted to provide the optimal therapeutic response.

The compounds of this invention can be administered to humans in the dosage ranges specific for each compound.

In general, for reducing food intake, reducing body weight, maintaining body weight reduction, treating or preventing obesity and/or an obesity related disorder, such as diabetes mellitus and/or metabolic syndrome, and/for treating or preventing sexual dysfunction, the Mc4r agonist of the present invention, including Compound A, is administered at a daily dosage of from about 0.001 milligram to about 100 milligram per kilogram of animal body weight, preferably given in a single dose or in divided doses two to six times a day, or in sustained release form.

In general, for reducing food intake, reducing body weight, maintaining body weight reduction, treating or preventing obesity and an obesity related disorder, such as diabetes mellitus and/or metabolic syndrome, and treating or preventing sexual dysfunction, and emesis caused by the treatment of these disorders, the neurokin-1 antagonist, such as Compound B, is administered at a daily dosage of from about 0.1 mg/kg to about 25 mg/kg of body weight orally. More specifically, when treating or preventing obesity, obesity related disorders, and sexual dysfunction, and emesis caused by the treatment of these disorders, generally satisfactory results may be obtained when a neurokinin-1 antagonist, including Compound B and Compound F, or a pharmaceutically acceptable salt or ester thereof, is administered at a daily oral dosage of from about 0.1 μg/kg to about 25 mg/kg of bodyweight, preferably from about 0.5 μg/kg to about 5 mg/kg of body weight of body weight, and more preferably 1 μg/kg to about 5 mg/kg of body weight, given in a single dose or in divided doses two to six times a day, or in sustained release form.

Leptin may be administered at a daily dosage of from about 0.01 mg/kg to about 20 mg/kg, preferably, from about 0.01 mg/kg to about 0.3 mg/kg, preferably injected in a single dose or in divided doses.

Metformin may be administered at a daily dosage of from about 0.01 mg/kg to about 100 mg/kg, preferably from about 1 mg/kg to about 50 mg/kg in a single dose or in divided doses 2 to 3 times per day, or in sustained release form; more preferably the daily dose is 500 mg, 850 mg, 1000 mg, 1500 mg, 2000 mg or 2550 mg orally given as a single dose or in divided doses 2 to 3 times per day.

Nalmefene may be administered at a daily dosage of from about 0.0001 mg/kg to about 10 mg/kg, preferably from about 0.001 to about 0.05 mg/kg.

Orlistat may be administered at a daily dosage of from about 20 mg to about 1200 mg, preferably from about 120 mg to 400 mg in a single dose or divided doses 2 to 3 times per day or in sustained release form; more preferably a 120 mg single dose 3 times per day, or in sustained release form.

Sibutramine may be administered at a daily dosage of from about 0.01 mg/kg to about 10 mg/kg, preferably from about 0.01 mg/kg to about 1 mg/kg in a single dose or in divided doses 2 to 3 times per day, or in sustained release form; more preferably the single daily dose of sibutramine is 5 mg, 10 mg, 15 mg, 20 mg or 30 mg orally.

Rimonabant may be administered at a daily dosage of from about 0.01 mg/kg to about 8 mg/kg, more preferably from about 0.3 mg/kg to about 3 mg/kg of body weight in a single dose or in divided doses 2 to 3 times per day, or in sustained release form.

Topiramate (Topamax®) may be administered at a daily dosage of from about 10 mg to about 1,600 mg per day, preferably from about 50 mg to about 400 mg per day in a single dose or in divided doses, or in sustained release form.

Zonasamide may be administered at a daily dosage of from about 10 mg to about 1,500 mg per day, preferably from about 100 mg to about 600 mg per day in a single dose or in divided doses, or in sustained release form. More preferably zonasamide may be administered at a daily dosage of from about 100 mg/d orally, with gradual increase to 400 mg/d and further increase to 600 mg/d for patients losing less than 5% of body weight at the end of 12 weeks.

The weight ratio of the neurokinin antagonist to the anti-obesity agent and sexual dysfunction therapeutic agent, including melanocortin 4 receptor agonist of Formula I and II and Compound A, may be varied and will depend upon the effective dose of each ingredient. Generally, an effective dose of each will be used. Thus, for example, when a neurokinin-1 antagonist, such as Compound B, is combined with an anti-obesity agent, such as Compound A, the weight ratio of the neurokinin-1 antagonist to the anti-obesity agent will generally range from about 1000:1 to about 1:1000, preferably about 200:1 to about 1:200. Compositions of the neurokinin antagonist and the anti-obesity agents will generally also be within the aforementioned range, but in each case, an effective dose of each active ingredient should be used.

The effective dosage of each of the active ingredients employed in the composition may vary depending on the particular compound employed, the mode of administration, the condition being treated and the severity of the condition being treated. Thus, the dosage regimen utilizing the compositions of the present invention is selected in accordance with a variety of factors including type, species, age, general health, body weight, diet, sex and medical condition of the subject; the severity of the condition to be treated; the renal and hepatic function of the patient; the drug combination; and the particular compounds employed and their routes of administration. A physician, clinician or veterinarian of ordinary skill can readily determine and prescribe the effective amount of the drug required to prevent, counter or arrest the progress of the condition.

Another aspect of the present invention provides pharmaceutical compositions comprising a pharmaceutical carrier and a therapeutically effective amount of each compound in the composition of the present invention. The term “composition”, as in pharmaceutical composition, is intended to encompass a product comprising the active ingredient(s), and the inert ingredient(s), such as pharmaceutically acceptable excipients, that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a neurokinin-1 antagonist, additional active ingredient(s) such as an anti-obesity agent or a sexual dysfunction therapeutic agent, such as a melanocortin 4 agonist, and pharmaceutically acceptable excipients.

Any suitable route of administration may be employed for providing a subject, especially a human, with an effective dosage of a composition of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a combination of a neurokinin-1 antagonist and an anti-obesity agent or a sexual dysfunction therapeutic agent, such as a melanocortin 4 agonist, as active ingredients or a pharmaceutically acceptable salt or ester thereof, and may also contain a pharmaceutically acceptable carrier and optionally other therapeutic ingredients. By “pharmaceutically acceptable” it is meant the carrier, diluent or excipient must be compatible with the other ingredients of the formulation and not deleterious to the recipient thereof. In particular, the term “pharmaceutically acceptable salts” refers to salts prepared from pharmaceutically acceptable non-toxic bases or acids including inorganic bases or acids and organic bases or acids.

The compositions include compounds suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. These compositions may be conveniently presented in unit dosage form and prepared by any of the methods well-known in the art of pharmacy.

For administration by inhalation, the compositions of the present invention are conveniently delivered in the form of an aerosol spray presentation from pressurized packs or nebulizers. The compositions may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of the instant composition in suitable propellants, such as fluorocarbons or hydrocarbons and dry powder inhalation (DPI) aerosol, which may be formulated as a dry powder of the composition with or without additional excipients.

Suitable topical formulations of the compositions of the present invention include transdermal devices, aerosols, creams, solutions, ointments, gels, lotions, dusting powders, and the like. The topical pharmaceutical compositions containing the compositions of the present invention ordinarily include about 0.005% to 5% by weight of the active compounds in admixture with a pharmaceutically acceptable vehicle. Transdermal skin patches useful for administering the compositions of the present invention include those well known to those of ordinary skill in that art. To be administered in the form of a transdermal delivery system, the dosage administration will, of course be continuous rather than intermittent throughout the dosage regimen.

The compositions of the present invention can also be administered in the form of liposome delivery systems, such as small unilamellar vesicles, large unilamellar vesicles and multilamellar vesicles. Liposomes can be formed from a variety of phospholipids, such as cholesterol, sterylamine or phosphatidylcholines.

Compositions of the present invention may also be delivered by the use of monoclonal antibodies as individual carriers to which the compound molecules are coupled. The compounds in these compositions may also be coupled with soluble polymers as targetable drug carriers. Such polymers can include polyvinylpyrrolidone, pyran copolymer, polyhydroxypropyl-methacrylamide phenol, polyhydroxyethylasparamidepheon, or polyethyleneoxidepolylysine substituted with palmitoyl residues. Furthermore, the compositions of the present invention may be coupled to a class of biodegradable polymers useful in achieving controlled release of a drug, for example, polylactic acid, polyepsilon caprolactone, polyhydroxybutyric acid, polyorthoesters, polyacetals, polydihydropyrans, polycyanoacrylates and cross-linked or amphipathic block copolymers of hydrogels.

Compositions of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

In practical use, each compound in the compositions of the present invention (e.g. neurokinin-1 receptor antagonist or anti-obesity agent, such as melanocortin 4 receptor agonist) can be combined as the active ingredients in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules, pellet, powder and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the composition may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules (including timed release and sustained release formulations), pills, cachets, powders, granules or tablets each containing a predetermined amount of the active ingredients, as a powder or granules or as a solution or a suspension in an aqueous liquid, a non-aqueous liquid, an oil-in-water emulsion or a water-in-oil liquid emulsion, including elixirs, tinctures, solutions, suspensions, syrups and emulsions. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

For example, for oral administration in the form of a tablet, capsule, pellet, or powder, the active ingredient can be combined with an oral, non-toxic, pharmaceutically acceptable inert carrier such as lactose, starch, sucrose, glucose, methyl cellulose, magnesium stearate, mannitol, sorbitol, croscarmellose sodium and the like; for oral administration in liquid form, e.g., elixirs, syrups, slurries, emulsions, suspensions, solutions, and effervescent compositions, the oral drug components can be combined with any oral, non-toxic, pharmaceutically acceptable inert carrier such as ethanol, glycerol, water, oils and the like. Moreover, when desired or necessary, suitable binders, lubricants, disintegrating agents, buffers, coatings, and coloring agents can also be incorporated. Suitable binders can include starch, gelatin, natural sugars such a glucose, anhydrous lactose, free-flow lactose, beta-lactose, and corn sweeteners, natural and synthetic gums, such as acacia, guar, tragacanth or sodium alginate, carboxymethyl cellulose, polyethylene glycol, waxes, and the like. Lubricants used in these dosage forms include sodium oleate, sodium stearate, magnesium stearate, sodium benzoate, sodium acetate, sodium chloride and the like. Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor. When a dosage unit form is a capsule, it may contain, in addition to materials of the above type, a liquid carrier such as a fatty oil.

Desirably, each tablet contains from 0.01 to 1,000 mg, particularly 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 and 1,000 milligrams of each active ingredient in the composition of the present invention (e.g., neurokinin-1 receptor antagonist, anti-obesity agent, such as melanocortin 4 receptor agonist) for the symptomatic adjustment of the dosage to the subject to be treated; and each cachet or capsule contains from about 0.01 to 1,000 mg, particularly 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 and 1,000 milligrams of each active in the composition of the present invention (e.g., neurokinin-1 receptor antagonist, anti-obesity agent, such as melanocortin 4 receptor agonist) for the symptomatic adjustment of the dosage to the subject to be treated.

Exemplifying the invention is a pharmaceutical composition comprising a neurokinin-1 receptor antagonist, and anti-obesity agent, such as a melanocortin 4 receptor agonist, described above, and a pharmaceutically acceptable carrier. Also exemplifying the invention is a pharmaceutical composition made by combining any of the neurokinin-1 antagonists and anti-obesity agents, such as melanocortin 4 agonists, described above and a pharmaceutically acceptable carrier. An illustration of the invention is a process for making a pharmaceutical composition comprising combining any of the neurokinin-1 antagonists and anti-obesity agents, such as melanocortin 4 agonists, described above and a pharmaceutically acceptable carrier.

The dose may be administered in a single daily dose or the total daily dosage may be administered in divided doses of two to six times daily. Furthermore, based on the properties of the individual compound selected for administration, the dose may be administered less frequently, e.g., weekly, twice weekly, monthly, etc. The unit dosage will, of course, be correspondingly larger for the less frequent administration.

When administered via intranasal routes, transdermal routes, by rectal or vaginal suppositories, or through a continual intravenous solution, the dosage administration will, of course, be continuous rather than intermittent throughout the dosage regimen.

The following are examples of representative pharmaceutical dosage forms for the compositions of the present invention:

| TABLET | mg/tablet |
|---|---|
| NK-1 antagonist Compound B | 25 |
| MC4R agonist Compound A | 10 |
| Microcrystalline Cellulose | 40.5 |
| Lactose | 111.5 |
| Croscarmellose Sodium | 5.0 |
| Hydroxypropylcellulose | 6.0 |
| Sodium Dodecyl Sulfate | 1.0 |
| Magnesium Stearate | 1.0 |
| | 200 |

| Capsule | mg/capsule |
|---|---|
| NK-1 antagonist Compound B | 100 |
| MC4R agonist Compound A | 10 |
| Lactose | 70 |
| Sodium Dodecyl Sulfate | 20 |
| | 200 |

| Injectable Suspension (I.M.) | mg/mL |
|---|---|
| NK-1 antagonist Compound B | 0.70 |
| MC4R agonist Compound A | 1.0 |
| cyclodextrin | Q.S. ed to |
| (35% weight/volume) | 1 ml volume |
| glycerol | 63.05 |
| Aerosol | Per canister |
| NK-1 antagonist Compound B | 4 mg |
| MC4R agonist Compound A | 9 mg |
| Lecithin, NF Liq. Conc. | 1.2 mg |
| Trichlorofluoromethane, NF | 4.025 g |
| Dichlorodifluoromethane, NF | 12.15 g |

Water for injection to a total volume of 1 mL

It will be understood that the scope of compositions of the compounds of this invention with other agents useful for treating or preventing obesity, obesity-related disorders, and sexual dysfunction includes in principle any combination with any pharmaceutical composition useful for treating obesity and obesity-related disorders.

In order to illustrate the invention, the following examples are included. These examples do not limit the invention. They are only meant to suggest a method of reducing the invention to practice. Those skilled in the art may find other methods of practicing the invention which are readily apparent to them. However, those methods are also deemed to be within the scope of this invention.

### EXAMPLE 1

### NK-1 Receptor Binding Assay

NK-1 receptor binding assays are performed in intact Chinese hamster ovary (CHO) cells expressing the human NK-1 receptor using a modification of the assay conditions described by Cascieri et al, J. Pharmacol. Exp. Ther., 1992, 42, 458. Typically, the receptor is expressed at a level of 3×10⁵ receptors per cell. Cells are grown in monolayer culture, detached from the plate with enzyme-free dissociation solution (Speciality Media Inc.), and washed prior to use in the assay. ¹²⁵I-Tyr⁸-substance P (0.1 nM, 2000 Ci/mmol; New England Nuclear) is incubated in the presence or absence of test compounds (dissolved in 51 μl dimethylsulphoxide, DMSO) with 5×10⁴ CHO cells. Ligand binding is performed in 0.25 ml of 50 mM Tris-HCl, pH7.5, containing 5 mM MnCl₂, 150 mM NaCl, 0.02% bovine serum albumin (Sigma), 50 μg/ml chymostatin (Peninsula), 0.1 nM phenylmethylsulphonyl fluoride, 2.8 μg/ml pepstatin, 2 μg/ml leupeptin and 2.8 μg/ml furoyl saccharine. The incubation proceeds at room temperature until equilibrium is achieved (>40 minutes) and the receptor-ligand complex is harvested by filtration over GF/C filters pre-soaked in 0.1% polyethylenimine using a Tomtek 96-well harvester. Non-specific binding is determined using excess substance P (1 μM) and represents <10% of total binding.

### EXAMPLE 2

### Gerbil Foot-Tapping Assay

CNS-penetrant NK-1 receptor antagonists for use in the present invention can be identified by their ability to inhibit foot tapping in gerbils induced by anxiogenic agents (such as pentagastrin) or central infusion of NK-1 receptor agonists such as GR73632, or caused by aversive stimulation such as foot shock or single housing, based on the method of Rupniak & Williams, Eur. J. Pharmacol., 1994, 265, 179.

Male or female Mongolian gerbils (35-90 g) are anaesthetised by inhalation of an isoflurane/oxygen mixture to permit exposure of the jugular vein in order to permit administration of test compounds or vehicle in an injection volume of 5 ml/kg i.v. Alternatively, test compounds may be administered orally or by subcutaneous or intraperitoneal routes. A skin incision is then made in the midline of the scalp to expose the skull. An anxiogenic agent (e.g. pentagastrin) or a selective NK-1 receptor agonist (e.g. GR73632 (d Ala[L-Pro⁹, Me-Leu⁸]-substance P-(7-11)) is Leu¹⁰]-substance P-(7-11)) is infused directly into the cerebral ventricles (e.g. 3 μmol in 5 μl i.c.v., depending on test substance) by vertical insertion of a cuffed 27 gauge needle to a depth of 4.5 mm below bregma. The scalp incision is closed and the animal allowed to recover from anaesthesia in a clear perspex observation box (25 cm×20 cm×20 cm). The duration and/or intensity of hind foot tapping is then recorded continuously for approximately 5 minutes. Alternatively, the ability of test compounds to inhibit foot tapping evoked by aversive stimulation, such as foot shock or single housing, may be studied using a similar method of quantification.

### EXAMPLE 3

### Ferret Emesis Assay

Individually housed male ferrets (1.0-2.5 kg) are dosed orally by gavage with test compound. Ten minutes later they are fed with approximately 100 g of tinned cat food. At 60 minutes following oral dosing, cisplatin (10 mg/kg) is given i.v. via a jugular vein catheter inserted under a brief period of halothane anaesthesia. The catheter is then removed, the jugular vein ligated and the skin incision closed. The ferrets recover rapidly from the anaesthetic and are mobile within 10-20 minutes. The animals are observed continuously during recovery from the anaesthetic and for 4 hours following the cisplatin injection, after which time the animals are killed humanely. The numbers of retches and vomits occurring during the 4 hours after cisplatin administration are recorded by trained observers.

### EXAMPLE 4

### Separation-Induced Vocalisation Assay

Male and female guinea-pigs pups are housed in family groups with their mothers and littermates throughout the study. Experiments are commenced after weaning when the pups are 2 weeks old. Before entering an experiment, the pups are screened to ensure that a vigorous vocalisation response is reproducibly elicited following maternal separation. The pups are placed individually in an observation cage (55 cm×39 cm×19 cm) in a room physically isolated from the home cage for 15 minutes and the duration of vocalisation during this baseline period is recorded. Only animals which vocalise for longer than 5 minutes are employed for drug challenge studies (approximately 50% of available pups may fail to reach this criterion). On test days each pup receives an oral dose or an s.c. or i.p. injection of test compound or vehicle and is then immediately returned to the home cage with its mother and siblings for 30 to 60 minutes (or for up to 4 hours following an oral dose, dependent upon the oral pharmacokinetics of the test compound) before social isolation for 15 minutes as described above. The duration of vocalisation on drug treatment days is expressed as a percentage of the pre-treatment baseline value for each animal. The same subjects are retested once weekly for up to 6 weeks. Between 6 and 8 animals receive each test compound at each dose tested.

As used herein, the term “CNS-penetrant” refers to NK-1 receptor antagonists which are able to inhibit NK-1 receptor antagonist-induced foot-tapping in the gerbil as hereinafter defined.

Essentially, hind foot-tapping in the gerbil induced by infusion of the NK-1 receptor agonist, GR73632 (d Ala[L-Pro⁹, Me-Leu⁸]-substance P-(7-11)), under anaesthesia, directly into the central ventricles is inhibited when a CNS-penetrant NK-1 receptor antagonist is administered intravenously immediately prior to GR73632 challenge, wherein hind foot-tapping over a period of five minutes following recovery from the anaesthesia is inhibited with an ID₅₀<3 mg/kg, and preferably with an ID₅₀<1 mg/kg.

In an alternative method, the NK-1 receptor antagonist is administered orally, 1 hour prior to GR73632 challenge, wherein the foot-tapping over a period of five minutes following recovery from anaesthesia is inhibited with an ID₅₀<30 mg/kg, and preferably with an ID₅₀<10 mg/kg.

CNS-penetrant NK-1 receptor antagonists of use in the present invention are also effective in the attenuation of separation-induced vocalisations by guinea-pig pups as hereinafter defined. Essentially, a vocalisation response in guinea-pig pups is induced by isolation from their mothers and littermates, which response is attenuated when a CNS-penetrant NK-1 receptor antagonist is administered subcutaneously 30 minutes prior to isolation, wherein vocalisations during the first 15 minutes of isolation are attenuated with an ID₅₀<20 mg/kg, preferably with an ID₅₀<10 mg/kg, and especially with an ID₅₀<5 mg/kg.

In an alternative method, the NK-1 receptor antagonist is administered orally, 4 hours prior to isolation, wherein vocalisations during the first 15 minutes of isolation are attenuated with an ID₅₀<20 mg/kg, preferably with an ID₅₀<10 mg/kg, and especially with an ID₅₀<5 mg/kg.

### EXAMPLE 5

### Melanocortin 4 Receptor Binding Assay

The membrane binding assay was used to identify competitive inhibitors of ¹²⁵I-NDP-alpha-MSH binding to cloned human MCRs expressed in mouse L- or Chinese hamster ovary (CHO)-cells.

Cell lines expressing melanocortin receptors were grown in T-180 flasks containing selective medium of the composition: 1 L Dulbecco's modified Eagles Medium (DMEM) with 4.5 g L-glucose, 25 mM Hepes, without sodium pyruvate, (Gibco/BR1); 100 ml 10% heat-inactivated fetal bovine serum (Sigma); 10 mL 10,000 unit/mL penicillin & 10,000 μg/mL streptomycin (Gibco/BR1); 10 ml 200 mM L-glutamine (Gibco/BR1); 1 mg/mL geneticin (G418) (Gibco/BR1). The cells were grown at 37° C. with CO₂ and humidity control until the desired cell density and cell number was obtained.

The medium was poured off and 10 mls/T-180 flask of enzyme-free dissociation media (Specialty Media Inc.) was added. The cells were incubated at 37° C. for 10 min or until cells sloughed off when flask was banged against hand.

The cells were harvested into 200 mL centrifuge tubes and spun at 1000 rpm, 4° C., for 10 min. The supernatant was discarded and the cells were resuspended in 5 mls/monolayer membrane preparation buffer having the composition: 10 mM Tris pH 7.2-7.4; 4 μg/mL Leupeptin (Sigma); 10 μM Phosphoramidon (Boehringer Mannheim); 40 μg/mL Bacitracin (Sigma); 5 μg/mL Aprotinin (Sigma); 10 mM Pefabloc (Boehringer Mannheim). The cells were homogenized with motor-driven dounce (Talboy setting 40), using 10 strokes and the homogenate centrifuged at 6,000 rpm, 4° C., for 15 min.

The pellets were resuspended in 0.2 mls/monolayer membrane prep buffer and aliquots were placed in tubes (500-1000 μL/tube) and quick frozen in liquid nitrogen and then stored at −80° C.

Test compounds or unlabelled NDP-α-MSH was added to 100 μL of membrane binding buffer to a final concentration of 1 μM. The membrane binding buffer had the composition: 50 mM Tris pH 7.2; 2 mM CaCl₂; 1 mM MgCl₂; 5 mM KCl; 0.2% BSA; 4 μg/mL Leupeptin (SIGMA); 10 μM Phosphoramidon (Boehringer Mannheim); 40 μg/mL Bacitracin (SIGMA); 5 μg/mL Aprotinin (SIGMA); and 10 mM Pefabloc (Boehringer Mannheim). One hundred μL of membrane binding buffer containing 10-40 μg membrane protein was added, followed by 100 μM 125I-NDP-α-MSH to final concentration of 100 μM. The resulting mixture was vortexed briefly and incubated for 90-120 min at room temp while shaking.

The mixture was filtered with Packard Microplate 196 filter apparatus using Packard Unifilter 96-well GF/C filter with 0.1% polyethyleneimine (Sigma). The filter was washed (5 times with a total of 10 mL per well) with room temperature of filter wash having the composition: 50 mM Tris-HCl pH 7.2 and 20 mM NaCl. The filter was dried, and the bottom sealed and 50 μL of Packard Microscint-20 was added to each well. The top was sealed and the radioactivity quantitated in a Packard Topcount Microplate Scintillation counter.

Melanocortin-4 receptor agonists of use in the present invention are compounds which are potent melanocortin-4 receptor agonists, i.e. compounds with an MC4R affinity (IC₅₀) of less than 300 nM, preferably less than 100 nM, and more preferably less than 45 nM.

Results of binding assay (Example 1) and selectivity for representative compounds of the present invention are provided below:

| | IC₅₀ (nM) | Binding Assay IC₅₀ (nM) | | | Selectivity Binding IC₅₀ | | |
|---|---|---|---|---|---|---|---|
| Compound | hMC4R | hMC1bR | hMC3R | hMC5R | 1R/4R | 3R/4R | 5R/4R |
| A | 44 | 5600 | 7200 | 1600 | 127.27 | 163.6 | 36.36 |
| B | 347 | 7600 | >20000 | 5250 | 21.90 | >57.64 | >15.13 |
| C | 420 | | >20000 | 3900 | | >47.62 | >9.29 |
| D | 182 | | >20000 | 4600 | | >109.89 | >25.27 |
| E | 520 | | >20000 | 6300 | | >38.46 | >12.12 |

### EXAMPLE 6

### cAMP Functional Assay

### To Discriminate Melanocortin Receptor Agonists from Antagonists

Cells (for example, CHO— or L-cells or other eukaryotic cells) expressing a human melanocortin receptor (see e.g. Yang-YK; Ollmann-MM; Wilson-BD; Dickinson-C; Yamada-T; Barsh-GS; Gantz-I; Mol-Endocrinol. 1997 March; 11(3): 274-80) were dissociated from tissue culture flasks by rinsing with Ca and Mg free phosphate buffered saline (14190-136, Life Technologies, Gaithersburg, Md.) and detached following 5 min incubation at 37° C. with enzyme free dissociation buffer (S-014-B, Specialty Media, Lavellette, N.J.). Cells were collected by centrifugation and resuspended in Earle's Balanced Salt Solution (14015-069, Life Technologies, Gaithersburg, Md.) with additions of 10 mM HEPES pH 7.5, 5 mM MgCl₂, 1 mM glutamine and 1 mg/ml bovine serum albumin. Cells were counted and diluted to 1 to 5×10⁶/mL. The phosphodiesterase inhibitor 3-isobutyl-1-methylxanthine was added to cells to 0.6 mM.

Agonist Assay: Test compounds were diluted in dimethylsulfoxide (DMSO) (10⁻⁵ to 10⁻¹⁰ M) and 0.1 volume of compound solution was added to 0.9 volumes of cell suspension; the final DMSO concentration was 1%. After room temperature incubation for 45 min, cells were lysed by incubation at 100° C. for 5 min to release accumulated cAMP.

cAMP was measured in an aliquot of the cell lysate with the Amersham (Arlington Heights, Ill.)

cAMP detection assay (RPA556). The amount of cAMP production which resulted from an unknown compound was compared to that amount of cAMP produced in response to alpha-MSH which was defined as a 100% agonist. The EC₅₀ is defined as the compound concentration which results in half maximal stimulation, when compared to its own maximal level of stimulation.

Antagonist assay: Antagonist activity was defined as the ability of a compound to block cAMP production in response to alpha-MSH or other agonists. Solution of test compounds and suspension of receptor containing cells were prepared and mixed as described above; the mixture was incubated for 15 min, and an EC₅₀ dose (approximately 10 nM alpha-MSH) was added to the cells. The assay was terminated at 45 min and cAMP quantitated as above. Percent inhibition was determined by comparing the amount of cAMP produced in the presence to that produced in the absence of test compound.

Melanocortin-4 receptor agonists of use in the present invention are compounds which are potent melanocortin-4 receptor agonists, i.e. compounds with an MC4R functional activity (EC₅₀) less than 90 nM, preferably less than 40 nM, and more preferably less than 15 nM.

Results of cAMP assay (Example 2) and selectivity for representative compounds of the present invention are provided below:

| | EC₅₀ | | | | | | | | |
|---|---|---|---|---|---|---|---|---|---|
| | (nM) | cAMP Assay EC₅₀ (nM) | | | | Selectivity cAMP EC₅₀ | | | |
| Compound | hMC4R | hMC1bR | hMC2R | hMC3R | hMC5R | 1R/4R | 2R/4R | 3R/4R | 5R/4R |
| A | 14 | 1700 | >10000 | 920 | 1400 | 121.43 | >714.2 | 65.71 | 100 |
| B | 88 | 1804 | | 2967 | >5000 | 20.50 | | 33.72 | >56.8 |
| C | 76 | 1100 | | 2625 | >5000 | 14.47 | | 34.54 | >65.7 |
| D | 27 | 580 | | 1500 | 3600 | 21.48 | | 55.56 | 133.3 |
| E | 120 | 1800 | | >5000 | >5000 | 15.00 | | 41.67 | 41.67 |

### EXAMPLE 7

### In Vivo Study of the Effect of Melanocortin 4 Agonist, Compound A, and Neurokinin-1 Antagonist, Compound B, on the Reduction of Emesis and the Reduction in Food Intake and Body Weight in Ferrets

### Methods

Ad lib fed ferrets were dosed with vehicle (0.5% MC, 0.5% Tween80, 1 ml/kg po), NK1 antagonist, Compound B (10 mg/kg), MC4R agonist, Compound A (60 mg/kg), or the combination of these two compounds orally at approximately 9:30 AM. Food intake (FI) was monitored at 1, 2, 4, and 24, hours after dosing. Body weight (BWT) was measured at the time of dosing and at 24 hours. Any incidence of emesis or abnormal behavior by subjects was recorded. Two studies were combined to generate the data.

### Results

Emesis, behavior, food intake, and body weight data are shown below. Emesis was observed in the MC4R agonist group (1, 2, and 4 hours post dose) and in the NK1antagonist/MC4R agonist combination group (1 and 4 hours post dose). The extent of emesis seen with melanocortin 4 agonist Compound A (77% of ferrets) was decreased when co-administered with neurokinin-1 antagonist, Compound B (33%) (See Table 1). A bodyweight (BWT) decrease was observed for the melanocortin 4 agonist, Compound A, and with the NK1 antagonist/MC4R agonist combination group at 24 hours post dose. There was a significant decrease in food intake (FI) at 1, 2, 4, and 24 hours post dose for the NK1 antagonist/MC4R agonist combination group as well as the MC4R agonist dose group. Food intake was significantly reduced in the NK1 antagonist dose group at 24 hours only. For both food intake and body weight, treatment with the combination of NK1 antagonist and MC4R agonist resulted in a significantly greater decrease than with MC4R agonist treatment alone and NK-1 antagonist alone (See FIGS. 1 and 2). All FI and BWT data were analyzed by two-tailed T-TEST.

TABLE 1
Reduction in MC4R associated Emesis

| | Dose | | | | | |
|---|---|---|---|---|---|---|
| | NK1 antagonist, | | | | Melanocortin 4 receptor | |
| | Compound B | | Compound A + | | agonist, Compound A, | |
| | 10 mg/kg PO | | Compound B | | 60 mg/kg | |
| Time | % Emesis | # of Ferrets | % Emesis | # of Ferrets | % Emesis | # of Ferrets |
| 1 hour | 0% | 0/9 | 17% | 2/12 | 31% | 4/13 |
| 2 hours | 0% | 0/9 | 0% | 0/12 | 8% | 1/13 |
| 4 hours | 0% | 0/9 | 17% | 2/12 | 69% | 9/13****** |
| Total | 0% | 0/9 | 33% | 4/12 | 77% | 10/13**** |

*represents # of animals with more than one incidence of emesis

These results show that the combination of a neurokinin-1 antagonist and an anti-obesity agent, such as a melanocortin 4 agonist, is useful for treating obesity and in reducing the emesis side effect caused by treatment with the anti-obesity agent.

### EXAMPLE 8

### In Vivo Study of the Effect of Melanocortin 4 Agonist, Compound A, and Neurokinin-1 Antagonist Compound B, on the Reduction of Emesis and the Reduction in Food Intake and Body Weight in Ferrets

### Methods

Ad lib fed ferrets were dosed with vehicle (0.5% MC, 0.5% Tween80, 1 ml/kg po), NK1 antagonist, Compound B (10 mg/kg), MC4R agonist, Compound A (60 mg/kg), or the combination of these two compounds orally at approximately 9:30 AM. Food intake (FI) was monitored at 1, 2, 4, and 24, hours after dosing. Body weight (BWT) was measured at the time of dosing and at 24 hours. Any incidence of emesis or abnormal behavior by subjects was recorded. Two studies were combined to generate the data.

### Results

Emesis behavior, food intake, and body weight data are shown below. Emesis was observed in the MC4R angonist group (1, 2, and 4 hours post dose) and in the NK1antagonist/MC4R agonist combination group (1 and 4 hours post dose). The extent of emesis seen with melanocortin 4 agonist Compound A (77% of ferrets) was decreased when co-administered with neurokinin-1 antagonist, Compound B (33%) (See Table 1). A bodyweight (BWT) decrease was observed for the melanocortin 4 agonist, Compound A, and with the NK1 antagonist/MC4R agonist combination group at 24 hours post dose. There was a significant decrease in food intake (FI) at 1, 2, 4, and 24 hours post dose for the NK1 antagonist/MC4R agonist combination group as well as the MC4R agonist dose group. Food intake was significantly reduced in the NK1 antagonist dose group at 24 hours only. For both food intake and body weight, treatment with the combination of NK1 antagonist and MC4R agonist resulted in a significantly greater decrease than with MC4R agonist treatment alone and NK-1 antagonist alone (See FIGS. 1 and 2). All FI and BWT data were analyzed by two-tailed T-TEST.

TABLE 2
Reduction in MC4R associated Emesis

| | Dose | | | | | | | |
|---|---|---|---|---|---|---|---|---|
| | | | Compound F | | Compound F | | | |
| | Compound F | | (0.3 mg/kg) + | | (1 mg/kg) + | | Compound A | |
| | (1 mg/kg, PO) | | Compound A | | Compound A | | (10 mg/kg) | |
| | % | # of | % | # of | % | # of | % | # of |
| time | Emesis | ferrets | Emesis | ferrets | Emesis | ferrets | Emesis | ferrets |
| 1 hour | 0% | 0/5 | 60% | 3/5** | 40% | 2/5* | 40% | 2/5** |
| 2 hours | 0% | 0/5 | 60% | 3/5 | 40% | 2/5 | 40% | 2/5 |
| 4 hours | 0% | 0/5 | 60% | 3/5 | 40% | 2/5 | 60% | 5/5*** |
| total | 0% | 0/5 | 60% | 3/5** | 40% | 2/5** | 100% | 5/5***** |

*represents # of animals with more than one incidence of emesis

These results show that the combination of a neurokinin-1 antagonist and an anti-obesity agent, such as a melanocortin 4 agonist, is useful for treating obesity and in reducing the emesis side effect caused by treatment with the anti-obesity agent.

### EXAMPLE 9

### Male Sexual Dysfunction

### Mouse Electrically Stimulated Cavernosal Nerve (ESCN) Assay

Male C57BL6 mice are anesthetized, the carotid artery is exposed and cannulated for measurement of arterial pressure (MAP). A 30 G needle attached to PE10 tubing, filled with heparinized saline, was inserted into the artery and glued in place. This tubing was connected to a pressure transducer and amplifier to measure direct MAP on a Gould 8 channel oscilloscope connected to a computer using the Po-ne-mah software to collect the data at one minute intervals. Another PE10 line attached to a 30 G needle was inserted into the jugular vein for compound or vehicle administration. The cavernous nerve and penile body were exposed through a midline incision. Surrounding muscles were cauterized and removed for visualization of the cavernous nerve, which arises from the ipsilateral pelvic ganglion and is situated dorsal to the prostate. Another 30 G needle attached to PE10 tubing, filled with heparinized saline, was inserted into the base of the corpus cavernosum near the crura and connected to the Gould system. A slight increase in intercavernous pressure (ICP) of approximately 5 to 10 mmHg is observed once this cannula is inserted into the corpus cavernosum. Heparinized saline (200 units/mL) was flushed through the cannula to assure proper placement of the cannula, inducing tumescence. The cavernous nerve was then isolated using curved #5 Dumont forceps and placed on a modified fixed position bipolar silver electrode (Harvard Apparatus). The electrodes are encased in plastic to allow stimulation of the nerve without additional stimulation of surrounding tissues. The electrode was advanced and held by a micromanipulator and was attached to a square wave stimulator to deliver electrical impulses at stimulation parameters ranging between 0.5 to 6.0 v, 2 to 16 Hz, 1 ms, for 30 seconds. Electrical stimulations were administered to individual animals with 5 minute intervals between stimulations. Responses reported at each time point represent the mean of the two stimulations. ICP, MAP and ICP/MAP responses were continuously recorded at one second intervals for the duration of the experiment.

Measurements of ICP, MAP and ICP/MAP ratio are analyzed and responses compared to nerve stimulation in the presence and absence of compound or vehicle. For each parameter monitored, responses evoked by duplicate electrical stimulations were averaged, and the mean values were used for comparison. Response segments of 10 s of baseline+30 s stimulation+150 s post-stimulation were used to evaluate changes in ICP in response to electrical stimulation of the cavernous nerve. To assess direct effects of compound administration on ICP, a 300 s pre-compound response segment was compared to a comparable segment immediately after compound administration.

Compounds useful in the present invention increase intracavernous pressure by at least 25% for a time period of at least 15 minutes relative to placebo.

### EXAMPLE 10

### Models of Female Sexual Dysfunction

Rodent assays relevant to female sexual receptivity include the behavioral model of lordosis and direct observations of copulatory activity. There is also a urethrogenital reflex model in anesthetized spinally transected rats for measuring orgasm in both male and female rats. These and other established animal models of female sexual dysfunction are described in McKenna K E et al, A Model For The Study of Sexual Function In Anesthetized Male And Female Rats, Am. J. Physiol. (Regulatory Integrative Comp. Physiol 30): R1276-R1285, 1991; McKenna K E et al, Modulation By Peripheral Serotonin of The Threshold For Sexual Reflexes In Female Rats, Pharm. Bioch. Behav., 40:151-156, 1991; and Takahashi L K et al, Dual Estradiol Action In The Diencephalon And The Regulation Of Sociosexual Behavior In Female Golden Hamsters, Brain Res., 359:194-207, 1985.

### EXAMPLE 11

### Human Study for Combination Therapy with a Neurokinin-1 Antagonist Such as Compound B, and an Anti-Obesity Agent, Such as Melanocortin 4 Agonist, Compound A

### Materials and Methods

800 people with a BMI>30 are advised to diet and increase their physical activity. After a two-week placebo run-in period, which includes a standardized program of diet, physical activity, and lifestyle changes, the patients are randomized into 4 treatment groups: 1) placebo; 1) an effective dose of neurokinin-1 antagonist, Compound B, such as 10 mg given once a day; 3) an effective dose of a second anti-obesity agent, such as 10 mg of melanocortin 4 agonist, Compound A, given once a day; and 4) an effective dose of Compound A plus an effective dose of Compound B. The neurokinin-1 antagonist and melanocortin 4 agonist are given in tablet form at once per day, as previously determined to be effective. Patients are treated for 6 months, body weights are measured weekly, and appetite, hunger, satiety are measured weekly using standard questionnaires.

Effective anti-obesity combinations result in a greater body weight change when the neurokinin-1 antagonist and the anti-obesity agent, such as the melanocortin 4 agonist, are given together, than the body weight change seen with either compound is administered alone.

### EXAMPLES OF PHARMACEUTICAL COMPOSITIONS

As a specific embodiment of an oral composition of a composition of the present invention, 5 mg of Example 3 is formulated with sufficient finely divided lactose to provide a total amount of 580 to 1000 mg to fill a size O hard gelatin capsule.

As another specific embodiment of an oral composition of a compound of the present invention, 2.5 mg of Example 4 is formulated with sufficient finely divided lactose to provide a total amount of 580 to 1000 mg to fill a size O hard gelatin capsule.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the subject being treated for any of the indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1-24. (canceled)

25. A composition comprising
(a) a neurokinin-1 antagonist, or a pharmaceutically acceptable salt or ester thereof; and
(b) an anti-obesity agent, or a pharmaceutically acceptable salt or ester thereof,
provided that the anti-obesity agent is not selected from the group consisting of: serotonin agonist, selective serotonin reuptake inhibitor, fluvoxamine, paroxetine, sertraline, a minorex, amphechloral, amphetamine, benzphetamine, p-chloroamphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, fluoxetine, furfurylmethyl-amphetamine, levamfetamine, levophacetoperane, mazindol, mefenorex, metamfepramone, methamphetamine, norpseudoephedrine, pentorex, phendimetrazine, phenmetrazine, phentermine, phenylpropanolamine, picilorex and sibutramine; and pharmaceutically acceptable salts thereof.

26. The composition of claim 25 wherein the anti-obesity agent is selected from the group consisting of:
(1) CB-1 antagonist/inverse agonist;
(2) ghrelin antagonist;
(3) H3 antagonist/inverse agonist;
(4) MCH1R antagonist;
(5) MCH2R agonist/antagonist;
(6) MC3R agonist;
(7) MC4R agonist;
(8) Neuromedin U 1 receptor agonist;
(9) Neuromedin U 2 receptor agonist;
(10) NPY1 antagonist;
(11) NPY2 agonist;
(12) NPY4 agonist;
(13) NPY5 antagonist;
(14) leptin;
(15) leptin agonist/modulator;
(16) leptin derivatives;
(17) opioid antagonist;
(18) orexin antagonist;
(19) BRS3 agonist;
(20) 11β HSD-1 inhibitor;
(21) CCK-A agonist;
(22) CNTF;
(23) CNTF agonist/modulator;
(24) CNTF derivative;
(25) DP-IV inhibitor;
(26) GHS agonist;
(27) UCP-1, 2, and 3 activator;
(28) β3 agonist;
(29) thyroid hormone β agonist;
(30) FAS inhibitor;
(31) DGAT1 inhibitor;
(32) DGAT2 inhibitor;
(33) ACC2 inhibitor;
(34) glucocorticoid antagonist;
(35) acyl-estrogens;
(36) lipase inhibitor;
(37) fatty acid transporter inhibitor;
(38) dicarboxylate transporter inhibitor;
(39) glucose transporter inhibitor;
(40) GLP-1 agonist;
(41) axokine;
(42) metformin;
(43) nalmefene;
(44) phytopharm compound 57;
(45) topiramate; and
(46) zonisamide;
or a pharmaceutically acceptable salt or ester thereof.

27. The composition of claim 25 wherein the anti-obesity agent is a melanocortin 4 receptor agonist, or a pharmaceutically acceptable salt or ester thereof.

28. A composition comprising
(a) a neurokinin-1 antagonist, or a pharmaceutically acceptable salt or ester thereof; and
(b) a sexual dysfunction therapeutic agent, or a pharmaceutically acceptable salt or ester thereof.

29. The composition of claim 28 wherein the sexual dysfunction therapeutic agent is selected from the group consisting of:
(1) a type V cyclic-GMP-selective phosphodiesterase inhibitor;
(2) an α₁-adrenergic receptor antagonist;
(3) an α₂-adrenergic receptor antagonist;
(4) a dopamine-2 receptor agonist;
(5) a dopamine-3 receptor agonist;
(6) a dopamine-4 receptor agonist;
(7) an oxytocin receptor antagonist;
(8) a serotonergic 5HT1B agonist;
(9) a serotonergic 5HT2C agonist;
(10) MT-II;
(11) PT-141;
(12) PT-14;
(13) apomorphine; and
(14) sildenifil;
or a pharmaceutically acceptable salt or ester thereof.

30. The composition according to claim 25 further comprising a pharmaceutically acceptable carrier.

31. A method of treating obesity in a subject comprising administration of:
(a) a therapeutically effective amount of a neurokinin-1 antagonist, or a pharmaceutically acceptable salt or ester thereof; and
(b) a therapeutically effective amount of an anti-obesity agent, or a pharmaceutically acceptable salt or ester thereof; to a subject in need of such treatment,
provided that the anti-obesity agent is not selected from the group consisting of: selective serotonin reuptake inhibitor, fluvoxamine, paroxetine, sertraline, a minorex, amphechloral, amphetamine, benzphetamine, p-chloroamphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, fluoxetine, furfurylmethyl-amphetamine, levamfetamine, levophacetoperane, mazindol, mefenorex, metamfepramone, methamphetamine, norpseudoephedrine, pentorex, phendimetrazine, phenmetrazine, phentermine, phenylpropanolamine, picilorex and sibutramine, and pharmaceutically acceptable salts thereof.

32. The method of claim 31 wherein the anti-obesity agent is selected from the group consisting of:
(1) CB-1 antagonist/inverse agonist;
(2) ghrelin antagonist;
(3) H3 antagonist/inverse agonist;
(4) MCH1R antagonist;
(5) MCH2R agonist/antagonist;
(6) MC3R agonist;
(7) MC4R agonist;
(8) Neuromedin U 1 receptor agonist;
(9) Neuromedin U 2 receptor agonist;
(10) NPY1 antagonist;
(11) NPY2 agonist;
(12) NPY4 agonist;
(13) NPY5 antagonist;
(14) leptin;
(15) leptin agonist/modulator;
(16) leptin derivatives;
(17) opioid antagonist;
(18) orexin antagonist;
(19) BRS3 agonist;
(20) 11 HSD-1 inhibitor;
(21) CCK-A agonist;
(22) CNTF;
(23) CNTF agonist/modulator;
(24) CNTF derivative;
(25) DP-IV inhibitor;
(26) GHS agonist;
(27) UCP-1, 2, and 3 activator;
(28) β3 agonist;
(29) thyroid hormone β agonist;
(30) FAS inhibitor;
(31) DGAT1 inhibitor;
(32) DGAT2 inhibitor;
(33) ACC2 inhibitor;
(34) glucocorticoid antagonist;
(35) acyl-estrogens;
(36) lipase inhibitor;
(37) fatty acid transporter inhibitor;
(38) dicarboxylate transporter inhibitor;
(39) glucose transporter inhibitor;
(40) GLP-1 agonist;
(41) axokine;
(42) metformin;
(43) nalmefene;
(44) phytopharm compound 57;
(45) topiramate; and
(46) zonisamide;
or a pharmaceutically acceptable salt or ester thereof.

33. The method of claim 31 wherein the anti-obesity agent is a melanocortin 4 receptor agonist, or a pharmaceutically acceptable salt or ester thereof.

34. A method of treating an obesity-related disorder in a subject comprising administration of:
(a) a therapeutically effective amount of a neurokinin-1 antagonist, or a pharmaceutically acceptable salt or ester thereof, and
(b) a therapeutically effective amount of an anti-obesity agent selected from the group consisting of:
(1) CB-1 antagonist/inverse agonist;
(2) ghrelin antagonist;
(3) H3 antagonist/inverse agonist;
(4) MCH1R antagonist;
(5) MCH2R agonist/antagonist;
(6) MC3R agonist;
(7) MC4R agonist;
(8) Neuromedin U 1 receptor agonist;
(9) Neuromedin U 2 receptor agonist;
(10) NPY1 antagonist;
(11) NPY2 agonist;
(12) NPY4 agonist;
(13) NPY5 antagonist;
(14) leptin;
(15) leptin agonist/modulator;
(16) leptin derivatives;
(17) opioid antagonist;
(18) orexin antagonist;
(19) BRS3 agonist;
(20) 11β HSD-1 inhibitor;
(21) CCK-A agonist;
(22) CNTF;
(23) CNTF agonist/modulator;
(24) CNTF derivative;
(25) DP-IV inhibitor;
(26) GHS agonist;
(27) UCP-1, 2, and 3 activator;
(28) β3 agonist;
(29) thyroid hormone β agonist;
(30) FAS inhibitor;
(31) DGAT1 inhibitor;
(32) DGAT2 inhibitor;
(33) ACC2 inhibitor;
(34) glucocorticoid antagonist;
(35) acyl-estrogens;
(36) lipase inhibitor;
(37) fatty acid transporter inhibitor;
(38) dicarboxylate transporter inhibitor;
(39) glucose transporter inhibitor;
(40) GLP-1 agonist;
(41) axokine;
(42) metformin;
(43) nalmefene;
(44) phytopharm compound 57;
(45) topiramate; and
(46) zonisamide;
or a pharmaceutically acceptable salt or ester thereof, to a subject in need of such treatment.

35. The method of claim 34 wherein the anti-obesity agent is a melanocortin 4 receptor agonist, or a pharmaceutically acceptable salt or ester thereof.

36. The method of claim 34 wherein the obesity-related disorder selected from: overeating; bulimia; hypertension; diabetes, elevated plasma insulin concentrations; insulin resistance; dyslipidemia; hyperlipidemia; endometrial, breast, prostate and colon cancer; osteoarthritis; obstructive sleep apnea; cholelithiasis; gallstones; coronary heart disease; abnormal heart rhythms; heart arrythmias; myocardial infarction; polycystic ovarian disease; craniopharyngioma; the Prader-Willi Syndrome; Frohlich's syndrome; GH-deficient subjects; normal variant short stature; Turner's syndrome; metabolic syndrome; and acute lymphoblastic leukemia.

37. The method of claim 34 wherein the obesity-related disorder is diabetes.

38. The method of claim 34 wherein the obesity-related disorder is metabolic syndrome.

39. The method of claim 34 wherein the subject in need of such treatment is suffering from emesis.

40. The method of claim 39 wherein the emesis is caused by the administration of the anti-obesity agent, or a pharmaceutically acceptable salt or ester thereof.

41. A method of treating sexual dysfunction in a subject comprising administration of:
(a) a therapeutically effective amount of a neurokinin-1 antagonist, and pharmaceutically acceptable salts and esters thereof; and
(b) a therapeutically effective amount of a sexual dysfunction therapeutic agent, and pharmaceutically acceptable salts and esters thereof;
to a subject in need of such treatment.

42. The method of claim 41 wherein the sexual dysfunction therapeutic agent is selected from the group consisting of:
(1) a type V cyclic-GMP-selective phosphodiesterase inhibitor;
(2) an α₁-adrenergic receptor antagonist;
(3) an α₂-adrenergic receptor antagonist;
(4) a dopamine-2 receptor agonist;
(5) a dopamine-3 receptor agonist;
(6) a dopamine-4 receptor agonist;
(7) an oxytocin receptor antagonist;
(8) a serotonergic 5HT1B agonist;
(9) a serotonergic 5HT2C agonist;
(10) MT-II;
(11) PT-141;
(12) PT-14;
(13) apomorphine; and
(14) sildenifil;
and pharmaceutically acceptable salts and esters thereof.

43. The method of claim 41 wherein the sexual dysfunction therapeutic agent is a melanocortin 4 receptor agonist, or a pharmaceutically acceptable salt or ester thereof.

44. The method of claim 41 wherein the sexual dysfunction is male erectile dysfunction.

45. The method of claim 41 wherein the sexual dysfunction is female sexual dysfunction.

46. A kit comprising at least one unit dosage of a prophylactically or therapeutically effective amount of a neurokinin-1 antagonist, or a pharmaceutically acceptable salt or ester thereof, and at least one unit dosage of a prophylactically or therapeutically effective amount of a melanocortin 4 receptor agonist, or a pharmaceutically acceptable salt or ester thereof.
